(19) United States
(12) Patent Application Publication
(10) Pub. No.: US 2006/0252042 A1
(43) Pub. Date: Nov. 9, 2006

(54) Methods of treatment of feeding disorders or disorders of glucose uptake and for modifying metabolism and identifying therapeutic reagents therefor

(75) Inventor: Juan Carlos Molero, Chiswick, New South Wales (AU)

Correspondence Address: QUINE INTELLECTUAL PROPERTY LAW GROUP, P.C., P O BOX 458, ALAMEDA, CA, 94501, US

(73) Assignee: Garvan Institute of Medical Research, Darlinghurst, New South Wales (AU)

(21) Appl. No.: 10/539,105

(22) PCT Filed: Dec. 16, 2003
(86) PCT No.: PCT/AU03/01676

§ 371 Date: Apr. 14, 2006

(30) Foreign Application Priority Data
Dec. 16, 2002 (AU) 2002953393
Nov. 14, 2003 (AU) 2003906285

(51) Int. Cl. C12Q 1/68; G01N 33/567

(52) U.S. Cl. 435/6; 435/7.2

(57) ABSTRACT

The present invention provides several assay formats for determining the effect of compounds on one or more metabolism-associated phenotypes in a cell, tissue or animal. The assay formats are based upon the finding that the proto-oncogene Cbl is involved in modulating basal glucose uptake, metabolic rate, adipogenesis, muscle thermogenesis, mitochondrial structure and function, the ratio of lean muscle mass to body fat and feeding behavior.

### FIELD OF THE INVENTION

The present invention relates generally to the treatment of feeding disorders and disorders of glucose uptake or metabolism, such as, for example, diabetes, obesity, anorexia or bulimia, in humans and other animals. More particularly, this invention provides methods of modifying adipose tissue (e.g., in connection with treating diabetes, obesity, or hypolipidemia) and/or feeding behavior (e.g in connection with treating overeating, bulimia or anorexia). The invention also relates to method for identifying modulators of glucose uptake or metabolism that are useful in the therapeutic methods described herein, e.g. using a non-human animal model.

### BACKGROUND TO THE INVENTION

1. General

This specification contains nucleotide and amino acid sequence information prepared using PatentIn Version 3.1, presented herein after the claims. Each nucleotide sequence is identified in the sequence listing by the numeric indicator <210> followed by the sequence identifier (e.g. <210>1, <210>2, <213> etc). The length and type of sequence (DNA, protein (PRT), etc), and source organism for each nucleotide sequence, are indicated by information provided in the numeric indicator fields <211>, <212> and <213>, respectively. Nucleotide-sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAG-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

The embodiments of the invention described herein with respect to any single embodiment shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally equivalent products, compositions and methods are clearly within the scope of the invention, as described herein.

The present invention is performed without undue experimentation using, unless otherwise indicated, conventional, techniques of molecular, biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactive, Germany);
10. Sakakibara, D., Teichman, J., Lien, E. Land Fenichel, R. L. (1976). Biochem. Biophys. Res. Commun. 73-336-342
11. Merrifield, R. B. (1963). J. Am. Chem. Soc. 85, 2149-2154.
12. Barany, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müler E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M & Bodanszky, A. (19984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).
   2. Description of the Related Art

The c-Cbl protein is a multi-adaptor protein that is involved in ligand-induced down regulation of receptor tyrosine kinases. The interaction between c-Cbl and its many binding partners involves particular c-Cbl protein domains. All Cbl proteins have a high degree of sequence homology between their tyrosine kinase-binding, linker and RING finger domains, and most have extensive proline-rich regions in their carboxy-terminal halves. The tyrosine kinase-binding domain is composed of three interacting domains comprising a four-helix bundle, a Ca²⁺-binding EF hand, and an atypical or variant Src homology region 2 (SH2) domain that is connected to the RING finger by a short linker domain. A ubiquitin-associated (UBA)/LZ domain at the carboxyl terminus of c-Cbl, Cbl-b and D-Cbl has homology to known ubiquitin-associated domains and leucine, zippers and is believed to be involved in Cbl-mediated ubiquitination of active receptors, which is essential for receptor degradation and turnover, thereby leading to cessation of downstream signalling from the receptor (Soubeyran et al., Nature 416, 183-187; 2002).

Diabetes, and conditions related thereto such as obesity, are major health concerns throughout the world, and contribute to morbidity and mortality. Non-insulin dependent diabetes mellitus (NIDDM or type II diabetes), is the major form of diabetes in developed countries, however efficient means of therapeutic intervention are lacking. While a large number of environmental and genetic factors contribute to the risk of NIDDM in the United States, prolonged obesity is by far the largest risk factor.

A variety of factors contribute to insulin resistance in laboratory animals, as well as in humans, including a sedentary lifestyle, a diet high in either fat or carbohydrate. A common feature of these risk factors is that the energy balance shifts in favour of, energy storage usually in the form of lipid. The intracellular accumulation of lipid is toxic to many cells such as pancreatic β-islet cells and this is considered to be an important factor in the progression of NIDDM.

To address these problems, the pharmaceutical industry has focused its efforts on identifying compounds that target a variety of metabolic endpoints, including insulin resistance, food intake and nutrient absorption. However, relatively few drugs have made it through clinical trials and most of these have unpleasant side effects. Insulin-sensitizing compounds that have been identified to target insulin resistance associated with both obesity and NIDDM often lead to increased body weight and so ultimately these may exacerbate some of the problems associated with these disorders. The insulin-sensitising compounds appear to increase adipocyte differentiation.

Before the development of diabetes, many obese patients develop a peripheral resistance to the actions of insulin. The molecular basis of insulin-resistance in obesity has been the subject of intensive study, but nonetheless remains elusive. Insights into components and mechanisms of the link between obesity and insulin resistance have been gained from mouse models of obesity which display obesity-induced insulin resistance. The molecular basis of the various mouse obesity models covers a range of mechanisms; nonetheless these all develop diabetes, either before or after the onset of obesity.

Obesity in humans and rodents is also commonly associated with insulin resistance in fat and muscle cells (LeRoith et al., Diabetes Mellitus: a Fundamental and Clinical Text. (Lippincott-Raven, Philadelphia, 1996); DeFronzo et al., Diabetes Care, 15:318-68 (1992); Rifkin et al., Diabetes Mellitus, (Elsevier, N.Y., 1990)).

Much work has focussed on the insulin-sensitive glucose transporter GLUT4 (Watson and Pessin, Exp. Cell Res. 271: 75-83, 2001; Bogan et al. published U.S. Patent Application No. 20020052012); Insulin binds to the insulin receptor (IR) in the plasma membrane, where it activates tyrosine kinase in a cascade of events involving phosphatidylinositol-3-kinase (PI 3-K)-mediated recruitment of GLUT4 to the cell surface Min et al., Mol. Cell. 3: 751-760, 1999, Olson et al., Mol. Cell. Biol. 17: 2425-2435, 1997; Hausdorff et al., J. Biol. Chem. 270: 12965-12968, 1995; Elmendorf et al., J. Biol. Chem. 273: 13289-13296, 1998; Holman et al., J. Biol. Chem. 265: 18172-18179, 1990; Piper et al., Am. J. Physiol. 260: C570-C580). The activation of PI 3-K However, PI-K3-mediated trafficking of GLUT4 is not sufficient to explain the extent of insulin resistance (Pessin et al., J. Clin. invest. 106: 165-169, 2000). For example, other growth factors and adhesion molecules that can activate PI 3-K and its downstream kinases (ie. AKT, protein kinase Cζ/λ (PKCζ/λ)) have no effect on glucose transport or GLUT4 translocation. Additionally, mutant mice lacking GLUT4 develop only mild hyperinsulinemia (Katz et al., Nature 377:151-155, 1995).

A second PI 3K-independent signalling cascade, initiated by recruitment of c-Cbl (Langdon et al., J. Virol. 63: 5420-5424, 1989; Langdon et al., Proc. Natl Acad. Sci. USA 86: 1168-1172, 1989) to the insulin receptor, has been postulated to be involved in insulin-stimulated glucose transport and uptake in fat and muscle cells. In this pathway, the c-Cbl protein is also recruited to the insulin receptor by interaction with the adaptor protein CAP, through one of three SH3 domains in the carboxyl-terminus of CAP. c-Cbl is then phosphorylated by the receptor, and the CAP-Cbl complex dissociates from the insulin receptor and moves to a caveolin-enriches triton insoluble membrane fraction. Based upon two-hybrid assay data measuring in vitro protein-protein associations Baumann et al., Nature 407: 202-207, 2000, showed that c-Cbl forms a ternary complex with two other proteins, CAP and flotillin. The interaction with flotillin directs the CAP-Cbl complex to the lipid raft sub-domain of the plasma membrane. Baumann et al. Nature 407: 202-207, 2000, also showed that both insulin-stimulated glucose transport and GLUT4 translocation to the cell surface are attenuated by about 50% in 3T3-L1 adipocytes, by expression of a truncated CAP protein lacking SH3 domains (i.e. CAPΔSH3). If this pathway were to operate in that cells and/or muscle cell in vivo, it would be expected that insulin-induced glucose uptake and its subsequent incorporation into both glycogen and lipid would be impaired in situations which disrupt formation or activity of the c-Cbl-CAP-flotillin complex.

An interaction between c-Cbl and APS, an adaptor protein having a PH domain and SH2 domain (Ahmed et al., Biochem. J. 341, 665-668, 1999) is required for c-Cbl to bind to the insulin receptor in the caveolae-small invaginations in the plasma membrane that are a subset of the lipid raft domains. In this pathway, APS interacts with the phosphorylated insulin receptor via its SH2 domain, and subsequently undergoes tyrosine phosphorylation at a specific residue in the C-terminus of the protein. Upon phosphorylation, APS recruits c-Cbl to the receptor through an atypical SH2 domain of c-Cbl (Saltiel and Pessin, TRENDS Cell Biol. 12, 65-71, 2002);

Based upon co-localization studies, Chiang et al., Nature 410: 944-948, 2001 also showed that the time-course for the insulin-stimulated migration of c-Cbl parallels movement of the SH2-containing adaptor protein CrkII, and the guanyl nucleotide exchange factor C3G, into the caveolin-enriches triton insoluble membrane fraction. Chiang et al. also demonstrated that C3G exchanges GTP for GDP on TC10, a Rho-family GTP-binding protein that regulates GLUT4 transport. In this pathway, phosphorylated c-Cbl recruits CrkII to the lipid rafts, along with C3G to facilitate activation of TC10 by C3G (Chiang et al., Nature 410: 944-948, 2001).

No direct role for c-Cbl in modulating glucose uptake or GLUT4 translocation has been demonstrated in muscle and fat cells in vivo. This is because the binding studies by Baumann et al. and Chiang et al. supra were carried out in isolated 3T3L1 adipocytes and do not suggest that equivalent effects occur in vivo, let alone in both muscle and fat cells of animals. It is also unclear whether the effects reported by Baumann et al. for the over expression of CAPΔSH3 protein in isolated adipocytes were a direct consequence of CAP failing to bind c-Cbl or to a secondary effect of expressing the mutant CAPΔSH3 protein. For example, the expressed CAPΔSH3 protein may have modified the ability of endogenous CAP to bind other proteins in the lipid rafts or elsewhere in the cell. Over expression of the CAPΔSH3 protein also decreases insulin-stimulated recruitment of C3G into lipid rafts, and reduces the basal level of activated TC10 in 3T3-L1 adipocytes (Chiang et al., Nature 410: 944-948, 2001). Accordingly, no-clear-direct-role has emerged for c-Cbl in modulating glucose, uptake and incorporation into lipid in both fat cells and muscle cells of animals. Thus, it is not possible at present to conclude that impaired c-Cbl-CAP-flotillin complex formation and activity are sufficient to produce insulin resistance in humans and other animals.

A number of mouse models have been developed having genetic obesity-diabetes syndromes (Herberg, et al., Metabolism 26: 59-99, 1977). The mice typically have hyperglycemia, hyperinsulinemia, and obesity, albeit to different degrees, with different times of onset, and for different reasons. In the yellow obese mouse (A^{y/a}), a dominant mutation causes the ectopic, ubiquitous expression of the agouti protein, resulting in a condition similar to adult-onset obesity and non-insulin-dependent diabetes mellitus (Michaud et al., Proc Natl Acad Sci USA 91: 2562-2566, 1994). Obese (ob/ob) (Zhang et al., Nature 372: 425-432, 1994); diabetes (db/db) (Tartaglia et al., Cell 83: 1263-1271, 1995), adipose (cpe/cpe) (Naggert et al., Nat. Genet. 10: 135-142, 1995) and tubby (tub/tub) (Kleyn et al., Cell 85: 281-290, 1996; Noben-Trauth et al., Nature 380: 534-548, 1996) are mutations in the genes encoding leptin, the leptin receptor, carboxypeptidase E, and a member of a new family of genes encoding tubby-like proteins, respectively. Obese mice exhibit hyperglycemia, glucose intolerance, and elevated plasma insulin, which develops after the onset of obesity. In db/db mice, elevation of plasma insulin occurs at 2 weeks of age, preceding the onset of obesity at 3-4 weeks and elevation of blood glucose levels at 4-8 weeks. Adipose mice have hyperinsulinemia throughout life in association with hypertrophy and hyperplasia of the islets of Langerhans, with transient hyperglycemia. Tubby mice have normal blood glucose, however plasma insulin is elevated prior to obvious signs of obesity, and the islets of Langerhans are enlarged.

Mouse models having impaired glucose uptake are highly desirable. By virtue of examining their phenotype, such models would have utility in determining appropriate targets for the therapy of a wide range of disorders associated with aberrant glucose metabolism and for determining the efficacy or specificity of therapeutics.

### SUMMARY OF THE INVENTION

In work leading up to the present invention, the inventors studied the role of Cbl, particularly c-Cbl, in modulating one or more metabolism-associated phenotypes in a cell, tissue or animal, e.g., in insulin resistance, obesity and diabetes in vivo. Surprisingly, the inventors found that a targeted disruption of a Cbl locus in mice sufficient to prevent functional Cbl protein from being expressed, does not disrupt, has little or no defect on basal or insulin-induced glucose uptake and incorporation of glucose into lipid in adipocytes. In muscle cells, Cbl-deficient mice were found to have elevated basal glucose uptake, consistent with an enhanced metabolic rate. Cbl-deficient mice had significantly smaller adipocytes than wild-type counterparts of the same approximate weight. Consistent with this conclusion, the Cbl-deficient mice also had an elevated temperature relative to normal mice, suggesting that the enhanced ratio of lean muscle mass to body fat in Cbl-deficient mice is a consequence of enhanced metabolic rate. It was also surprising that the feeding behavior of the animals was markedly modified, as Cbl-deficient mice exhibited a markedly enhanced appetite as determined by the amount of food consumed per day (dietary intake). These data also indicate that Cbl, such as, for example, c-Cbl, is directly involved in modulating the feeding behavior of animals and in regulating fat deposition in adipocytes in mammals. The Cbl protein and Cbl-deficient mouse model are therefore useful for identifying compounds that specifically modulate a Cbl-mediated characteristic such as, for example, feeding behavior (e.g. in the treatment of anorexia or bulimia), fat deposition, metabolic rate, the ratio of lean muscle mass to body fat, or glucose uptake (e.g. in the treatment of obesity or type II diabetes). These modulators are identified by screening animals for their effect on the phenotype of a suitable animal model, e.g., the Cbl^{-/-} mouse model or a derivative thereof expressing human c-Cbl protein, or alternatively, by directly screening the Cbl protein for altered function, including but not limited to any specific known functions of Cbl such as, for example, binding activity, ubiquitin ligase activity, or by screening for the effect of the compound on the extent of tyrosine phosphorylation of Cbl.

Methods for determining the effect of a compound on a metabolism-associated phenotype of the mouse model will be apparent from the morphology and muscle thermogenesis phenotypes described herein in Example 1-4. By “metabolism-associated phenotype” is meant a phenotype associated with Cbl activity and/or expression per se such as, for example, Cbl-mediate ubiquitination of the insulin receptor, tyrosine phosphorylation of Cbl, Cbl protein level, and/or Cbl-mediated fat and/or glucose metabolism, such as, for example, a phenotype selected from the group consisting of fat mass, glucose transport, muscle thermogenesis, muscle temperature, mitochondrial structure, mitochondrial function, and mitochondrial respiration rate.

Accordingly, one aspect of the present invention provides a method of identifying a compound that suppresses or reduces feeding behavior, such as, for example, in the treatment of obesity, said method comprising: (a) administering a compound to a genetically modified non-human animal comprising a genetic modification within an allele of its Cbl locus wherein said genetic modification reduces or prevents expression of a functional endogenous Cbl in said animal; and (b) determining the feeding behavior of the animal, wherein reduced appetite or dietary intake of the animal compared to the appetite or dietary intake of a Cbl-deficient animal to which the compound has not been administered indicates that the compound suppresses or reduces feeding behavior.

In an alternative embodiment, the present invention provides a method for identifying a compound that suppresses or reduces feeding behavior, such as, for example, in the treatment of obesity, said method comprising determining the ubiquitin ligase activity of a Cbl protein in the presence and absence of the compound wherein enhanced ubiquitin ligase activity of the Cbl protein in the presence of the compound indicates that the compound suppresses or reduces feeding behavior. In an alternative embodiment, the present invention provides a method for identifying a compound that suppresses or reduces feeding behavior comprising determining the level of tyrosine phosphorylation of a Cbl protein in the presence and absence of the compound wherein enhanced phosphorylation of tyrosine residues in the Cbl protein in the presence of the compound indicates that the compound suppresses or reduces feeding behavior.

Another embodiment of the invention provides a method of identifying a compound that enhances feeding behavior, such as, for example, in the treatment of anorexia or bulimia, said method comprising: (a) administering a compound that suppresses appetite or dietary intake to a genetically modified non-human animal comprising a genetic modification within an allele of its Cbl locus wherein said genetic modification reduces or prevents expression of a functional endogenous Cbl in said animal and determining the feeding behavior of the animal; (b) administering a compound to the animal and determining the feeding behavior of the animal, wherein enhanced appetite or dietary intake at (b) compared to (a) indicates that the compound enhances feeding behavior.

In an alternative embodiment, the invention provides a method of identifying a compound that enhances feeding comprising determining the ubiquitin ligase activity of a Cbl protein in the presence and absence of the compound wherein reduced ubiquitin ligase activity of the Cbl protein in the presence of the compound indicates that the compound enhances feeding behavior.

In an alternative embodiment, the present invention provides a method for identifying a compound that enhances feeding behavior comprising determining the level of tyrosine phosphorylation of a Cbl protein in the presence and absence of the compound wherein reduced phosphorylation of tyrosine residues in the Cbl protein in the presence of the compound indicates that the compound enhances feeding behavior.

In an alternative embodiment, the invention provides a method of identifying a compound that modulates feeding behavior, such as, for example, in the treatment of anorexia or bulimia, said method comprising: (a) administering a compound to a non-human animal expressing a functional Cbl protein and determining the feeding behavior of the animal; (b) determining the feeding behavior of a genetically modified non-human animal comprising a genetic modification within an allele of its Cbl locus wherein said genetic modification reduces or prevents expression of a functional endogenous Cbl in said animal; and (c) comparing the feeding behavior of the animals at (a) and (b) wherein a comparable feeding behavior between (a) and (b) indicates that the compound modulates feeding behavior.

In the animal-based assays described herein, it is to be understood that the term “endogenous Cbl” refers to the Cbl encoded by the native genome of the animal, generally other than human Cbl.

In a particularly preferred embodiment of the present invention, an animal-based assay is employed utilizing a genetically modified animal that lacks expression of a functional endogenous Cbl and comprises a gene encoding human Cbl protein. Such animal models are particularly preferred for identifying a modulatory compound and/or for validating the efficacy of any compound or modulator identified using an animal-based assay (e.g., the Cbl^{-/-} mouse) or in vitro assay system. In particular, the use of an animal that ectopically expresses human Cbl protein or is capable of doing so permits the skilled artisan to determine that the compound or modulator targets the expression of the human Cbl gene or activity of the human Cbl protein, or is selective for human Cbl expression and/or activity.

In an alternative embodiment, the invention provides a method of identifying a compound that modulates feeding comprising determining the ubiquitin ligase activity of a Cbl protein in the presence and absence of the compound wherein modified ubiquitin ligase activity of the Cbl protein in the presence of the compound indicates that the compound enhances feeding behavior.

In an alternative embodiment, the present invention provides a method for identifying a compound that modulates feeding behavior comprising determining the level of tyrosine phosphorylation of a Cbl protein in the presence and absence of the compound wherein modified phosphorylation of tyrosine residues in the Cbl protein in the presence of the compound indicates that the compound modulates feeding behavior.

A further aspect of the present invention provides a method of identifying a compound that enhances fat deposition or reduces lean muscle mass or enhances the ratio of body fat to muscle or reduces metabolic rate such as, for example, in the treatment of hypolipidemia (e.g. as observed in subjects suffering from abetalipoproteinemia, malnutrition or hematologic malignancies, such as acute myelocytic leukemia or chronic myeplocytic leukemia), said method comprising: (a) administering a compound to a genetically modified non-human animal comprising a genetic modification within an allele of its Cbl locus wherein said genetic modification reduces or prevents expression of a functional endogenous Cbl in said animal; and (b) determining the fat content of the animal, wherein enhanced fat content of the animal compared to the fat content of a Cbl-deficient animal to which the compound has not been administered indicates that the compound enhances fat deposition or reduces lean muscle mass or enhances the ratio of body fat to muscle or reduces metabolic rate.

In an alternative embodiment, the present invention provides a method of identifying a compound that enhances fat deposition or reduces lean muscle mass or enhances the ratio of body fat to muscle or reduces metabolic rate comprising determining the ubiquitin ligase activity of a Cbl protein in the presence and absence of the compound wherein enhanced ubiquitin ligase activity of the Cbl protein in the presence of the compound indicates that the compound enhances fat deposition or reduces lean muscle mass or enhances the ratio of body fat to muscle or reduces metabolic rate.

In an alternative embodiment, the present invention provides a method for identifying a compound that enhances fat deposition or reduces lean muscle mass or enhances the ratio of body fat to muscle or reduces metabolic rate comprising determining the level of tyrosine phosphorylation of a Cbl protein in the presence and absence of the compound wherein enhanced phosphorylation of tyrosine residues in the Cbl protein in the presence of the compound indicates that the compound enhances fat deposition or reduces lean muscle mass or enhances the ratio of body fat to muscle or reduces metabolic rate.

In an alternative embodiment, the invention provides a method of identifying a compound that reduces fat deposition or enhances lean muscle mass or reduces the ratio of body fat to muscle or enhances metabolic rate, such as, for example, in the treatment of obesity or neurodegenerative disorders or for cosmetic purposes such as bodybuilding or weight loss, said method comprising: (a) administering a compound that enhances fat deposition or glucose uptake to a genetically modified non-human animal comprising a genetic modification within an allele of its Cbl locus wherein said genetic modification reduces or prevents expression of a functional endogenous Cbl in said animal and determining the fat content of the animal; (b) administering a compound to the animal and determining the fat content of the animal, wherein a similar or reduced fat content at (b) compared to (a) indicates that the compound reduces fat deposition or enhances lean muscle mass or reduces the ratio of body fat to muscle or enhances metabolic rate.

In an alternative embodiment, the invention provides a method of identifying a compound that reduces fat deposition or enhances lean muscle mass or reduces the ratio of body fat to muscle or enhances metabolic rate comprising: (a) administering a compound to a non-human animal expressing a functional Cbl protein and determining the fat content of the animal; (b) determining the fat content of a genetically modified on-human animal comprising a genetic modification within an allele of its Cbl locus herein said genetic modification reduces or prevents expression of a functional endogenous Cbl in said animal; and (c) comparing the fat contents of the animals at (a) and (b) wherein a comparable fat content between (a) and (b) indicates that the compound reduces fat deposition or enhances lean muscle mass or reduces the ratio of body fat to muscle or enhances metabolic rate.

In an alternative embodiment, the present invention provides a method of identifying a compound that reduces fat deposition or enhances lean muscle mass or reduces the ratio of body fat to muscle or enhances metabolic rate comprising determining the ubiquitin ligase activity of a Cbl protein in the presence and absence of the compound wherein reduced ubiquitin ligase activity of the Cbl protein in the presence of the compound indicates that the compound reduces fat deposition or enhances lean muscle mass or reduces the ratio of body fat to muscle or enhances metabolic rate.

In an alternative embodiment, the present invention provides a method for identifying a compound that reduces fat deposition or enhances lean muscle mass or reduces the ratio of body fat to muscle or enhances metabolic rate comprising determining the level of tyrosine phosphorylation of a Cbl protein in the presence and absence of the compound wherein reduced phosphorylation of tyrosine residues in the Cbl protein in the presence of the compound indicates that the compound reduces fat deposition or enhances lean muscle mass or reduces the ratio of body fat to muscle or enhances metabolic rate.

A further aspect of the present invention provides a method of identifying a compound that enhances glucose uptake such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a genetically modified non-human animal comprising a genetic modification within an allele of its Cbl locus wherein said genetic modification reduces or prevents expression of a functional endogenous Cbl in said animal; and (b) determining the glucose uptake into liver, fat or muscle cells of the animal, wherein enhanced uptake compared to the glucose uptake into liver, fat or muscle cells of a Cbl-deficient animal to which the compound has not been administered indicates that the compound enhances glucose uptake.

In an alternative embodiment, the present invention provides a method of identifying a compound that enhances glucose uptake comprising determining the ubiquitin ligase activity of a Cbl protein in the presence and absence of the compound wherein reduced ubiquitin ligase activity of the Cbl protein in the presence of the compound indicates that the compound reduces fat deposition or enhances glucose uptake.

In an alternative embodiment, the present invention provides a method for identifying a compound that enhances glucose uptake comprising determining the level of tyrosine phosphorylation of a Cbl protein in the presence and absence of the compound wherein reduced phosphorylation of tyrosine residues in the Cbl protein in the presence of the compound indicates that the compound enhances glucose uptake.

In an alternative embodiment, the invention provides a method of identifying a compound that reduces glucose uptake into liver, fat or muscle cells such as, for example, in the treatment of obesity, said method comprising: (a) administering a compound that enhances glucose uptake to a genetically modified non-human animal comprising a genetic modification within an allele of its Cbl locus wherein said genetic modification reduces or prevents expression of a functional endogenous Cbl in said animal and determining the glucose uptake into liver, fat or muscle cells; (b) administering a compound to the animal and determining the glucose uptake into liver, fat or muscle cells of the animal, wherein a similar or reduced uptake at (b) compared to (a) indicates that the compound reduces glucose uptake into liver, fat or muscle cells.

In an alternative embodiment, the invention provides a method of identifying a compound that reduces glucose uptake into liver, fat or muscle cells comprising: (a) administering a compound to a non-human animal expressing a functional Cbl protein and determining the glucose uptake into liver, fat or muscle cells of the animal; (b) determining the glucose uptake into liver, fat or muscle cells of a genetically modified non-human animal comprising a genetic modification within an allele of its Cbl locus wherein said genetic, modification reduces or prevents expression of a functional endogenous Cbl in said animal; and (c) comparing the glucose uptake into liver, fat or muscle cells of the animals at (a) and (b) wherein a comparable uptake between (a) and (b) indicates that the compound reduces glucose uptake into liver, fat or muscle cells.

In an alternative embodiment, the present invention provides a method of identifying a compound that reduces glucose uptake comprising determining the ubiquitin ligase activity of a Cbl protein in the presence and absence of the compound wherein enhanced ubiquitin ligase activity of the Cbl protein in the presence of the compound indicates that the compound reduces glucose uptake.

In an alternative embodiment, the present invention provides a method for identifying a compound that reduces glucose uptake comprising determining the level of tyrosine phosphorylation of a Cbl protein in the presence and absence of the compound wherein enhanced phosphorylation of tyrosine residues in the Cbl protein in the presence of the compound indicates that the compound reduces glucose uptake.

In a particularly preferred embodiment of the screening assay, the present invention provides a method of identifying a compound that is capable of modulating feeding behavior, fat deposition, metabolic rate, or the ratio of lean muscle mass to body fat in a subject, said method comprising performing an assay to measure a metabolism-associated phenotype that has been determined for a genetically modified non-human animal that comprises a genetic modification within an allele of its Cbl locus wherein said genetic modification reduces or prevents expression of a functional endogenous Cbl in said animal, and wherein said assay is conducted in the presence and absence of a compound to be tested, and determining the effect of the compound on the phenotype wherein a modified phenotype indicates that the compound is capable of modulating feeding behavior, fat deposition, metabolic rate, or the ratio of lean muscle mass to body fat in a subject.

In one embodiment, the assay to determine a metabolism-associated phenotype measures. Cbl-mediated ubiquitination of the insulin receptor in the presence and absence of the compound. Preferably, the assay comprises:
(a) providing a cell that is capable of effecting the c-Cbl-mediated ubiquitination of the insulin receptor;
(b) incubating the cell in the presence and absence of a compound to be tested; and
(c) determining c-Cbl-mediated ubiquitination of the insulin receptor in the presence and absence of the compound wherein a modified level of c-Cbl-mediated ubiquitination of the insulin receptor indicates that the compound is capable of modulating feeding behavior, fat deposition, metabolic rate, or the ratio of lean muscle mass to body fat in a subject.

Preferably, the compound reduces or inhibits Cbl-mediated ubiquitination of the insulin receptor thereby indicating that said compound is capable of enhancing feeding behavior in a subject, reducing fat deposition in a subject, enhancing metabolic rate in a subject, or enhancing the ratio of lean muscle mass to body fat in a subject.

Alternatively, the compound enhances or agonizes Cbl-mediated ubiquitination of the insulin receptor thereby indicating that said compound is capable of reducing feeding behavior and/or enhancing fat deposition in a subject and/or reducing metabolic rate in a subject and/or reducing the ratio of lean muscle mass to body fat in a subject.

In a particularly preferred embodiment, the assay comprises an immunoassay wherein the level of c-Cbl-mediated ubiquitination of the insulin receptor is determined by contacting the insulin receptor with an antibody that binds to ubiquitin under conditions sufficient for an antigen-antibody complex to form and detecting the antibody bound to the receptor. Preferably, the assay further comprises contacting the insulin receptor with an antibody that binds to the insulin receptor under conditions sufficient for an antigen-antibody complex to form.

In an alternative embodiment, the method comprises performing an immunoassay by a process comprising:
(a) providing a cell that is capable of effecting the c-Cbl-mediated ubiquitination of the insulin receptor;
(b) incubating the cell in the presence and absence of a compound to be tested;
(c) contacting an extract of the cell comprising the insulin receptor with an antibody that binds to the insulin receptor under conditions sufficient for an antigen-antibody complex to form thereby capturing the insulin receptor;
(d) contacting the captured insulin receptor with an antibody that binds to ubiquitin under conditions sufficient for an antigen-antibody complex to form; and
(e) detecting the antibody bound at (d).
   In accordance with this embodiment, the antibody bound at (d) is generally detected by contacting the antibody with a tertiary antibody that is capable of producing a detectable signal.

In a further embodiment of the present invention, the assay to determine a metabolism-associated phenotype measures phosphorylation of a tyrosine residue on Cbl protein in the presence and absence of the compound.

In a further embodiment of the present invention, the assay to determine a metabolism-associated phenotype measures the amount of Cbl protein in the cell in the presence and absence of the compound. Preferred assay formats in accordance with this embodiment comprise:
(a) providing a cell that is capable of expressing c-Cbl protein;
(b) incubating the cell in the presence and absence of a compound to be tested; and
(c) determining amount of c-Cbl protein in the cell in the presence and absence of the compound wherein a modified level of c-Cbl protein indicates that the compound is capable of modulating feeding behavior, fat deposition, metabolic rate, or the ratio of lean muscle mass to body fat in a subject.

Preferably, a compound that reduces or inhibits Cbl expression as determined by a reduced amount of Cbl protein in the cell is capable of enhancing feeding behavior, reducing fat deposition, enhancing metabolic rate, or enhancing the ratio of lean muscle mass to body fat in a subject.

Alternatively, a compound that enhances or agonizes Cbl expression as determined by an increased amount of Cbl protein in the cell is capable of reducing feeding behavior and/or enhancing fat deposition in a subject and/or reducing metabolic rate in a subject and/or reducing the ratio of lean muscle mass to body fat in a subject.

In a particularly preferred embodiment, the amount of Cbl protein in a cell is determined by performing an immunoassay wherein the amount of c-Cbl is determined by contacting the Cbl protein with an antibody that binds to Cbl under conditions sufficient for an antigen-antibody complex to form and detecting the antibody bound to the Cbl protein. Preferably, the antibody bound to the Cbl protein is detected by contacting the antibody with a secondary antibody that is capable of producing a detectable signal.

In an alternative embodiment, an immunoassay is performed wherein the amount of c-Cbl is determined by contacting the Cbl protein with a primary and secondary antibody that each bind to Cbl under conditions sufficient for antigen-antibody complexes to form and detecting an antibody bound to the Cbl protein. The antibody bound to the Cbl protein is generally detected by contacting the antibody with a secondary antibody that is capable of producing a detectable signal. The primary and secondary antibody will generally bind to different epitopes on the Cbl protein.

In a further embodiment, the amount of Cbl protein in a cell is detected by an immunoassay comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein; and
(d) detecting the antibody bound at (e).

In a further embodiment, the amount of Cbl protein in a cell is detected by an immunoassay comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein;
(d) contacting the captured Cbl protein with an antibody that binds to Cbl protein under conditions sufficient for an antigen-antibody complex to form, wherein said antibody binds to a different epitope on Cbl to the antibody at (c); and
(e) detecting the antibody bound at (d).

As with these and other preferred immunoassays, the antibody is generally detected by contacting the antibody with an antibody that is capable of producing a detectable signal.

In a further embodiment of the present invention, the assay to determine a metabolism-associated phenotype measures Cbl-mediated fat and/or glucose metabolism in the cell in the presence and absence of the compound. Preferably, the assay to determine a metabolism-associated phenotype measures a phenotype in the presence and absence of the compound selected from the group consisting of fat mass, glucose transport, muscle thermogenesis, mitochondrial structure, mitochondrial function, and mitochondrial respiration rate. Preferably, these parameters are measured in a cell, tissue or animal in the presence and absence of the compound.

As exemplified herein, muscle thermogenesis e.g., in the presence and absence of a test compound, is determined by a process comprising determining the proton leak kinetics of a cell. Accordingly, one particularly preferred embodiment of the measurement of muscle thermogenesis in a cell comprises:
(a) providing a cell of myoblast lineage capable of expressing c-Cbl protein;
(b) incubating the cell in the presence and absence of a compound to be tested; and
(c) determining the respiration rate and/or membrane potential of the cell in the presence and absence of the compound wherein a modified respiration rate and/or membrane potential indicates that the compound is capable of modulating feeding behavior, fat deposition, metabolic rate, or the ratio of lean muscle mass to body fat in a subject.

In accordance with this embodiment, those compounds that enhance respiration rate and/or membrane potential of a cell are capable of enhancing feeding behavior, reducing fat deposition, enhancing metabolic rate, or enhancing the ratio of lean muscle mass to body fat in a subject.

In contrast, compounds that reduce or inhibit respiration rate and/or membrane potential of the cell are capable of reducing feeding behavior and/or enhancing fat deposition in a subject and/or reducing metabolic rate in a subject and/or reducing the ratio of lean muscle mass to body fat in a subject.

In a particularly preferred embodiment, animal models are used to test or validate a putative modulatory compound. In one embodiment, an assay that determines the effect of a compound on a metabolism-associated phenotype comprises:
(a) providing a compound to be tested to an animal subject that expresses a functional Cbl protein; and
(b) determining a metabolism-associated phenotype in a cell or tissue of the animal wherein a modified phenotype in the presence of the compound indicates that the compound is capable of modulating feeding behavior, fat deposition, metabolic rate, or the ratio of lean muscle mass to body fat, in a subject.

Preferably, the metabolism-associated phenotype measured as the assay read-out is selected from the group consisting of fat mass, glucose transport, muscle thermogenesis, mitochondrial structure, mitochondrial function, and mitochondrial respiration rate. As with other embodiments described herein, muscle thermogenesis in the presence of the compound is preferably determined by a process comprising determining the proton leak kinetics of a cell or tissue of the animal. More preferably, muscle thermogenesis is determined by measuring the respiration rate and/or membrane potential of a cell from the animal wherein a modified respiration rate and/or membrane potential indicates that the compound is capable of modulating feeding behavior, fat deposition, metabolic rate, or the ratio of lean muscle mass to body fat in a subject.

In accordance with this embodiment, a compound that enhances respiration rate and/or membrane potential of the cell is capable of enhancing feeding behavior, reducing fat deposition, enhancing metabolic rate, or enhancing the ratio of lean muscle mass to body fat in a subject.

In contrast, a compound that reduces or inhibits respiration rate and/or membrane potential of the cell thereby indicating that said compound is capable of reducing feeding behavior and/or enhancing fat deposition in a subject and/or reducing metabolic rate in a subject and/or reducing the ratio of lean muscle mass to body fat in a subject.

It is particularly preferred that the animal subject used in the screens provided by the present invention is a non-human animal subject, preferably a mammal, such as, for example, a rodent (e.g., a rabbit, rat, guinea pig or mouse), dog, pig, bovine, sheep, horse and goat etc., and more preferably a rabbit, rat or mouse.

In one embodiment, the non-human animal subject expresses an endogenous native Cbl protein (i.e. it is a wild-type animal with respect to Cbl expression). The present invention clearly provides for the use of a Cbl-knock-in animal that expresses an introduced human Cbl protein.

Preferably, a test compound is, administered to muscle tissue of the animal subject. In the case of nucleic acids, it is particularly preferred that these are provided by injection. Preferably, the nucleic acids are contained within a virus vector, in particular an adenovirus. As will be known to those skilled in the art, such adenoviruses tend to remain localized to the injection site. Accordingly, the metabolism-associated phenotype will generally be determined in muscle tissue of the animal subject to which the virus vector is administered.

A further aspect of the present invention provides methods for determining a modulator of the activity, formation or stability of a protein complex selected from the group consisting of: (i) a Cbl-APS complex; (ii) a Cbl-CAP complex; (iii) a Cbl-CAP-flotillin complex; (iv) a Cbl-C3G complex; (v) a Cbl-CrkII complex; and (vi) a Cbl-C3G-CrkII complex.

The present invention clearly encompasses the use of any in silico analytical method and/or industrial process for carrying the screening methods described herein into a pilot scale production or industrial scale production of an inhibitory compound identified in such screens. This invention also provides for the provision of information for any such production. Accordingly, a further aspect of the present invention provides a process for identifying or determining a compound or modulator supra, said method comprising:
(i) performing a method as described herein to thereby identify or determine a compound or modulator;
(ii) optionally, determining the structure of the compound or modulator; and
(iii) providing the compound or modulator or the name or structure of the compound or modulator such as, for example, in a paper form, machine-readable form, or computer-readable form.
   Naturally, for compounds that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the compound or modulator” shall be taken to include any chemical or recombinant synthetic means for producing said compound or modulator or alternatively, the provision or a compound or modulator that has been previously synthesized by any person or means.

In a preferred embodiment, the compound or modulator or the name or structure of the compound or modulator is provided with an indication as to its use e.g., as determined by a screen described herein.

A further aspect of the present invention provides a process for producing a compound or modulator supra, said method comprising:

a process for identifying or determining a compound or modulator supra, said method comprising:

(i) performing a method as described herein to thereby identify or determine a compound or modulator;
(ii) optionally, determining the structure of the compound or modulator;
(iii) optionally, providing the name or structure of the compound or modulator such as, for example, in a paper form, machine-readable form, or computer-readable form; and
(iv) producing or synthesizing the compound or modulator.

In a preferred embodiment, the synthesized compound or modulator or the name or structure of the compound or modulator is provided with an indication as to its use e.g., as determined by a screen described herein.

In a further aspect, the invention provides methods relating to the treatment of animal or human subjects wherein a compound that modulates (i.e. enhances or reduces or prevents) Cbl expression or activity is administered to the animal. Such methods apply mutatis mutandis to the treatment of a wide range of conditions associated with Cbl function, such as, for example, hyperglycemia, hyperinsulinemia, obesity, adult-onset obesity, non-insulin-dependent diabetes mellitus, type II diabetes, glucose intolerance, hypertrophy or hyperplasia of the islets of Langerhans, or for cosmetic purposes, such as, for example, bodybuilding or weight management (i.e. weight loss or weight gain). It is to be understood that such applications of the invention also relate to the productivity of stock and farm animals (e.g. dairy and beef cattle, pigs, horses, sheep, etc) such as, for example, for modulating the amount of fat they deposit or their ratio of fat to muscle mass. In accordance with this aspect of the invention, an amount of a Cbl agonist or antagonist is administered to the animal or human subject effective to modulate the expression or activity of Cbl in the subject.

In another embodiment, the invention also provides a method of treating a feeding disorder characterized by reduced dietary intake or suppressed appetite in a subject said method comprising administering to the subject an amount of a Cbl antagonist effective to enhance the appetite or dietary intake of the subject. The method of the invention is particularly suited to the treatment of anorexia or bulimia.

In a related embodiment, the invention also provides a method of treating a feeding disorder characterized by reduced dietary intake or suppressed appetite in a subject said method comprising administering to the subject an amount of a compound that reduces expression of functional Cbl effective to enhance the appetite or dietary intake of the subject.

### BRIEF DESCRIPTION OF THE DRAWINGS

FIG. 1 is a graphical representation showing the body weight in grams (ordinate) of male (circles) and female (triangles) wild type mice (open symbols), and Cbl-deficient male (circles) and female (triangles) mice that are homozygous for a mutation in both alleles of the Cbl locus (filled symbols). Body weights of mice were determined every week from weaning to 16 weeks of age (x-axis). Data indicate that Cbl-deficient male mice have significantly higher body weight than their wild-type counterparts, throughout the time period tested. The weights of Cbl-deficient female mice are comparable to the weights of wild-type females.

FIG. 2 is a graphical representation showing the dietary food intake in grams per day (ordinate) of male (circles) and female (triangles) wild type mice (open symbols), and Cbl-deficient male (circles) and female (triangles) mice that are homozygous for a mutation in both alleles of the Cbl locus (filled symbols). Food intake was determined every week from 5 weeks of age until 16 weeks of age (x-axis). Data indicate that Cbl-deficient male and female mice have significantly enhanced dietary intake (i.e. enhanced appetite) than their wild-type counterparts, throughout the time period tested. The dietary intake of Cbl-deficient female mice is also significantly higher than the dietary intake of wild-type males.

FIG. 3 is a graphical representation showing the dietary food intake relative to body weight (ordinate) of male (circles) and female (triangles) wild-type mice (open symbols), and Cbl-deficient male (circles) and female (triangles) mice that are homozygous for a mutation in both alleles of the Cbl locus (filled symbols). Food intake relative to body weight was determined every week from 5 weeks of age until 16 weeks of age (x-axis). Data indicate that Cbl-deficient male and female mice have significantly enhanced specific, dietary intake (i.e. enhanced appetite as determined by food intake relative to body weight) than their wild-type counterparts, throughout the time period tested.

FIG. 4 is a tabular representation showing the tissue weights (average+/−SEM) of wild type mice and Cbl-deficient mice that are homozygous for a mutation in both alleles of the Cbl locus (filled symbols). WAT, white adipose tissue; BAT, brown adipose tissue; QUAD, quadriceps muscle. Data indicate reduced adipose tissue weight for Cbl-deficient animals relative to wild type animals.

FIG. 5 is a photographic representation showing adipocyte size in wild type mice (left) and Cbl-deficient mice that are homozygous for a mutation in both alleles of the Cbl locus (right).

FIG. 6 is a tabular representation showing adipocyte diameter (μm) adipocyte volume (pl/cell) and lipid content (ng/cell) of male (Top panel) and female (Lower panel) wild type mice (WT), compared to male (Top panel) and female (Lower panel) Cbl-deficient mice that are homozygous for a mutation in both alleles of the Cbl locus (KO). Data indicate the mean values+/−SEM. Data indicate that for both males and females, there is a significant reduction in adipocyte size and volume, and reduced lipid content of adipocytes in Cbl-deficient mice relative to wild-type mice.

FIG. 7A is a graphical representation showing glucose transport in soleus muscles of c-CBL^{-/-} mice. Soleus muscles were removed from c-CBL−/− mice and wild-type c-CBL+/+ mice and incubated in the presence of labeled 2-deoxyglucose and no insulin (basal), 300 μU/ml insulin (submax) and, 10 μU/ml insulin (supramax). The muscle was then liquefied and the amount of labeled 2-deoxyglucose taken up by the muscle determined using a liquid scintillation counter. This amount+/−SEM was then graphically represented. *, p<0.05; *, p<0.01.

FIG. 7B is a graphical representation showing glucose transport in extensor digitorum longus muscles of c-CBL^{-/-} mice. Extensor digitorum longus muscles were removed from c-CBL−/− mice and wild-type c-CBL+/+ mice and incubated in the presence of labeled 2-deoxyglucose and no insulin (basal), 300 μU/ml insulin (submax) and 1000 μU/ml insulin (supramax). The muscle was then liquefied and the amount of labeled 2-deoxyglucose taken up by the muscle determined using a liquid scintillation counter. This amount+/−SEM was then graphically represented. *, p<0.05.

FIG. 8 is a graphical representation showing glucose transport in fat explants taken from c-CBL^{-/-} mice. Epididymal fat pads removed from c-CBL−/− mice and wild-type c-CBL+/+ mice and minced. Samples were then incubated in the presence of labeled 2-deoxyglucose and no insulin (0), 0.05 nM insulin (0.05) and 1 nM insulin (1). The amount of labeled 2-deoxyglucose taken up by the fat explant was determined using a liquid scintillation counter. This amount+/− SEM was then graphically represented.

### DETAILED DESCRIPTION OF THE PREFERRED EMBODIMENTS

One aspect of the present invention provides a method of identifying a compound that suppresses or reduces feeding behavior, such as, for example, in the treatment of obesity, said method comprising: (a) administering a compound to a genetically modified non-human animal comprising a genetic modification within an allele of its Cbl locus wherein said genetic modification reduces or prevents expression of a functional endogenous Cbl in said animal; and (b) determining the feeding behavior of the animal, wherein reduced appetite or dietary intake of the animal compared to the appetite or dietary intake of a Cbl-deficient animal to which the compound has not been administered indicates that the compound suppresses or reduces feeding behavior.

As used herein, the term: “Cbl” shall be taken to mean any peptide, polypeptide, or protein having at least about 80% amino acid sequence identity to the amino acid sequence of a human or mouse c-Cbl polypeptide set forth in SEQ ID NO: 2 or 3. The term “Cbl” shall also be taken to include a peptide, polypeptide or protein having the known biological activity of Cbl, or the known binding specificity of Cbl including c-Cbl. For the purposes of nomenclature, the amino acid sequences of the murine and human Cbl polypeptides are exemplified herein, as SEQ ID Nos: 2 and 3, respectively. Preferably, the percentage identity to SEQ ID NO: 2 or 3 is at least about 85%, more preferably at least about 90%, even more preferably at least about 95% and still more preferably at least about 99%.

A “Cbl” protein generally comprises about 906 amino acid residues in length. The full-length protein generally comprises a tyrosine kinase binding domain that binds the protein to phosphotyrosine residues, thereby coupling Cbl to growth factor receptor signalling. For example, the c-Cbl is phosphorylated on 3-4 tyrosine residues in response to growth factors, the phosphorylated residues representing binding domains for SH2 domain containing proteins such as PI 3′ kinase and Crk II. The tyrosine kinase binding domain generally will comprise a four helix bundle, an EF hand domain and an SH2 domain. Downstream of the tyrosine kinase binding domain is generally a C3HC4 RING finger domain having high sequence similarity to that found in ubiquitin ligase proteins. At the C terminal portion of the protein there is generally a proline rich region (PRR) which can bind SH3 domain-containing proteins (e.g. CAP). At the extreme C terminus of Cbl there is generally located a leucine zipper domain (b-Zip) and a ubiquitin association domain that regulates homodimerization of Cbl.

The various embodiments of the present invention directed to the identification of compounds that modulate feeding behavior (e.g. in the treatment of obesity, anorexia or bulimia), fat depositions metabolic rate, the ratio of lean muscle mass to body fat, or glucose uptake (e.g. in the treatment of obesity or type II diabetes), can also be carried out by determining a physical property of the Cbl protein, or alternatively, a catalytic activity of the Cbl protein, that is modified during glucose uptake into fat cells or muscle cells.

The ability of a compound to enhance feeding behavior, reduce fat deposition, enhance metabolic rate, or enhance the ratio of lean muscle mass to body fat can thus be readily assayed by determining whether or not the compound reduces a Cbl activity that is disrupted in the Cbl-deficient mouse model. For example, reduced ubiquitin ligase activity of Cbl protein, or reduced phosphorylation of tyrosine residues in the Cbl protein (e.g. linked to growth receptor-mediated signalling events such as by measuring cAMP-mediated phosphorylation), or reduced binding of Cbl to an SH3-containing protein, would, indicate the ability of a compound to produce these effects. Standard assays for determining tyrosine phosphorylation of Cbl, receptor-mediated signalling of tyrosine phosphorylation in Cbl protein, ubiquitin ligase activity of Cbl or binding of Cbl to SH3 domains, are known to the skilled artisan.

Similarly, the ability of a compound to reduce feeding behavior, enhance fat deposition, reduce metabolic rate, or enhance the ratio of body fat to lean muscle mass can be assayed by determining whether or not the compound enhances ubiquitin ligase activity of Cbl protein, or enhances phosphorylation of tyrosine residues in the Cbl protein (e.g. linked to growth receptor-mediated signaling events such as by measuring cAMP-mediated phosphorylation), or enhances binding of Cbl to an SH3-containing protein.

By “Cbl-deficient” is meant that insufficient functional Cbl protein is produced to facilitate the level of glucose uptake or glycogen synthesis or lipogenesis detected in a wild type animal that does not suffer from a disorder of glucose metabolism. Glucose uptake, glycogen synthesis, or lipogenesis can readily be determined using known methods, such as, for example, by determining 2-deoxyglucose uptake into isolated liver, fat or muscle cells and/or glycogen synthesis and/or lipogenesis in the presence and absence of insulin as described by Lazar et al., J. Biol. Chem. 270: 20801-20807, 1995.

Preferably, the Cbl deficiency causes reduced Cbl expression at least in liver, fat or muscle cells of the genetically modified animal, more preferably in a tissue selected from the group consisting of adipose, skeletal muscle and cardiac muscle. Effects on Cbl expression in other cells or tissues, such as, for example, immune cells or brain, are not to be excluded from the scope of the present invention.

In one embodiment, the genetic modification capable of producing the Cbl deficient phenotype is selected from the group consisting of a deletion, an insertion, a substitution and an inversion of nucleotides in an allele of the Cbl locus. In another embodiment, the genetic modification is a deletion of a nucleotide sequence within two alleles of the Cbl locus, wherein the deletion results in an absence of expression of a functional or full-length Cbl protein by the animal. In a particularly preferred embodiment, the genetic modification comprises the deletion of a nucleotide sequence encoding a protein-encoding portion of the Cbl gene sufficient to prevent Cbl function, or alternatively, the introduction of an in-frame stop codon at a location in the protein-encoding portion of the Cbl gene sufficient to prevent expression of functional Cbl by one or both Cbl-encoding alleles. Even more preferably, the genetic modification comprises the targeted disruption of one or two alleles at the Cbl locus produces a truncated Cbl protein comprising the amino acid sequence set forth in SEQ ID NO: 1.

Preferably, the genetic modification is in both alleles of the Cbl locus (i.e. the animal is homozygous for the genetic modification).

The non-human animal is any mammal, such as, for example, a rodent, dog, pig, bovine, sheep, horse or goat. In one embodiment, the animal is a rodent selected from the group consisting of rabbit, rat, guinea pig and mouse. Conveniently, the animal is a mouse.

The Cbl-deficient “control” animal employed in this context can be any other animal that has reduced expression of Cbl expression and need not be isogenic to the animal on which the compound was tested (i.e. the “test” animal). Preferably, the control and test animals express similar levels of functional Cbl. More preferably, the control and test animals are isogenic. Preferably, the appetite or dietary intake of the animal is modified to a level that is comparable to the appetite or dietary intake of a wild-type animal and does not completely suppress appetite or dietary intake if the animal.

By “feeding behavior” is meant appetite or the amount of food consumption of the animal in a particular time interval, or its dietary intake expressed in absolute terms or alternatively, as a proportion of total body mass. Dietary intake will also be generally determined relative to a particular time interval (e.g. per hour, per day, etc). In most non-human animals, dietary intake is the preferred measure.

The range of compounds contemplated herein for modulating feeding behavior include peptides, including peptides derived from Cbl and capable of complementing the Cbl-deficiency; non-Cbl peptides, such as, for example Cbl peptidomimetics; small organic molecules, such as, for example derived from publicly available combinatorial libraries; and nucleic acids, including nucleic acid encoding said peptide derived from Cbl or said non-Cbl peptide.

In one embodiment, the subject method further comprises formulating the identified compound for administration to a non-human animal or a human. The formulations can be suitable for administration by injection by a subcutaneous, intravenous, intranasal, or intraperitoneal route. Alternatively, they can be suitable for oral administration in the form of feed additives, tablets, troches, etc.

The compounds are conveniently formulated in a suitable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such, as sodium chloride, Ringer's dextrose, etc. Preservatives include antimicrobial, anti-oxidants, chelating agents and inert gases. The pH and exact concentration of the various components the formulation suitable for administration to the animal are adjusted according to routine skills in the art. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such, as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Optionally, the formulation will also include a carrier, such as, for example, bovine serum albumin (BSA), keyhole limpet hemocyanin (KLH), ovalbumin, mouse serum albumin, rabbit serum albumin and the like. Means for conjugating peptides to carrier proteins are also well known in the art and include glutaraldehyde, m-maleimidobencoyl-N-hydroxysuccinimide ester, carbodiimide and bis-biazotized benzidine.

In another embodiment, the subject method further comprises producing or synthesizing the compound that is tested on the genetically modified animal.

Peptidyl compounds are conveniently made by standard peptide synthesis, such as the Merrifield method of synthesis (Merrifield, J Am Chem Soc, 85,:2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wünsch, E., ed. (1974) Synthese von Peptiden in Houben-Weyls Metoden der Organischen Chemie (Müler, E., ed.), vol. 15; 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.

Preferably, the peptide is synthesized on a solid phase support, such as, for example, a polystyrene gel bead comprising polystyrene cross-linked with divinylbenzene, preferably 1% (w.w) divinylbenzene, which is further swollen using lipophilic solvent, such as, for example dichloromethane or dimethylformamide (DMF). The polystyrene can be functionalized by addition of chloromethane or amino methyl groups. Alternatively, cross-linked and functionalized polydimethyl-acrylamide gel can be used once swollen and solvated using DMF or dipolar aprotic solvent. Other solid phase supports known to those skilled in the art can also be used for peptide synthesis, such as, for example, polyethylene glycol-derived bead produced by grafting polyethylene glycol to the surface of inert polystyrene beads. Preferred commercially available solid phase supports include PAL-PEG-PS, PAC-PEG-PS, KA, KR, or TGR (Applied Biosystems, CA 94404, USA).

For solid phase peptide synthesis, blocking groups that are stable to the repeated treatments necessary for removal of the amino blocking group of the growing peptide chain and for repeated amino acid couplings, are used for protecting the amino acid side-chains during synthesis and for masking undesired reactivity of the α-amino, carboxyl or side chain functional groups. Blocking groups (also called protecting groups or masking groups) thus protect the amino group of the amino acid having an activated carboxyl group that is involved in the coupling reaction, or protect the carboxyl group of the amino acid having an acylated amino group that is involved in the coupling reaction.

During synthesis, coupling occurs following removal of a blocking group without the disruption of a peptide bond, or any protecting group attached to another part of the peptide. Additionally, the peptide-resin-anchorage that protects the C-terminus of the peptide is protected throughout the synthetic process until cleavage from the resin is required. Accordingly, by the judicious selection of orthogonally protected α-amino acids, amino acids are added at desired locations to a growing peptide whilst it is still attached to the resin.

Preferred amino blocking groups are easily removable but sufficiently stable to survive conditions for the coupling reaction and other manipulations, such as, for example, modifications to the side-chain groups. In one embodiment, amino blocking groups are selected from the group consisting of: (i) a benzyloxycarbonyl group (Z or carbocenzoxy) that is removed easily by catalytic hydrogenation at room temperature and ordinary pressure, or using sodium in liquid ammonia and hydrobromic acid in acetic acid; (ii) a urethane derivative; (iii) a t-Butoxycarbonyl group (Boc) that is introduced using t-butoxycarbonyl azide or di-tert-butyldicarbonate and removed using mild acid such as, for example, trifluoroacetic acid (50% TFA in dichloromethane), or HCl in acetic acid/dioxane/ethylacetate; (iv) a 9-fluorenylmethyloxycarbonyl group (Fmoc) that is cleaved under mildly basic, non-hydrolytic conditions, such as, for example, using a primary or secondary amine (eg. 20% piperidine in dimethyl formamide); (v) a 2-(4-biphenylyl)propyl(2)oxycarbonyl group (Bpoc); (vi) a 2-nitro-phenylsulfenyl group (Nps); and (vii) a dithia-succionyl group (Dts). Boc is widely used to protect the N-terminus in Fmoc chemistry, or Fmoc is widely used to protect the N-terminus in Boc chemistry.

Side chain-protecting groups will vary for the functional side chains of the amino acids forming the peptide being synthesized. Side-chain protecting groups are generally based on the Bzl group or the tBu group. Amino acids having alcohols or carboxylic acids in the side-chain are protected as Bzl ethers, Bzl esters, cHex esters, tBu ethers, or tBu esters. Side-chain protection of Fmoc amino acids requires blocking groups that are ideally base stable and weak acid (TFA) labile. Many different protecting groups for peptide synthesis have been described (see The Peptides, Gross et al. eds., Vol. 3, Academic Press, New York, 1981). For example, the 4-methoxy-2,3,6-trimethylphenylsulfonyl (Nd-Mtr) group is useful for Arginine side-chain protection, however deprotection of Arg(Mtr), requires prolonged TFA treatment. A number of soft acid (TFA, thalium (III) trifluoroacetate/TFA) labile groups, or TFA stable but thalium (III) trifluoroacetate/TFA labile groups, or soft acid stable groups are used to protect Cystine.

The two most widely used protection strategies are the Boc/Bzl- and the Fmoc/tBu-strategies. In Boc/Bzl, Boc is used for amino protection and the side-chains of the various amino, acids are protected using Bzl- or cHex-based protecting groups. A Boc group is stable under catalytic hydrogenation conditions and is used orthogonally along with a Z group for protection of many side chain groups. In Fmoc/tBu, Fmoc is used for amino protection and the side-chains are protected with tBu-based protecting groups.

Alternatively, the peptidyl compound is produced by the recombinant expression of nucleic acid encoding the amino acid sequence of said peptide. Random peptide-encoding libraries are particularly preferred for such purposes, because they provide a wide range of different compounds to test. Alternatively, naturally-occurring nucleic acids can be screened. According to this embodiment, nucleic acid encoding the peptidyl compound is produced by standard oligonucleotide synthesis or derived from a natural source and cloned into a suitable expression vector in operable connection with a promoter or other regulatory sequence capable of regulating expression in a cell-free system or cellular system.

Oligonucleotides are preferably synthesized with linker or adaptor sequences at the 5′- and 3′-ends to facilitate subsequent cloning into a suitable vector system using standard techniques.

Placing a nucleic acid molecule under the regulatory control of, i.e., “in operable connection with”, a promoter sequence means positioning said molecule such that expression is controlled by the promoter sequence, generally by positioning the promoter 5′ (upstream) of the peptide-encoding sequence.

The prerequisite for producing intact peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake, and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, Calif.), amongst others.

Typical promoters suitable for expression in viruses of eukaryotic cells and eukaryotic cells include the SV40 late promoter, SV40 early promoter and cytomegalovirus (CMV) promoter, CMV-IE (cytomegalovirus immediate early) promoter, H1-RNA promoter, and U6 small nuclear RNA promoter, amongst others. Preferred vectors for expression in mammalian cells (eg., HeLa cells, HUVEC cells, 293 cells, 293 T cells, COS cells, COS-1 cells, CHO cells, CHO.T cells, C2C12 cells, differentiated 3T3-L1 adipocytes, 10T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing peptides in a secreted form in 293T cells, wherein the expressed peptide or protein can be purified free of conspecific proteins, using standard affinity techniques that employ a Nickel column to bind the protein via the His tag.

A wide range of additional host/vector systems suitable for expressing peptides are available publicly, and described, for example, in Sambrook et al (In: Molecular cloning, A laboratory manual, second edition, Cold Spring Harbor Laboratory, Cold Spring Harbor, N.Y., 1989).

Means for introducing the nucleic acid or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modelling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, information from known appetite suppressants and non-suppressants, including lead compounds identified in initial screens, can be used to expand the list of compounds being screened to thereby identify highly active compounds.

Another embodiment of the invention provides a method of identifying a compound that enhances feeding behavior, such as, for example, in the treatment of anorexia or bulimia, said method comprising: (a) administering a compound that suppresses appetite or dietary intake to a genetically modified non-human animal comprising a genetic modification within an allele of its Cbl locus wherein said genetic modification reduces or prevents expression of a functional endogenous Cbl in said animal and determining the feeding behavior of the animal; (b) administering a compound to the animal and determining the feeding behavior of the animal, wherein enhanced appetite or dietary intake at (b) compared to (a) indicates that the compound enhances feeding behavior.

Naturally, the compound administered at (b) will be different to the compound at (a).

Preferably, the compound that suppresses appetite or dietary intake acts via a Cbl-mediated mechanism, which can be verified using the genetically modified Cbl-deficient animal, such as by determining the ability of the compound to suppress appetite or dietary intake of Cbl-deficient mice according to a method described herein. In this case, step (a) supra of administering a compound that suppresses appetite or dietary intake to the genetically modified non-human animal will comprise: (i) administering a compound to a genetically modified non-human animal comprising a genetic modification within an allele of its Cbl locus wherein said genetic modification reduces or prevents expression of a functional endogenous Cbl in said animal; and (ii) determining the feeding behavior of the animal, wherein reduced appetite or dietary intake of the animal compared to a Cbl-deficient animal to which the compound has not been administered indicates that the compound suppresses appetite or dietary intake.

In an alternative embodiment, the invention provides a method of identifying a compound that modulates feeding behavior, such as, for example, in the treatment of anorexia or bulimia, said method comprising: (a) administering a compound to a non-human animal expressing a functional Cbl protein and determining the feeding behavior of the animal; (b) determining the feeding behavior of a genetically modified non-human animal comprising a genetic modification within an allele of its Cbl locus wherein said genetic modification reduces or prevents expression of a functional endogenous Cbl in said animal; and (c) comparing the feeding behavior of the animals at (a) and (b) wherein a comparable feeding behavior between (a) and (b) indicates that the compound modulates feeding behavior.

In one embodiment, the subject method further comprises formulating the identified compound for administration to a non-human animal or a human as described supra.

In another embodiment, the subject method further comprises producing or synthesizing the compound that is tested on the genetically modified animal.

The compounds contemplated include Cbl inhibitory compounds or antagonists of a biological function of Cbl. In one embodiment, the compound is selected from the group consisting of: a peptide, including a peptide derived from Cbl and capable of inhibiting, reducing or repressing a Cbl function, including binding to a protein selected from the group consisting of CAP, CrkII and C3G; a Cbl dominant-negative mutant; a non-Cbl peptide inhibitors of Cbl; an antibody or antibody fragment which binds to Cbl and inhibits a Cbl function; a small organic molecule, and nucleic acid, including nucleic acid encoding said peptide derived from Cbl or said non-Cbl peptide inhibitor, an antisense nucleic acid directed against Cbl-encoding mRNA, or an anti-Cbl ribozyme, or a small interfering RNA (RNAi) that targets Cbl gene expression.

The term “antisense nucleic acid” shall be taken to mean DNA or RNA molecule that is complementary to at least a portion of a specific mRNA molecule (Weintraub, Scientific American 262:40, 1990) and capable of interfering with a post-transcriptional event such as mRNA translation. Antisense oligomers complementary to at least about 15 contiguous nucleotides of Cbl-encoding mRNA are preferred, since they are easily synthesized and are less likely to cause problems than larger molecules when introduced into the target Cbl-producing cell. The use of antisense methods is well known in the art (Marcus-Sakura, Anal. Biochem. 172: 289, 1988). Preferred antisense nucleic acid will comprise a nucleotide sequence that is complementary to at least 15 contiguous nucleotides of a sequence encoding the amino acid sequence set forth in SEQ ID NO: 2 or 3.

As used herein, a “ribozyme” is a nucleic acid molecule having nuclease activity for a specific nucleic acid sequence. A ribozyme specific for Cbl-encoding mRNA, for example, binds tot and cleaves specific regions of the mRNA, thereby rendering it untranslatable. To achieve specificity, preferred ribozymes will comprise a nucleotide sequence that is complementary to at least about 12-15 contiguous nucleotides of a sequence encoding the amino acid sequence set forth in SEQ ID NO: 2 or 3.

As used herein, the terms “small interfering RNA” (“siRNA”), short hairpin RNA (“shRNA”), and “RNAi” refer to homologous double stranded RNA (dsRNA) that specifically targets a gene product, thereby resulting in a null or hypomorphic phenotype. Specifically, the dsRNA comprises two short nucleotide sequences derived from the target RNA encoding Cbl and having self-complementarity such that they can anneal, and interfere with expression of a target gene, presumably at the post-transcriptional level. RNAi molecules are described by Fire et al., Nature 391, 806-811, 1998, and reviewed by Sharp, Genes & Development, 13, 139-141, 1999). As will be known to those skilled in the art, short hairpin RNA (“shRNA”) is similar to siRNA, however comprises a single strand of nucleic acid wherein the complementary sequences are separated an intervening hairpin loop such that, following introduction to a cell, it is processed by cleavage of the hairpin loop into siRNA. Accordingly, each and every embodiment described herein is equally applicable to siRNA and shRNA.

Preferred siRNA or shRNA molecules comprise a nucleotide sequence that is identical to about 19-21 contiguous nucleotides of the target mRNA. Preferably, the target sequence in Cbl mRNA commences with the dinucleotide AA, comprises a GC-content of about 30-70% (preferably, 30-60%, more preferably 40-60% and more preferably about 45%-55%), and does not have a high percentage identity to any nucleotide sequence other than a c-Cbl protooncogene in the genome of the animal in which it is to be introduced, e.g., as determined by standard BLAST search.

The siRNA or shRNA is preferably capable of downregulating expression of human c-Cbl in a cell. In view of the high percentage conservation between murine and human c-Cbl-encoding genes, especially in the coding regions, this should not be taken to indicate a requirement for the siRNA or shRNA to be specific for human c-Cbl-encoding genes. In the cell-based and animal models described herein, it is possible and appropriate in certain circumstances for the siRNA or shRNA molecules to reduce expression of both endogenous murine c-Cbl, as well as ectopically expressed human c-Cbl in the cell. Confirmation of a specific activity of any antagonist against human c-Cbl is determined by assessing the activity of an inhibitor in a cell derived from a c-Cbl^{-/-} mouse that has been engineered to express human c-Cbl.

As exemplified herein, preferred siRNA against a c-Cbl encoding gene comprises a 21-nucleotide sequence set forth in any one of SEQ ID Nos: 4-239. In this respect, SEQ ID Nos: 4-121 each comprise (i) a 19-nucleotide sequence corresponding to a human c-Cbl mRNA target sequence adjacent and downstream of a dinucleotide AA in said mRNA target; and (ii) a 3′-extension dinucleotide TT. SEQ ID NOS: 122-239 each comprise (i) a 19-nucleotide sequence complementary to a human c-Cbl mRNA target sequence contained within SEQ ID NOS: 4-121, respectively; and (ii) a 3′-extension dinucleotide TT.

For producing shRNA from the exemplified siRNAs set forth in SEQ ID NOS: 4-239, the sense and antisense strands are positioned such that they flank an intervening loop sequence. Preferred loop sequences are selected from the group consisting of:

| | | |
|---|---|---|
| (i) | CCC; | (SEQ ID NO:240) |
| (ii) | TTCG; | (SEQ ID NO:241) |
| (iii) | CCACC; | (SEQ ID NO:242) |
| (iv) | CTCGAG; | (SEQ ID NO:243) |
| (v) | AAGCTT; | (SEQ ID NO:244) |
| (vi) | CCACACC; | (SEQ ID NO:245) |
| and | | |
| (vii) | TTCAAGAGA. | (SEQ ID NO:246) |

Of these loop sequences, the sequence set forth in SEQ ID NO: 246 is particularly preferred for modulating human c-Cbl expression in a cell, tissue (eg., skeletal muscle, or whole organism).

Preferred siRNA molecules that are selectively active against human c-Cbl expression compared to murine c-Cbl expression are derived from the sequence of the 5′-non-coding and/or 3′-non-coding region of the human c-Cbl gene. Such specific siRNAs include, e.g., SEQ ID Nos: 57-59, 117-121, 175-177 and 235-239.

The antisense RNA, ribozyme, siRNA or shRNA can be introduced directly to a cell or cell-free extract capable of expressing c-Cbl as naked DNA. Alternatively, DNA encoding a nucleic acid inhibitory molecule can be introduced into a cell in operable connection with a suitable promoter and transcription terminator sequence to facilitate expression of the inhibitory nucleic acid. Preferred promoters for expression in mammalian cells that express Cbl include the CMV promoter, ubiquitin promoter, U6 small nuclear RNA promoter (Lee et al., Nature Biotech. 20, 500-505, 2002; Miyagishi et a;., Nature Biotech 20, 497-500, 2002; Paul et al., Nature Biotech. 20, 505-508, 2002; and Yu et. al., Proc. Natl Acad. Sci USA 99, 6047-6052, 2002), H1-RNA promoter (Brummelkamp et al., Science 296, 550-553, 2002), or other RNA polymerase III promoter; The pot III terminator is also preferred for such applications. Other promoters and terminators are not to be excluded.

In one embodiment, the DNA encoding the inhibitory nucleic acid is operably connected to promoter and terminator regulatory sequences by cloning into a suitable vector that comprises the necessary promoter and transcriptional terminator sequences, and the recombinant vector is then introduced to the cell, tissue or organ by transient transfection of plasmid DNA, by establishing permanent cell lines or in infection with retroviral expression vectors (Barton et al., Proc. Natl. Acad. Sci USA 99, 14943-14945, 2002; Devroe et al., BMC Biotech. 2, p 15, 2002).

In high throughput primary assays at least, it is preferred to use an in vitro cell-free system or cell-based system in which Cbl activity is assayed. Several vectors are known for this purpose, including, for example, the pSilencer series of vectors (pSilencer 2.0, pSilencer 2.1, pSilencer 3.0, pSilencer 3.1, pSilencer 1.0-U6) provided by Ambion.

Preferred retroviral vectors, suitable for transiently transfecting into isolated cells e.g., by calcium phosphate precipitation (Ketteler et al., Gene Ther. 9, 477-487, 2002) in high throughput screens, or for the production of transducing supernatants (Ketteler et al., Gene Ther. 9, 477-487, 2002) for lower-throughput screening or validation of primary screen results, include pBABE (Morgenstern et al., Nuc. Acids Res. 18, 3587-3596, 1990) and JZenNeo.

The pBabe retroviral vector constructs transmit inserted genes at high titres and express them from the Mo MuLV Long Terminal Repeat (LTR). The pBabe vectors comprise one of four different dominantly-acting selectable markers, allowing the growth of infected mammalian cells in the presence of G418, hygromycin B, bleomycin/phleomycin or puromycin. The high titre ecotropic helper free packaging cell line, omega E, reduces the risk of generation of wild type Mo MuLV via homologous recombination events. Together, the pBabe vectors and omega E cell line provide high frequency gene transfer, and/or concomitant expression of c-Cbl with one or more other genes (e.g., APS and/or Insulin receptor β-subunit) in a single cell, with minimal risk of helper virus contamination.

For lower throughput primary screening or validation assays, the adenoviral vectors pAdTrack and pAdTrack-CMV (He et al., Proc. Natl Acad. Sci USA 95, 2509-2514, 1998; pAdTrack-HP (Zhao et al., Gene 316, 137-141, 2003), an Ad5CMV-based vector e.g., Ad5CMV-GFP (Suoka et al., Am. J. Respir. Cell Mol. Biol. 23, 297-303, 2000), and pSilencer adeno 1.0-CMV (Ambion) are preferred for delivery and expression in specific organs or tissues, in particular muscle tissue of a mouse model.

The pAdTrack and pAdTrack-CMV vectors are particularly preferred for applications which require standardization for transfection or transduction efficiency eg., injection of adenovirus into hindlimb muscles of transgenic mouse models. The pAdTrack vector is used for production of GFP-trackable viruses containing transgenes under the control of a chosen promoter. It contains the gene encoding enhanced GFP, a polylinker for insertion of exogenous transgenes surrounded by adenoviral sequences (“arms”) that allow homologous recombination with pAdEasy-1. The left arm contains Ad5 nucleotides 34,931-35,935, which mediate homologous recombination with pAdEasy vectors in E. coli, plus inverted terminal repeat (ITR) and packaging signal sequences (nucleotides 1-480 of Ad5) required for viral production in mammalian cells. The right arm contains Ad5 nucleotides 3,534-5,790, which mediate homologous recombination with pAdEasy vectors. Artificially created PacI sites surround both arms. The AdTrack plasmid also contains a kanamycin resistance gene from pZero 2.1 (Invitrogen) and the origin of replication from pBR322 (Life Technologies). The relatively low copy number of plasmids generated with this origin is essential for the stability of large constructs in E. coli. The pAdTrack-CMV vector is identical to pAdTrack except for the addition of a cytomegalovirus (CMV) promoter and polyadenylation site (both from pEGFP-C1, Clontech). A polylinker is present between the CMV promoter and polyadienylation site.

As will be known to the skilled artisan, such adenoviral vectors are also suitable for transfection of cell lines.

The term “dominant-negative mutant” refers to a Cbl polypeptide that has been mutated from its natural state and that interacts with a protein that Cbl normally interacts with thereby preventing endogenous native Cbl from forming the interaction. Preferred dominant negative mutants will lack that portion of Cbl that interacts with a protein selected from the group consisting of CAP, CrkII and C3G.

Preferred dominant-negative mutants comprise variants of the native Cbl protein, such as, for example, substitution or deletion mutants, that act as dominant-negative mutants of Cbl function. For example, a dominant-negative mutant may comprise one or more amino acid substitutions within the RING domain of c-Cbl. When expressed in a cell, such a dominant-negative mutant protein competes with native endogous c-Cbl for the APS protein and/or insulin receptor β-subunit, however has reduced or no E3 ubiquitin ligase activity with respect to the receptor. In an alternative embodiment, the dominant-negative mutant comprises 1 or 2 or 3 substitutions of tyrosine residues that would normally be phosphorylated in the cell. Means for producing mutated nucleic acid are well known to those skilled in he art and may be achieved readily e.g., using the Quick Change Mutagenesis kit supplied by Stratagene, La Jolla, Calif. USA according to the manufacturer's instructions.

Dominant negative mutant proteins are produced by expression of nucleic acid encoding the mutant protein, essentially as described herein above for expression of peptides in cells. The amino acid sequences of exemplary dominant-negative mutants of c-Cbl are set forth in SEQ ID NO: 248 (c-Cbl G306E), SEQ ID NO: 250 (c-Cbl C381A), SEQ ID NO: 252 (c-Cbl Y700F), SEQ ID NO: 254 (c-Cbl Y731F), SEQ ID NO: 256 (c-Cbl Y774F), SEQ ID NO: 258 (c-Cbl Y700F/Y731F/Y774F) and SEQ ID NO: 260 (c-Cbl 480 i.e, truncated at residue 480 of native human c-Cbl).

The “antibodies” contemplated herein are immunoreactive with Cbl polypeptides or functional fragments thereof. Antibodies that consist essentially of pooled monoclonal antibodies with different epitope specificities, as well as distinct monoclonal antibody preparations are contemplated. Monoclonal antibodies are produced from fragments of the Cbl protein that comprise one or more B cell epitopes by methods well known to those skilled in the art (Kohler et al, Nature 256:495, 1975). The term “antibody” as used herein includes intact molecules as well as fragments thereof, such as Fab and F(ab′)₂, Fv and single chain antibody fragments capable of binding an epitopic determinant of Cbl.

An “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain.

An “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An “F(ab′)₂ fragment” of an antibody consists of a dimer of two Fab′ fragments held together by two disulfide bonds, and is obtained by treating a whole antibody molecule with the enzyme pepsin, without subsequent reduction. A (Fab′).sub.2 fragment.

An “Fv fragment” is a genetically engineered fragment containing the variable region of a light chain and the variable region of a heavy chain expressed as two chains.

A “single chain antibody” (SCA) is a genetically engineered single chain molecule containing the variable region of a light chain and the variable region of a heavy chain, linked by a suitable, flexible polypeptide linker.

In one embodiment, peptidyl Cbl inhibitors are chemically or recombinantly synthesized as oligopeptides (approximately 10-25 amino acids in length) spanning the Cbl sequence (SEQ ID NO: 2 or 3). Alternatively, Cbl fragments are produced by digestion of native or recombinantly produced Cbl by, for example, using a protease, e.g., trypsin, thermolysin, chymotrypsin, or pepsin. Computer analysis (using commercially available software, e.g. MacVector, Omega, PCGene, Molecular Simulation, Inc.) is used to identify proteolytic cleavage sites. The proteolytic or synthetic fragments can comprise as many amino acid residues as are necessary to partially or completely inhibit Cbl function. Preferred fragments will comprise at least 5, 10, 15, 20, 25, 30, 35, 40, 45, 50, 55, 60, 65, 70, 75, 80, 85, 90, 95, 100 or more amino acids in length.

In one embodiment, peptides are selected which contain a sufficient number of B cell epitopes to elicit antibodies when administered to a mammal. Such peptides are identified by immunizing a mammal with the peptide alone, or in combination with an adjuvant, or linked to an adjuvant (e.g., a hapten). Sera from the immunized animal are tested for anti-Cbl antibodies. Preferred peptides generate anti-Cbl antibodies that inhibit a Cbl function.

Preferred peptidyl Cbl inhibitors will also not comprise a sufficient number of T cell epitopes to induce T-cell mediated (e.g., cytokine) responses when determined using any of a number of well known techniques, such as epitope prediction using algorithms (see e.g., Rothbard and Taylor EMBO J. 7: 93-100, 1988; Berzofsky, Philos Trans R. Soc. Lond. 323: 535-544, 1989; Rothbard, 1st Forum in Virology, Annals of the Pasteur Institute, pp 518-526, December 1986; Rothbard and Taylor, Embo, 7: 93-100, 1988; EP 0 304 279; and Margalit et al., J. Immunol., 138: 2213-2229, 1987); or screening of peptide inhibitors for human T cell stimulating activity or T cell proliferation assays (e.g. Proc. Natl. Acad. Sci USA, 86:1333, 1989).

Other preferred peptide inhibitors of Cbl are located on the surface of the Cbl proteins, e.g., hydrophilic regions, as well as regions with high antigenicity or fragments with high surface probability scores can be identified using computer analysis programs well known to those of skill in the art (Hopp and Wood, (1983), Mol. Immunol., 20, 483-9, Kyte and Doolittle, (1982), J. Mol. Biol., 157, 105-32, Corrigan and Huang, (1982), Comput. Programs Biomed, 3, 163-8).

Anti-Cbl antibodies or antibody fragments are generated using the entire Cbl polypeptide or an immunogenic fragment thereof (alone or linked to a suitable carrier or hapten) to immunize a subject (e.g., a mammal including, but not limited to a rabbit, goat, mouse or other mammal). For example, the methods described in U.S. Pat. Nos. 5,422,110; 5,837,268; 5,708,155; 5,723,129; and 5,849,531, can be used and are incorporated herein by reference. In a preferred embodiment, the mammal being immunized does not contain endogenous Cbl (e.g., a Cbl-deficient genetically modified animal). The immunogenic preparation can further include an adjuvant, such as Freund's complete or incomplete adjuvant, or similar immunostimulatory agent. Immunization of a suitable subject with an immunogenic proteolytic or synthetic Cbl peptide preparation induces a polyclonal anti-Cbl antibody response. The anti-Cbl antibody titer in the immunized subject is generally monitored over time by standard techniques, such as with an enzyme linked immunosorbent assay (ELISA) using immobilized Cbl. Subsequently, the sera from the immunized subjects can be tested for Cbl inhibitory activity.

Alternatively, it is also possible to immunize the subject with nucleic acid expressing Cbl using DNA immunization technology, such as that disclosed in U.S. Pat. No. 5,795,872 to Ricigliano et al., or alternatively, in U.S. Pat. No. 5,643,578 to Robinson et al.

The antibody molecules directed against Cbl can be isolated from the mammal (e.g., from the blood) and further purified by well known techniques, such as protein A chromatography to obtain the IgG fraction. At an appropriate time after immunization, e.g., when the anti-Cbl antibody titers are highest, antibody-producing cells can be obtained from the subject and used to prepare, e.g., monoclonal antibodies by standard techniques, such as the hybridoma technique originally described in the following disclosures: Kohler and Milstein Nature 256:495-497, 1975; Brown et al. J. Immunol. 127:53946, 1981; Brown et al; J. Biol Chem. 255: 4980-4983, 1980; Yeh et al. Proc. Natl. Acad. Sci. USA 76:2927-2931, 1976; Yeh et al. Int. J. Cancer 29: 269-275, 1982; Kozbor et al, Immunol Today 4:72, 1983; Cole et al., Monoclonal Antibodies and Cancer Therapy, Alan R. Liss, Inc., pp. 77-96, 1985. The technology for producing monoclonal antibody hybridomas is well known in the art. Briefly, an immortal cell line (typically a myeloma) is fused to lymphocytes (typically splenocytes) from a mammal immunized with a Cbl peptide immunogen as described above, and the culture supernatants of the resulting hybridoma cells are screened to identify a hybridoma producing a monoclonal antibody that binds Cbl.

Any of the known protocols used for fusing lymphocytes and immortalized cell lines can be applied for the purpose of generating an anti-Cbl monoclonal antibody (see, e.g., G. Galfre et al., Nature 266: 550-552, 1970). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Cbl, e.g., using a standard ELISA assay. The antibodies can then be tested for Cbl inhibitory activity.

In one embodiment, the present invention a small organic molecule inhibitor or antagonist of c-Cbl is a compound that reduces the E3 ubiquitin ligase activity of c-Cbl thereby reduces the ubiquitination of the insulin receptor β-subunit by c-Cbl. Preferably, the small organic molecule is a non-competitive inhibitor of c-Cbl with respect to ubiquitin and the insulin receptor β-subunit. In a preferred embodiment, the small organic molecule belongs to a class of compounds that binds to the RING finger domain of c-Cbl (i.e. an amino acid sequence comprising or contained within residues from about position 380 to about position 421 of SEQ ID NO: 3), e.g., a benzsulfonamide, urea, or imidazolone.

A further aspect of the present invention provides a method of identifying a compound that enhances fat deposition or reduces lean muscle mass or enhances the ratio of body fat to muscle or reduces metabolic rate, such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a genetically modified non-human animal comprising a genetic modification within an allele of its Cbl locus wherein said genetic modification reduces or prevents expression of a functional endogenous Cbl in said animal; and (b) determining the fat content of the animal, wherein enhanced fat content of the animal compared to the fat content of a Cbl-deficient animal to which the compound has not been administered indicates that the compound enhances fat deposition or reduces lean muscle mass or enhances the ratio of body fat to muscle or reduces metabolic rate.

By “fat deposition” is meant the amount of fat deposited or the rate at which fat is deposited in liver, fat or muscle cells. Standard means known to the skilled artisan are used to determine fat deposition. “Lean muscle mass” means muscle tissue that is substantially fat-free. The “ratio of body fat to muscle mass” means the relative amount of total body fat to total non-fat tissue. “Metabolic rate” means the ability of a subject to utilize dietary intake for immediate energy needs, rather than store such dietary intake as body fat.

The genetically modified animal and the Cbl-deficient animal are described supra.

The range of compounds contemplated herein for enhancing fat deposition or reducing lean muscle mass or enhancing the ratio of body fat to muscle or reducing metabolic rate include peptides, including peptides derived from Cbl and capable of complementing the Cbl-deficiency; non-Cbl peptides, such as, for example Cbl peptidomimetics; small organic molecules, such as, for example derived from publicly available combinatorial libraries; and nucleic acids, including nucleic acid encoding said peptide derived from Cbl or said non-Cbl peptide. Such compounds are described supra.

In one embodiment, the subject method further comprises formulating the identified compound for administration to a non-human animal or a human. Such formulations are described supra.

In another embodiment, the subject method further comprises producing or synthesizing the compound that is tested on the genetically modified animal. Such methods are described supra.

In an alternative embodiment, the invention provides a method of identifying a compound that reduces fat deposition or enhances lean muscle mass or reduces the ratio of body fat to muscle or enhances metabolic rate, such as, for example, in the treatment of obesity, said method comprising: (a) administering a compound that enhances fat deposition or glucose uptake to a genetically modified non-human animal comprising a genetic modification within an allele of its Cbl locus wherein said genetic modification reduces or prevents expression of a functional endogenous Cbl in said animal and determining the fat content of the animal; (b) administering a compound to the animal and determining the fat content of the animal, wherein a similar or reduced fat content at (b) compared to (a) indicates that the compound reduces fat deposition or enhances lean muscle mass or reduces the ratio of body fat to muscle or enhances metabolic rate.

Preferably, the animal is, maintained on a diet comprising high glycemic index food, such as, for example, carrots or food supplemented with sucrose.

Preferably, the compound that enhances fat deposition, or reduces lean muscle mass or enhances the ratio of body fat to muscle or reduces metabolic rate acts via a Cbl-mediated mechanism, which can be verified using the genetically modified Cbl-deficient animal, such as by determining the ability of the compound to enhance fat content of Cbl-deficient mice according to a method described herein. In this case, step (a) supra of administering a compound that enhances fat deposition or glucose uptake comprises (i) administering a compound to a genetically modified non-human animal comprising a genetic modification within an allele of its Cbl locus wherein said genetic modification reduces or prevents expression of a functional endogenous Cbl in said animal; and (ii) determining the fat content of the animal, wherein enhanced fat content of the animal compared to a Cbl-deficient animal to which the compound has not been administered indicates that the compound enhances fat deposition, or reduces lean muscle mass or enhances the ratio of body fat to muscle or reduces metabolic rate.

In an alternative embodiment, the invention provides a method of identifying a compound that reduces fat deposition or enhances lean muscle mass or reduces the ratio of body fat to muscle or enhances metabolic rate comprising: (a) administering a compound to a non-human animal expressing a functional Cbl protein and determining the fat content of the animal; (b) determining the fat content of a genetically modified non-human animal comprising a genetic modification within an allele of its Cbl locus wherein said genetic modification reduces or prevents expression of a functional endogenous Cbl in said animal; and (c) comparing the fat contents of the animals at (a) and (b) wherein a comparable fat content between (a) and (b) indicates that the compound reduces fat deposition or enhances lean muscle mass or reduces the ratio of body fat to muscle or enhances metabolic rate.

Preferably, the animals are maintained on similar or identical diets, more preferably, on diets comprising high glycemic index food.

The compounds contemplated herein include Cbl inhibitory compounds or antagonists of a biological function of Cbl. In one embodiment, the compound is selected from the group consisting of: a peptide, including a peptide derived from Cbl and capable of inhibiting, reducing or repressing a Cbl function, including binding to a protein selected from the group consisting of CAP, CrkII and C3G; a Cbl dominant-negative mutant; a non-Cbl peptide inhibitors of Cbl; an antibody or antibody fragment which binds to Cbl and inhibits a Cbl function; a small organic molecule, and nucleic acid, including nucleic acid encoding said peptide derived from Cbl or said non-Cbl peptide inhibitor, an antisense nucleic acid directed against Cbl-encoding mRNA, an anti-Cbl ribozyme, RNAi or siRNA.

In one embodiment, the subject method further comprises formulating the identified compound for administration to a non-human animal or a human. Such formulations are described supra.

In another embodiment, the subject method further comprises producing or synthesizing the compound that is tested on the genetically modified animal. Such methods are described supra.

A further aspect of the present invention provides a method of identifying a compound that enhances glucose uptake such as, for example, in the treatment of hypolipidemia (e.g. as observed in subjects suffering from abetalipoproteinemia, malnutrition or hematologic malignancies, such as acute myelocytic leukemia or chronic myelocytic leukemia), said method comprising: (a) administering a compound to a genetically modified non-human animal comprising a genetic modification within an allele of its Cbl locus wherein said genetic modification reduces or prevents expression of a functional endogenous Cbl in said animal; and (b) determining the glucose uptake into liver, fat or muscle cells of the animal, wherein enhanced uptake compared to the glucose uptake into liver, fat or muscle cells of a Cbl-deficient animal to which the compound has not been administered indicates that the compound enhances glucose uptake.

Means for determining glucose uptake are well known in the art. Preferably, the process is performed ex vivo using liver, fat or muscle cells that have been previously isolated from the animal. In one embodiment, the glucose uptake is basal glucose uptake (i.e. glucose uptake measured in the absence of exogenously administered insulin). In another embodiment, the glucose uptake is insulin-mediated glucose uptake (i.e. glucose uptake measured following administration of insulin). All embodiments of the invention described herein apply mutatis mutandis to both basal glucose uptake and insulin-mediated glucose uptake unless otherwise stated.

The genetically modified animal and the Cbl-deficient animal are described supra.

The range of compounds contemplated herein for enhancing glucose uptake include peptides, including peptides derived from Cbl and capable of complementing the Cbl-deficiency; non-Cbl peptides, such as, for example Cbl peptidomimetics; small organic molecules, such as, for example derived from publicly available combinatorial libraries; and nucleic acids, including nucleic acid encoding said peptide derived from Cbl or said non-Cbl peptide. Such compounds are described supra.

In one embodiment, the subject method further comprises formulating the identified compound for administration to a non-human animal or a human. Such formulations are described supra.

In another embodiment, the subject method further comprises producing or synthesizing the compound that is tested on the genetically modified animal. Such methods are described supra.

In an alternative embodiment, the invention provides a method of identifying a compound that reduces glucose uptake into liver, fat or muscle cells, such as, for example, in the treatment of obesity, said method comprising: (a) administering a compound that enhances glucose uptake to a genetically modified non-human animal comprising a genetic modification within an allele of its Cbl locus wherein said genetic modification reduces or prevents expression of a functional endogenous Cbl in said animal and determining the glucose uptake into liver, fat or muscle cells; (b) administering a compound to the animal and determining the glucose uptake into liver, fat or muscle cells of the animal, wherein a similar or reduced uptake at (b) compared to (a) indicates that the compound reduces glucose uptake into liver, fat or muscle cells.

Preferably, the animal is maintained on a diet comprising high glycemic index food, such as, for example, carrots or food supplemented with sucrose.

Preferably, the compound that enhances glucose uptake into liver, fat or muscle cells acts via a Cbl-mediated mechanism, which can be verified using the genetically modified Cbl-deficient animal, such as by determining the ability of the compound to enhance glucose uptake into liver, fat or muscle cells of Cbl-deficient mice according to a method described herein. In this case, step (a) supra of administering a compound that enhances glucose uptake into liver, fat or muscle cells comprises (i) administering a compound to a genetically modified non-human animal comprising a genetic modification within an allele of its Cbl locus wherein said genetic modification reduces or prevents expression of a functional endogenous Cbl in said animal; and (ii) determining the glucose uptake into liver, fat or muscle cells of the animal, wherein enhanced glucose uptake into liver, fat or muscle cells of the animal compared to a Cbl-deficient animal to which the compound has not been administered indicates that the compound enhances glucose uptake into liver, fat or muscle cells.

In an alternative embodiment, the invention provides a method of identifying a compound that reduces glucose uptake into liver, fat or muscle cells comprising: (a) administering a compound to a non-human animal expressing a functional Cbl protein and determining the glucose uptake into liver, fat or muscle cells of the animal; (b) determining the glucose uptake into liver, fat or muscle cells of a genetically modified non-human animal comprising a genetic modification within an allele of its Cbl locus wherein said genetic modification reduces or prevents expression of a functional endogenous Cbl in said animal; and (c) comparing the glucose uptake into liver, fat or muscle cells of the animals, at (a) and (b) wherein a comparable uptake between (a) and (b) indicates that the compound reduces glucose uptake into liver, fat or muscle cells.

Preferably, the animals are maintained on similar or identical diets, more preferably, on diets comprising high glycemic index food.

The compounds contemplated herein include Cbl inhibitory compounds or antagonists of a biological function of Cbl. In one embodiment, the compound is selected from the group consisting of: a peptide, including a peptide derived from Cbl and capable of inhibiting, reducing or repressing a Cbl function, including binding to a protein selected from the group consisting of CAP, CrkII and C3G; a Cbl dominant-negative mutant; a non-Cbl peptide inhibitors of Cbl; an antibody or antibody fragment which binds to Cbl and inhibits a Cbl function; a small organic molecule, and nucleic acid, including nucleic acid encoding said peptide derived from Cbl or said non-Cbl peptide inhibitor, an antisense nucleic acid directed against Cbl-encoding mRNA, or an anti-Cbl ribozyme.

In one embodiment, the subject method further comprises formulating the identified compound for administration to a non-human animal or a human. Such formulations are described supra.

In another embodiment, the subject method further comprises producing or synthesizing the compound that is tested on the genetically modified animal. Such methods are described supra.

In an alternative embodiment, the screening assays supra are performed in vitro using a format that determines the E3 ubiquitin ligase activity of Cbl. Such assays are based upon the knowledge that the APS adapter protein couples the precursor insulin receptor β-subunit to the phosphorylation of c-Cbl and facilitates ligand-stimulated ubiquitination of the insulin receptor (IR-β) (Ahmed et al., FEBS Letts 475, 31-34, 2000). Accordingly, to determine ubiquitination of the insulin receptor by c-Cbl, cells that either express APS, c-Cbl and IR-β are required. CHO.T cells are particularly preferred for this purpose, because they are known to over express the IR-β precursor protein.

In one embodiment, a test sample is contacted with a test compound under suitable test conditions, wherein the test sample comprises APS protein, c-Cbl protein, insulin receptor (IR-β) protein, an El enzyme, a ubiquitin conjugating enzyme, ubiquitin and adenosine 5′-triphosphate (ATP). The ubiquitination of IR-β is determined and the level of ubiquitination of IR-β in the test sample with the ubiquitination of IR-β incubated under the same test conditions in the absence of test compound is determined. The level of ubiquitination of IR-β in the test sample is indicative of the ability of the test compound to modulate ubiquitin ligase activity of c-Cbl. An increase in the level of ubiquitination is an indication that ubiquitin ligase activity of c-Cbl has been enhanced, whereas a decrease in the level of ubiquitination is an indication that ubiquitin ligase activity of c-Cbl has been inhibited.

E1 enzyme is well known to one of skill in the art (e.g., Hershko et al., Ann. Rev. Biochem. 61:761-807, 1992, and Monia et al., Biotechnol. 8: 209-215, 1990, herein incorporated by reference). E1 enzyme initiates the ubiquitination process by activating ubiquitin. Any of the E1 enzymes known in the art are suitable for use.

Ubiquitin conjugating enzymes transfer ubiquitin to lysine residues of suitable substrates. They also undergo auto-ubiquitination. Suitable ubiquitin conjugating enzymes that can be employed in the invention method include Cdc34, UbcH1, UbcH2, UbcH3, UbcH4, UbcH5, UbcH6, UbcH7, UbcH10, L-UBC, and the like (see Kaiser, et al, FEBS Letts 350:1-4, 1994; Kaiser, et al, FEBS Letts 377:193-196, 1995; Nuber, et al, J Biol Chem 271:2795-2800, 1996; Jensen, et al, J Biol Chem 270:30408-30414, 1995; Robinson, et al, Mamm Genome 6:725-731, 1995; and Plon et al., Proc. Natl. Acad. Sci. USA 90:10484-10488, all of which are herein incorporated by reference). “Cdc34” refers to a ubiquitin-conjugating enzyme isolated from yeast.

In one embodiment, the ubiquitin is a derivatized ubiquitin conjugated to a label that is readily identified. For example, the derivatized ubiquitin can be [¹²⁵I]-ubiquitin, a fluorescent ubiquitin, a glutathione S-transferase conjugated ubiquitin or a biotinylated ubiquitin. Using assays well known in the art, the presence of the label, and thus the amount of derivatized ubiquitination, can be identified.

Ubiquitination results in an increase in the molecular weight of the IR-β polypeptide. Accordingly, any assay that determines the molecular weight of the IR-β polypeptide, such as, for example, SDS-polyacrylamide gel electrophoresis, mass spectrometry or a variant thereof (MALDI, MALDI-TOF, MS/MS, etc), can be used to measure ubiquitination. High throughput procedures, such as, for example, MS/MS and MALDI-TOF are particularly preferred.

The ubiquitination assay is readily adapted to the large scale screening of compound libraries by converting it to a solid phase format. In one specific non-limiting example, ubiquitination assays can be performed with an appropriately engineered chimeric IR-β protein immobilized on a microtiter plate, in the presence of a derivatized ubiquitin. A “chimeric IR-β protein” in this context is an IR-β precursor polypeptide comprising a heterologous peptide or polypeptide. Preferred heterologous peptides in this context include a myc epitope-hexahistidine tag positioned at the C-terminus of IR-β (i.e., IR-β-myc-His₆).

In one embodiment of high throughput screening for ubiquitination of IR-β, components of the reaction mixture are contacted, and aliquots of the ubiquitination assays are transferred to a microtiter plate with an appropriate surface to permit binding of IR-β protein thereto. Preferably, reactions are transferred to a microtiter plate whose wells have been coated with anti-IR-β antibody or anti-myc antibody. After washing away unbound proteins, the substrate-coated wells are directly imaged (e.g., for reactions performed with fluorescent or radio-labeled ubiquitin). The assay may be conducted in replica plates e.g., in the presence or absence of a series of compounds being tested.

In an alternative embodiment, ubiquitination of the IR-β substrate in the presence of a compound is determined using a cell-based assay. The only requirement for such an assay is that the compound being tested is capable of entering the cell. Accordingly, such an assay is particularly useful for identifying peptidyl and nucleic acid modulatory compounds that can be efficiently expressed following transfection or transduction into cells e.g., ribozymes, antisense, siRNA, shRNA, dominant negative modulatory. Such assays may also be amenable for determining the efficacy of antibodies or small organic molecules. This embodiment is based upon the finding that detectable c-Cbl-mediated ubiquitination of IR-β precursor occurs in CHO.T cells expressing myc-tagged APS (Ahmed et al., FEBS Letts 475, 31-34, 2000).

In one embodiment, cells capable of expressing APS, c-Cbl and IR-β precursor are incubated in the presence ubiquitin or a derivatized ubiquitin and a chemical compound to be tested for a time and under conditions sufficient for c-Cbl-mediated ubiquitination of IR-β precursor to occur, and the amount of ubiquitin bound to the IR-β precursor is determined.

In contrast to the cell-free system described supra, specific detection of ubiquitinated IR-β precursor is required, because other proteins will be ubiquitinated in the cells. Accordingly, it is particularly, preferred to determine the amount of ubiquitin bound to IR-β precursor by first capturing the insulin receptor holoprotein or IR-β precursor polypeptide with an antibody (e.g., anti-insulin receptor antibody 83-14, Abcsam, Inc, Cambridge Mass. 02139, USA; or specific anti-IR-β antibody as supplied e.g., by Lab Vision Corp., Fremont Calif. 94539, USA) and then detecting the amount of ubiquitin bound thereto using a second antibody that specifically binds ubiquitin (Santa Cruz Biotechnology, Santa Cruz, Calif., USA). As with the cell-free assay system supra, a derivatized ubiquitin may be imaged directly.

In a particularly preferred embodiment, antibodies that bind the insulin receptor are immobilized on a solid substrate, e.g., wells of a microtiter plate and contacted with cell extracts for a time and under conditions sufficient for an antigen-antibody complex to form thereby capturing the insulin receptor. After washing away unbound proteins, the wells are either directly imaged (e.g., for reactions performed with fluorescent or radio-labeled ubiquitin) or contacted with anti-ubiquitin antibody for a time and under conditions sufficient for an antigen-antibody complex to form. Binding of the anti-ubiquitin antibody is detected using a second antibody conjugated to horse-radish peroxidase enzyme or other detectable label.

The assay may be conducted in replica plates e.g., in the presence or absence of a series of compounds being tested to facilitate comparison of the amount of ubiquitination of the receptor in the presence and absence of the compound being tested.

In a further embodiment, a first replica plate is contacted with second antibody after capturing the insulin receptor and a second replica plate is contacted with a second antibody after binding of the anti-ubiquitin antibody. In accordance with this embodiment, measurement of the ratio of the amount of second antibody bound to the first and second replica plates indicates the amount of ubiquitinated insulin receptor relative to the total amount of insulin receptor in the cell or sample. Such measurement is particularly useful for correcting for sample variations. To facilitate high throughput analyses, it is particularly preferred for the second antibodies to be labelled with different colored dyes or fluorophores to permit their simultaneous detection.

A further aspect of the present invention provides-methods for determining a modulator of the activity, formation or stability of a protein complex selected from the group consisting of: (i) a Cbl-APS complex; (ii) a Cbl-CAP complex; (iii) a Cbl-CAP-flotillin complex; (iv) a Cbl-C3G complex; (v) a Cbl-CrkII complex; and (vi) a Cbl-C3G-CrkII complex.

In their general form, the methods of the present invention comprise determining the association or dissociation of the protein complex, or the structure of the complex, in the presence and absence of a candidate compound or a candidate antibody. In accordance with the embodiment described herein, a modified association, dissociation, or structure, of the protein complex in the presence of a candidate compound or a candidate antibody indicates that the candidate is a modulator of the protein complex.

The association, dissociation, or structure of the complex may be determined by direct means, such as, for example, by determining real time association or dissociation constants in the presence and absence of the candidate, or modified binding of an antibody that recognizes a conformational epitope of the complex. Biosensors used essentially as described herein above, in the presence or absence of the candidate compound or antibody, are particularly suited to such applications.

Alternatively, the association, dissociation, or structure of the complex may be determined by indirect means, such as, for example, using a protein recruitment system, n-hybrid screen, reverse n-hybrid screen, plate agar diffusion assay, ELISA, or other well known assay format for detecting protein-protein interactions. Such indirect means generally use a reporter system to detect formation or dissociation of the protein complex.

Standard solid-phase ELISA assay formats are particularly useful for identifying antagonists of the protein-protein interaction. In accordance with this embodiment, one of the binding partners (e.g. APS or CAP or CrkII or C3G or a portion thereof) is immobilized on a solid matrix, such as, for example an array of polymeric pins or a glass support. Conveniently, the immobilized binding partner is a fusion polypeptide comprising Glutathione-S-transferase (GST; e.g. a CAP-GST fusion), wherein the GST moiety facilitates immobilization of the protein to the solid phase support. The second binding partner (e.g. Cbl) in solution is brought into physical relation with the immobilized protein to form a protein complex, which complex is detected using antibodies directed against the second binding partner. The antibodies are generally labeled with fluorescent molecules or conjugated to an enzyme (e.g. horseradish peroxidase), or alternatively, a second labeled antibody can be used that binds to the first antibody. Conveniently, the second binding partner is expressed as a fusion polypeptide with a FLAG or oligo-histidine peptide tag, or other suitable immunogenic peptide, wherein antibodies against the peptide tag are used to detect the binding partner. Alternatively, oligo-HIS tagged protein complexes can be detected by their binding to nickel-NTA resin (Qiagen), or FLAG-labeled protein complexes detected by their binding to FLAG M2 Affinity Gel (Kodak). It will be apparent to the skilled person that the assay format described herein is amenable to high throughput screening of samples, such as, for example, using a microarray of bound peptides or fusion proteins.

A two-hybrid assay is described in U.S. Pat. No. 6,316,223 to Payan et al., incorporated herein by reference. The basic mechanism described by Payan et al. is similar to the yeast two hybrid system. In the two-hybrid system, the binding partners are expressed as two distinct fusion proteins in a mammalian host cell. In adapting the standard two-hybrid screen to the present purpose, a first fusion protein consists of a DNA binding domain which is fused to one of the binding partners, and a second fusion protein consists of a transcriptional activation domain fused to the other binding partner. The DNA binding domain binds to an operator sequence which controls expression of one or more reporter genes. The transcriptional activation domain is recruited to the promoter through the functional interaction between binding partners. Subsequently, the transcriptional activation domain interacts with the basal transcription machinery of the cell, thereby activating expression of the reporter gene(s), the expression of which can be determined. Candidate bioactive agents that modulate the protein-protein interaction between the binding partners are identified by their ability to modulate-transcription of the reporter gene(s) when incubated with the host cell. Antagonists will prevent or reduce reporter gene expression, while agonists will enhance reporter gene expression. In the case of small molecule modulators, these are added directly to the cell medium and reporter gene expression determined. On the other hand, peptide modulators are expressible from nucleic acid that is transfected into the host cell and reporter gene expression determined. In fact, whole peptide libraries can be screened in transfected cells.

Alternatively, reverse two hybrid screens, such as, for example, described by Vidal et al., Proc. Natl. Acad. Sci USA 93, 10315-10320, 1996, may be employed to identify antagonist molecules. Reverse hybrid screens differ from froward screens supra in so far as they employ a counter-selectable reporter gene, such as for example, CYH2 or LYS2, to select against the protein-protein interaction. Cell survival or growth is reduced or prevented in the presence of a non-toxic substrate of the counter-selectable reporter gene product, which is converted by said gene product to a toxic compound. Accordingly, cells in which the protein-protein interaction of the invention does not occur, such as in the presence of an antagonist of said interaction, survive in the presence of the substrate, because it will not be converted to the toxic product. For example, a portion/fragment of Cbl that binds to APS or CAP or CrkII or C3G is expressed as a DNA binding domain fusion, such as with the DNA binding domain of GAL4; and the portion of APS or CAP or CrkII or C3G that binds Cbl is expressed as an appropriate transcription activation domain fusion polypeptide (e.g. with the GAL4 transcription activation domain). The fusion polypeptides are expressed in yeast in operable connection with the URA3 counter-selectable reporter gene, wherein expression of URA3 requires a physical relation between the GAL4 DNA binding domain and transcriptional activation domain. This physical relation is achieved, for example, by placing reporter gene expression under the control of a promoter comprising nucleotide sequences to which GAL4 binds. Cells in which the reporter gene is expressed do not grow in the presence of uracil and 5-fluororotic acid (5-FOA), because the 5-FOA is converted to a toxic compound. Candidate peptide inhibitor(s) are expressed in libraries of such cells, wherein cells that grow in the presence of uracil and 5-FOA are retained for further analysis, such as, for example, analysis of the nucleic acid, encoding the candidate peptide inhibitor(s). Small molecule antagonists are determined by incubating the cells in the presence of the small molecules and selecting cells that grow or survive of cells in the presence of uracil and 5-FOA.

Alternatively, a protein recruitment system, such as that described in U.S. Pat. No. 5,776,689 to Karin et al., is used. In a standard protein recruitment system, a protein-protein interaction is detected in a cell by the recruitment of an effector protein, which is not a transcription factor, to a specific cell compartment. Upon translocation of the effector protein to the cell compartment, the effector protein activates a reporter molecule present in that compartment, wherein activation of the reporter molecule is detectable, for example, by cell viability, indicating the presence of a protein-protein interaction.

More specifically, the components of a protein recruitment system include a first expressible nucleic acid encoding a first fusion protein comprising the effector protein and one of the binding partners (e.g. APS or CAP or CrkII or C3G or a portion thereof), and a second expressible nucleic acid molecule encoding a second fusion protein comprising a cell compartment localization domain and the other binding partner (e.g. Cbl or a portion thereof). A cell line or cell strain in which the activity of an endogenous effector protein is defective or absent (e.g. a yeast cell or other non-mammalian cell), is also required, so that, in the absence of the protein-protein interaction, the reporter molecule is not expressed.

A complex is formed between the fusion polypeptides as a consequence of the interaction between the binding partners, thereby directing translocation of the complex to the appropriate cell compartment mediated by the cell compartment localization domain (e.g. plasma membrane localization domain, nuclear localization domain, mitochondrial membrane localization domain, and the like), where the effector protein then activates the reporter molecule. Such a protein recruitment system can be practiced in essentially any type of cell, including, for example, mammalian, avian, insect and bacterial cells, and using various effector protein/reporter molecule systems.

For example, a yeast cell based assay is performed, in which the interaction between Cbl and one or more of its binding partners results in the recruitment of a guanine nucleotide exchange factor (GEF or C3G) to the plasma membrane, wherein GEF or C3G activates a reporter molecule, such as Ras, thereby resulting in the survival of cells that otherwise would not survive under the particular cell culture conditions. Suitable cells for this purpose include, for example, Saccharomyces cerevisiae cdc25-2 cells, which grow at 36° C. only when a functional GEF is expressed therein, Petitjean et al., Genetics 124, 797-806, 1990) Translocation of the GEF to the plasma membrane is facilitated by a plasma membrane localization domain. Activation of Ras is detected, for example, by measuring cyclic AMP levels in the cells using commercially available assay kits and/or reagents. To detect antagonists of the protein-protein interaction of the present invention, duplicate incubations are carried out in the presence and absence of a test compound, or in the presence or absence of expression of a candidate antagonist peptide in the cell. Reduced survival or growth of cells in the presence of a candidate compound or candidate peptide indicates that the peptide or compound is an antagonist of the interaction between Cbl and one or more of its binding partners.

A “reverse” protein recruitment system is also contemplated, wherein modified survival or modified growth of the cells is contingent on the disruption of the protein-protein interaction by the candidate compound or candidate peptide. For example, NIH 3T3 cells that constitutively express activated Ras in the presence of GEF can be used, wherein the absence of cell transformation is indicative of disruption of the protein complex by a candidate compound or peptide. In contrast, NIH 3T3 cells that constitutively express activated Ras in the presence of GEF have a transformed phenotype (Aronheim et al., Cell. 78, 949-961, 1994)

In yet another embodiment, small molecules are tested for their ability to dissociate the protein complex of the invention, by an adaptation of plate agar diffusion assay described by Vidal and Endoh, TIBS 17, 374-381, 1999, which is incorporated herein by reference.

A further embodiment of the invention provides a method for determining a modulator of an interaction between Cbl or a portion of Cbl and a polypeptide selected from the group consisting of APS, CAP, CrkII and C3G or a portion of said polypeptide, said method comprising:
(i) determining the level of a protein complex selected from the group consisting of: (i) a complex comprising Cbl and APS; (ii) a complex comprising Cbl and CAP; (iii) a complex comprising Cbl and CrkII; (iv) a complex comprising Cbl and C3G; (v) a complex comprising Cbl and CAP and flotillin; and (vi) a complex comprising Cbl and CrkII and C3G in the absence of a candidate compound or candidate antibody; and
(ii) determining the level of said protein complex in the presence of a candidate compound or in the presence of said candidate antibody
   wherein a difference in the level of said protein complex at (i) and (ii) indicates that them candidate compound or candidate antibody is a modulator of said interaction.

This embodiment of the invention applies mutatis mutandis to the determination of protein complexes comprising a portion of any one or more of the protein binding partners (i.e. Cbl, APS, CAP, CrkII or C3G).

It will be understood by those skilled in the art that any one or more of the assay methods for antagonists as described herein above can be adapted for this purpose. This is because the level of the protein complex in the presence or absence of a candidate compound or antibody is related to antibody binding in the case of ELISAs, or to cell survival or growth, in, the case of hybrid screens or protein recruitment assays. ELISA-based assay formats are particularly suitable for this purpose, because they are readily quantifiable, by calibrating the detection system against known amounts of a protein standard to which the antibody binds. Such quantitation is well known to the skilled person.

It is to be understood that the modulators can be antagonists or inhibitors of complex formation or stability, or alternatively, agonists or promoters of complex formation and stability.

The modulators identified using the methods described herein are useful for the therapeutic or prophylactic treatment of diseases associated with associated with Cbl function, such as, for example, modifying fat deposition or lean muscle mass or the ratio of body fat to muscle or metabolic rate or aberrant glucose uptake or feeding behavior. In one embodiment, the compounds are used to treat a condition selected from the group consisting of: hyperglycemia, hyperinsulinemia, obesity, adult-onset obesity, non-insulin dependent diabetes mellitus, type II diabetes, glucose intolerance, and hypertrophy or hyperplasia of the islets of Langerhans.

In another embodiment, the compounds are used for cosmetic purposes, for example, by bodybuilders or persons wishing to modify their weight or body content of fat or muscle.

Accordingly, another aspect of the invention provides a method comprising administering an effective amount of a Cbl antagonist to an animal or human subject to inhibit or reduce the expression or activity of Cbl in the subject. Preferably, the subject is a subject in need of treatment, such as a subject suffering from a condition selected from the group consisting of: elevated glucose uptake, reduced appetite or dietary intake, hyperglycemia, hyperinsulinemia, enhanced fat deposition or obesity, adult-onset obesity, non-insulin-dependent diabetes mellitus, type II diabetes, glucose intolerance, and hypertrophy or hyperplasia of the islets of Langerhans.

In one embodiment, the invention also provides a method of treating a feeding disorder characterized by reduced dietary intake or suppressed appetite in a subject said method comprising administering to the subject an amount of a Cbl antagonist effective to enhance the appetite or dietary intake of the subject. The method of the invention is particularly suited to the treatment of anorexia or bulimia.

In a related embodiment, the invention also provides a method of treating a feeding disorder characterized by reduced dietary intake or suppressed appetite in a subject said method comprising administering to the subject an amount of a compound that reduces expression of functional Cbl effective to enhance the appetite or dietary intake of the subject.

Preferably, the compound or Cbl antagonist is a compound or antagonist identified using a screening method described herein.

To determine an appropriate dosage for treatment, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. For any Cbl inhibitor or antagonist used in the method of the invention, the therapeutically effective dose can be estimated initially from cell culture assays. A dose may be formulated in animal models (e.g. any one or more of the mouse models described supra having genetic obesity-diabetes syndromes, such as hyperglycemia, hyperinsulinemia, and obesity) to achieve a circulating plasma concentration range that includes the IC₅₀ (i.e., the concentration of the test Cbl inhibitor which achieves a half-maximal inhibition of symptoms) as determined in cell culture. Such information can be used to more accurately determine useful doses in humans. Levels in plasma maybe measured, for example, by high performance liquid chromatography.

In an alternative embodiment, the present invention provides for the use of a Cbl antagonist in the preparation of a medicament for the treatment of a condition selected from the group consisting of: elevated glucose uptake, reduced appetite or dietary intake, hyperglycemia, hyperinsulinemia, enhanced fat deposition or obesity, adult-onset obesity, non-insulin-dependent diabetes mellitus, type II diabetes, glucose intolerance, and hypertrophy or hyperplasia of the islets of Langerhans.

The various embodiments of the invention described herein for identifying compounds that are administered to animals are also suitably performed using isolated cells that have been previously derived from the animal or readily available as isolated cells, the only requirement being that the cell possesses the required Cbl phenotype to perform the assay. In one embodiment, the isolated cells are skeletal muscle cells, cardiac muscle cells, fibroblasts or fat cells (adipocytes). In lieu of using wild-type or normal animals having no Cbl deficiency, 3T3-L1 adipocytes can be used. The various embodiments apply mutatis mutandis to the use of such isolated cells.

The present invention is further described with reference to the following examples and the accompanying drawings.

### EXAMPLE 1

Methods

Animals were housed at the BTF facility of the Garvan Institute of Medical Research, Sydney, New South Wales, Australia. All procedures undertaken in these animals have been reviewed by the Ethics committee of the Garvan Institute. Animals were fed ad libitum with standard rodent chow and housed in 12 hour light/dark cycle. Body weight and food intake were monitored weekly for 12 weeks from weaning.

For determining glucose tolerance, animals were fasted overnight. Glucose (2 g/kg body weight) was administered into the intraperitoneal cavity following removal of a blood sample to test basal glucose concentration. Blood samples were taken at 15 min intervals for the following 90 min. Glucose assay was performed using the glucose oxidase method.

Fasting blood samples were also taken for measurement of circulating lipids and cytokines. Adipose depots, muscle, liver, brain were excised and weighed and frozen for subsequent biochemical analyses.

Temperature was measured using a Rectal probe (BAT-10, Physitemp) at the beginning and the end of the light cycle.

For determining glucose transport in muscle and adipose tissue, animals were fasted overnight and whole soleus muscle or gonadal white adipose tissue explants were incubated in vitro in the absence or presence of insulin in a Krebs Ringer buffer.

Glucose uptake was measured using the radiolabeled 2-deoxyglucose method.

For determining adipocyte size, the adipocytes were isolated using the collagenase method and incubated overnight in 2% osmium tetroxide at 37 C. Cells were mounted on a slide and images were acquired using bright field microscopy. Cell diameter was determine using Adobe Photoshop software.

Results

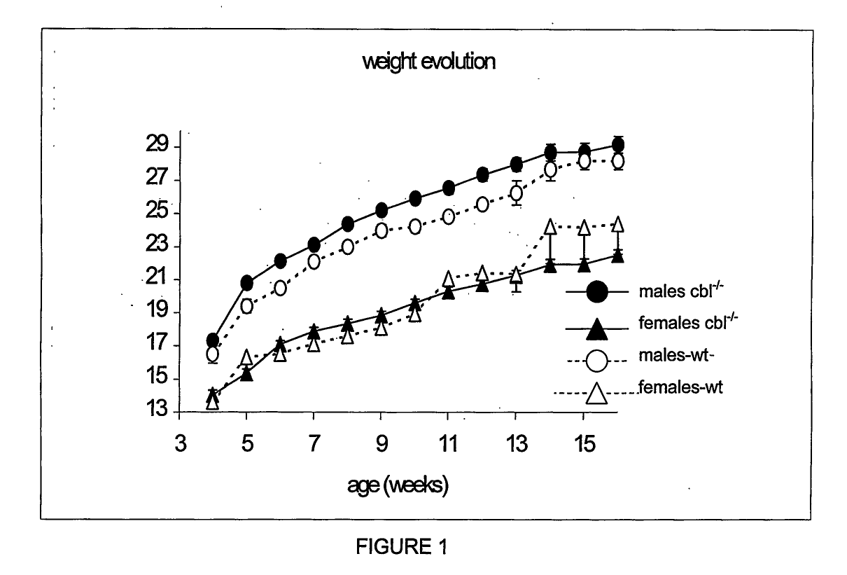

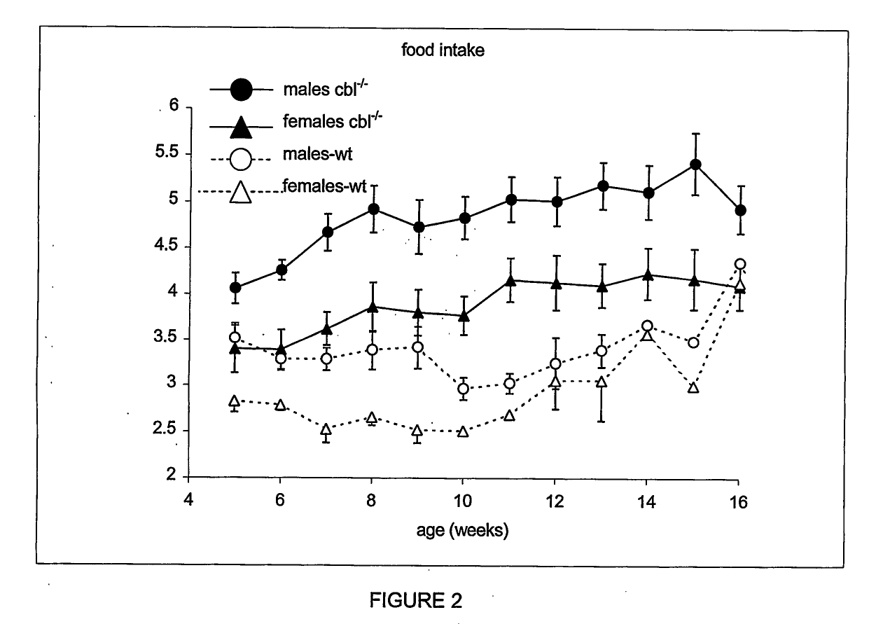

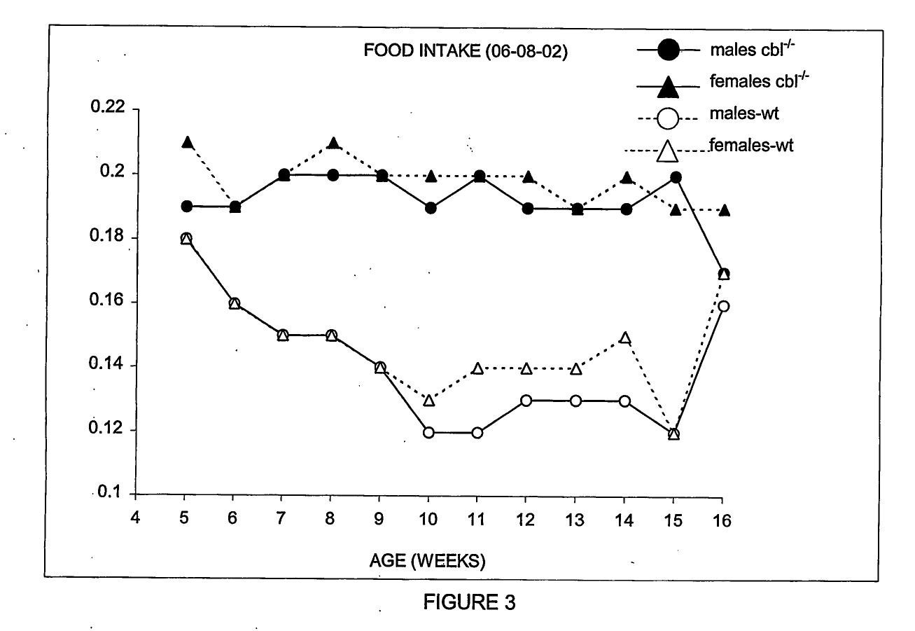

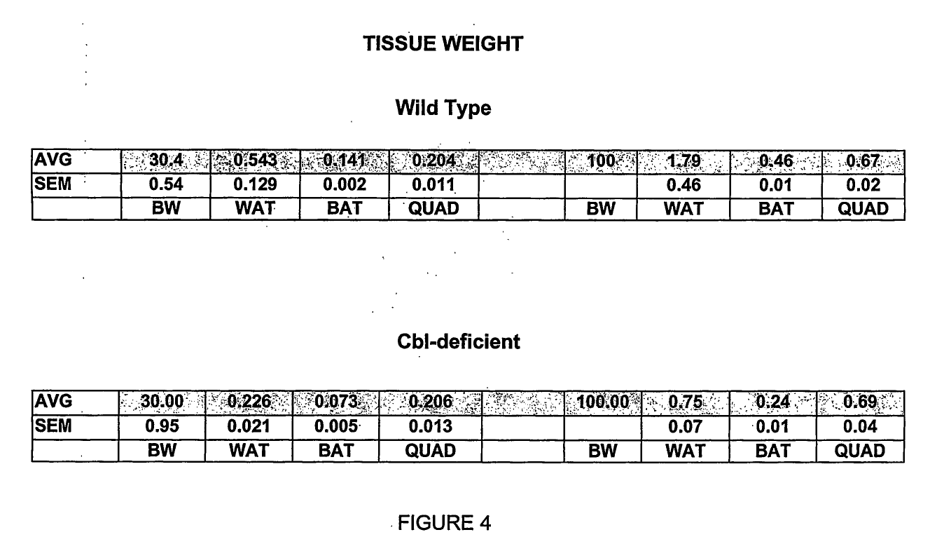

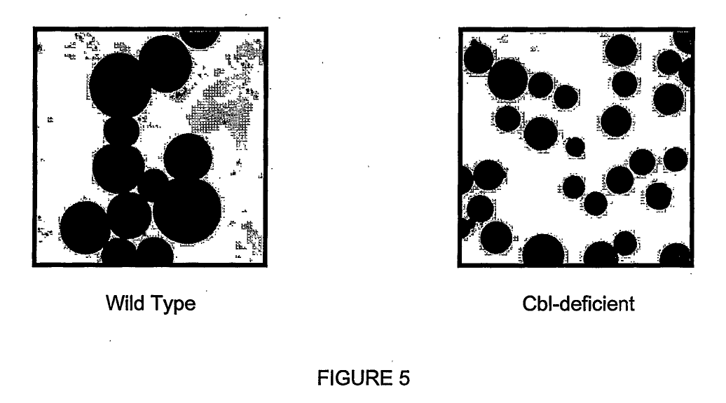

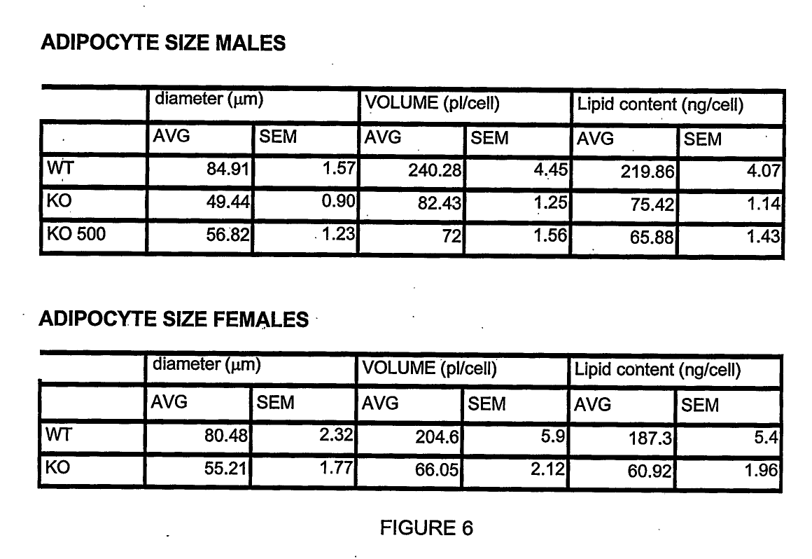

FIGS. 1 through 6 show that Cbl-deficient mice have significantly higher body weight and dietary intake, however reduced fat deposition and smaller adipocytes, compared to otherwise isogenic non-mutant animals of the same gender. The body temperature of Cbl-deficient animals is also enhanced. Surprisingly, and in contrast to what was expected, disruption of the Cbl gene lead to enhanced basal glucose uptake into both adipocytes and muscle cells. The results are also summarized in Table 1 below.

TABLE 1
Phenotype of Cbl-deficient mice

| | Phenotype in c-Cbl^{-/-} |
|---|---|
| | animals compared to wild type |
| Parameter | Cbl^{+/+} animals |
| Body weight | No change or slight increase in males |
| Food intake | Increased |
| Fat mass | Reduced |
| Adipocyte size | Reduced |
| Glucose tolerance | Increased |
| Temperature | Increased |
| Glucose transport into fat | Increased I |
| Glucose transport into muscle | Increased I |
| Fasting blood glucose | No change |

In two independent experiments, the average body temperature of Cbl-deficient males and females increased as indicated in Table 2 below:

TABLE 2
Enhanced body temperature in Cbl-deficient
animals (Average ° C. ± SEM)

| Experiment | Males | | Females | |
|---|---|---|---|---|
| No. | Cbl^{+/+} | Cbl^{-/-} | Cbl^{+/+} | Cbl^{-/-} |
| 1 | 37.11° C. ± | 38.93° C. ± | 37.60° C. ± | 38.06° C. ± |
| | 0.26° C. | 0.22° C. | 0.30° C. | 0.15° C. |
| 2 | 37.25° C. ± | 38.17° C. ± | 37.62° C. ± | 37.31° C. ± |
| | 0.16° C. | 0.14° C. | 0.09° C. | 0.12° C. |

### EXAMPLE 2

### Glucose Transport in Muscles Isolated from c-CBL^{-/-} Mice

Overnight fasted mice (16-18 weeks old) were sacrificed by cervical dislocation and the soleus muscles (SOL) and extensor digitorum longus (EDL) muscles removed immediately for incubation in vitro. After excision, SOL and EDL were transferred to individual 25 ml flasks containing 2 ml of oxygenated medium placed in a shaking water bath at 30° C. All incubation media were prepared from a pre-gassed (95% O₂/5% CO₂) stock of Krebs-Henseleit Bicarbonate buffer (KHB) (118.5 mM NaCl, 24.7 mM NaHCO_{3, 4.74} mM KCl, 1.18 mM MgSO₃, 1.18 mM KH₂PO₄ and 2.5 mM CaCl₂, pH 7.4) supplemented with 2 mM pyrovate, 8 mM mannitol and 0.1% w/v bovine serum albumin (BSA). The gas phase in the flasks was maintained at 95% O₂/5% CO₂ throughout the experiment.

The muscles were allowed to recover for 10 min. after removal of the final muscle. A 2-deoxyglucose (2-DOG) uptake assay was performed with 16 muscles from 4 mice at a time. Muscles were placed in new media without insulin, or with 300 μU/ml (2.2 nM) insulin or with 10000 μU/ml (72 nM) insulin for 30 min. An insulin concentration of 300 μU/ml was selected, as this was the insulin level found to elicit half-maximal insulin-stimulated glucose uptake in SOL in a pilot experiment. An insulin concentration of 10000 μU/ml was used to ensure maximal insulin-stimulated glucose transport.

Following the 30 min. incubation period, the medium was changed to KHB containing 2 mM pyrovate, 8 mM mannitol, 0.1% w/v BSA, 1 mM 2-DOG, [1-¹⁴C]-Mannitol and 2-[2-6 ³H]-DOG (Amersham Pharmacia Biotech Inc., Little Chafton, U.K.) to a specific activity of 0.128 μCi/ml and 0.083 μCi/ml, respectively. When present, the insulin concentration was the same as during the previous 30 min. incubation period. Labeled 2-DOG diffuses into muscle-cells through sarcolemmal glucose transporters and is trapped as 2-DOG-6-phosphate while diffusion of labeled mannitol across the plasma membrane is limited, making it suitable as an extracellular marker. After 16 min. exposure to isotopes, muscles were briefly washed in ice-cold KHB, blotted on paper, placed in Eppendorf tubes, and immediately frozen in liquid nitrogen. Muscles were stored at −80° C. until processed.

Frozen muscles were weighed and transferred to fresh Eppendorf tubes. After addition of 250 μl of 1 N NaOH the muscles were incubated at 65° C. with occasional vortexing until dissolved. Then 250 μl of 1 N HCl was added and samples were centrifuged at 17000 g for 3 min. Aliquots of 350 μl of the resultant supernatant supernatant, or an aliquot of a standard (5 μl of [1-¹⁴C]-Mannitol and 2-[2-6 ³H]-DOG in 10 ml Milli Q water), or an aliquot of media or background samples (Milli Q water) were transferred to β-scintillation vials. Then 4.5 ml of scintillation liquid (Ultima Gold XR, Perkin Elmer Life Sciences, Boston, Mass., USA) was added to each vial and, after mixing, the tubes were counted for 3 min. each in a β-scintillation counter (Beckman LS6000 SC, Beckman Coulter, Inc. Fullerton, Calif., U.S.A.).

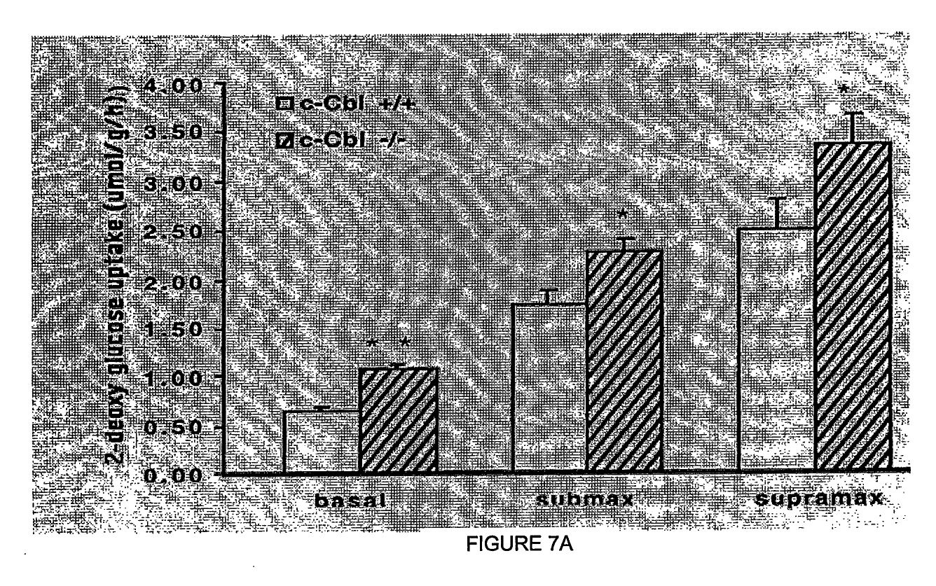

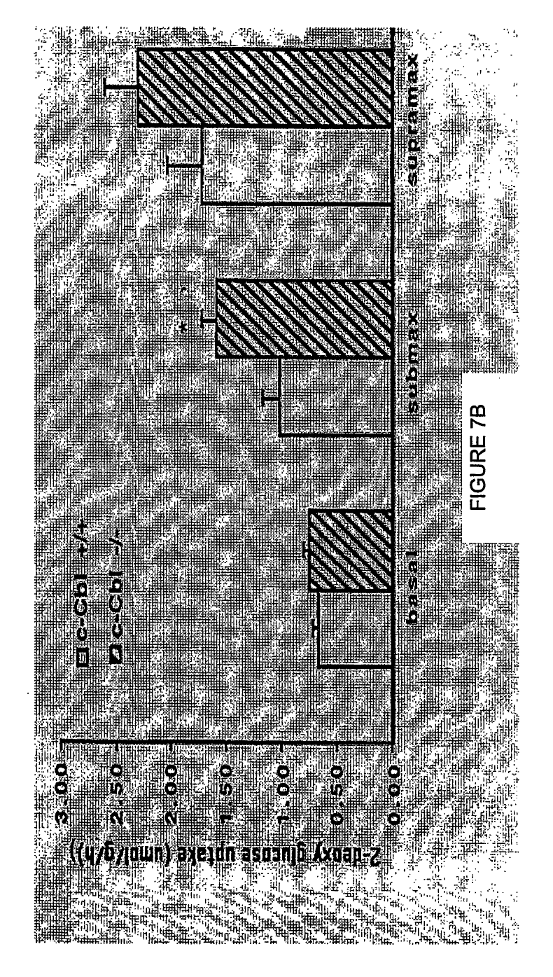

As shown in FIG. 7 the rate of 2-deoxyglucose uptake was significantly upregulated in SOL isolated from c-CBL^{-/-} mice compared to c-CBL^{+/+} controls, indicating an enhanced metabolic rate in c-CBL deficient mice. In the presence, of 300 μM/ml insulin (submax), the amount of 2-deoxyglucose uptake was significantly increased in both SOL and EDL isolated from c-CBL^{-/-} mice compared to c-CBL^{+/+} mice. When exposed to sufficient levels of insulin to ensure maximal insulin-stimulated glucose transport (ie. 1000 μU/ml insulin) there was a significant increase in 2-deoxyglucose, transport in SOL isolated from c-CBL^{-/-} mice compared to c-CBL^{+/+} mice.

These results indicate that c-CBL^{-/-} mice have increased insulin-stimulated glucose uptake (approximately 30% increase) in isolated SOL and EDL muscles, thereby indicating that c-CBL^{-/-} mice have increased peripheral insulin sensitivity.

### EXAMPLE 3

### Glucose Transport in Fat Explants from c-CBL^{-/-} Mice

Animals were sacrificed as described in Example 2 and epididymal fat pads were excised and placed in 15 ml tubes containing Hepes Krebs Ringer Phosphate Buffer (HKRP) (12.5 mM HEPES/pH 7.4, 120 mM NaCl, 6 mM KCl, 1.2 mM Mg SO₄, 1 mM CaCl₂, 0.4 mM NaH₂PO₄, 0.6 mM Na₂HPO₄) supplemented with 2 mM sodium pyruvate and 2% BSA. The tissue was then minced using scissors until pin-head size pieces were obtained. Approximately 50 μl of fat explants were placed in 24-well plate wells containing 0.45 ml of HKRP buffer. Explants were incubated in the absence or presence of 0.05 nM or 1 nM insulin for 15 minutes at 37° C. Glucose transport was assayed using the 2-deoxyglucose method essentially as described in Example 2. The assay was initiated by the addition of 100 μM 2-Deoxy-[³H] glucose (1.5 μCi/ml). Non-specific 2-Deoxy-[³H] glucose uptake was determined in the presence of Cytochalasin B (50 μM). After 10 min, the assay was terminated by washing the cells rapidly three times with ice-cold phosphate-buffered saline (PBS). Fat explants in wells were collected and weighed. Explants were then placed in vials containing scintillation liquid and counted next morning as described above. ³H cpm were normalised by tissue weight. Statistical analysis was performed using the Student's t test (Sigma Plot software).

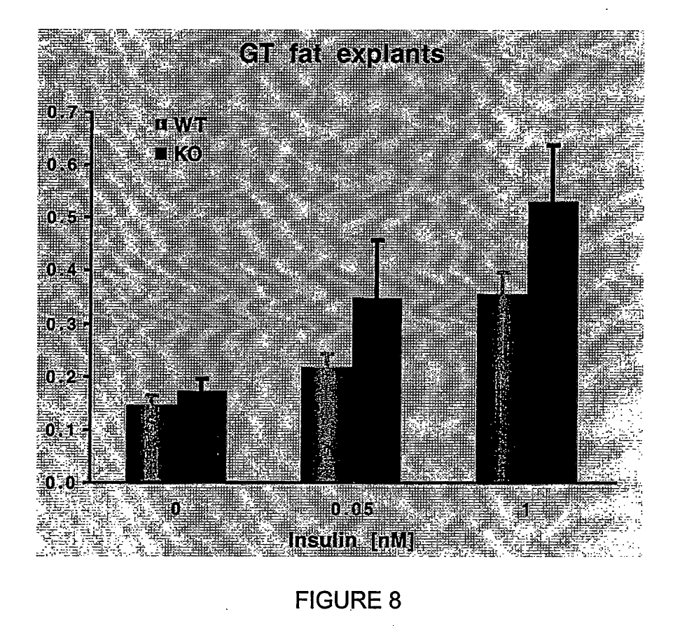

As shown in FIG. 8 the amount of 2-deoxyglucose incorporated into fat explants from c-CBL^{-/-} (KO) mice, was increased in the presence of both 0.05 nM and 0.1 nM of insulin compared to c-CBL^{+/+} (WT) mice. These data indicate that c-CBL^{-/-} mice have increased insulin-stimulated glucose uptake in fat explants compared to c-CBL^{+/+} mice, indicating increased peripheral insulin sensitivity in knockout mice.

### EXAMPLE 4

### Measurement of Resting Metabolic Rate of c-CBL^{-/-} Mice

Measurement of resting metabolic rate (i.e., muscle thermogenesis) of mice was performed essentially as described in Withers, P. C. Australian Journal of Zoology 49:445-461, 2001. Briefly, open-circuit respirometry was used to measure the rates of oxygen consumption (VO₂) and carbon dioxide production (VCO₂) at a controlled temperature of 25 (+/−1° C.).

Mice were removed from their nest box, weighed to ±0.1 g and then placed into a metabolic chamber that consisted of a 500 ml glass jar sealed with a rubber stopper. Compressed air was passed through the chamber at 400 mL min⁻¹, controlled by a Brooks 5871-A mass-flow controller, to maintain levels of O₂ above 20% and CO₂ below 1%. Excurrent air passed though a column of drierite to remove water vapour, then through a Qubit S152 infrared CO₂ analyser then a Servomex 0A 184 paramagnetic O2 analyser. At the conclusion of the trial, the mouse was removed from the chamber and its Tb was measured immediately using a plastic-sheathed thermocouple with a RS Components 611234 thermocouple meter. Baseline values of background O₂ and CO₂ were established for at least 15 min before and after each metabolic trial. Analog voltage outputs were recorded using Protek 506 (for CO₂) and Thurlby 1905a (for O₂) digital multimeters, and their RS232 outputs were recorded with a PC using a custom Visual Basic program. The metabolic system was calibrated using a butane flame (Withers 2001). Resting VO₂ and VCO₂ were determined using a custom Visual Basic program, using the formulae of Withers (2001).

As shown in Table 3 c-CBL^{-/-} mice show increased oxygen consumption compared to c-CBL^{+/+} mice, ie. c-CBL^{-/-} mice show increased VO₂ and VCO₂.

The increased oxygen consumption observed in c-Cbl^{-/-} animals indicate an elevation of resting metabolic rate. This result is consistent with lower plasma and tissue lipid levels observed in c-Cbl^{-/-} mice compared to c-Cbl^{+/+} animals (as described in Examples 2 and 3).

TABLE 3
Analysis of metabolic rate of cCBL^{-/-}mice

| | Tb | VO2 | VCO2 | | conduc- | |
|---|---|---|---|---|---|---|
| | (° C.) | (ml/g/h) | (ml/g/h) | RQ | tance | |
| WT | 36.7 | 3.5 | 2.96 | 0.85 | 0.272 | mean |
| | 0.318 | 0.120 | 0.121 | 0.05 | 0.009 | se |
| | P < | P < | P < | P < | P < | |
| | 0.02 | 0.001 | 0.002 | 0.04 | 0.01 | |
| c-CBL | 37.6 | 4.6 | 3.7 | 0.8 | 0.36 | mean |
| KO | | | | | | |
| | 0.2 | 0.22 | 0.2 | 0.022 | 0.02 | se |

### EXAMPLE 5

### Development of an In Vitro Assay for Determining Cbl E3 Ubiquitin Ligase Activity

Antibodies

Anti-ubiquitin antibody is obtained from Santa Cruz Laboratory. Anti-insulin receptor antibody 83-14 is obtained from Abcam, Inc, Cambridge Mass. 02139, USA.

Expression Vectors and Cell Lines

CHO.T-APS Cells

CHO.T cells (Ebina et al., Proc. Natl. Acad. Sci USA 82, 8014-8018, 1985) stably-expressing a Myc-tagged rat APS from the vector pIRES Hygro (Clontech) are produced as described by Ahmed et al., FEBS Letts 475, 31-34, 2000. Briefly, the Myc-tagged APS (Qian et al., Neuron 21, 1017-1029, 1998) is subcloned into pIRES Hygro and transfected into CHO.T cells using Superfect (Qiagen), and transfected cells are selected in media comprising about 800 μg/ml hygromycin. Clones are purified by limiting dilution and screened using anti-myc 9E10 monoclonal antibody prepared from a publicly available 9E10 hybridoma. Clones are maintained in media comprising about 400 μg/ml hygromycin.

CHO.T-APS Cells Expressing Human-c-Cbl and Dominant Negative c-Cbl Mutants

Several dominant-negative mutants of human c-Cbl are described in the literature, including c-Cbl G306E (SEQ ID NO: 248), c-Cbl C381A (SEQ ID NO: 250), c-Cbl Y700F (SEQ ID NO: 252), c-Cbl Y731F (SEQ ID NO: 254), c-Cbl Y774F (SEQ ID NO: 256) and c-Cbl 480 (SEQ ID NO: 260). These known mutants provide useful negative controls to assess the ability of a putative inhibitor of c-Cbl-mediated ubiquitination of IR-β in isolated cells. Accordingly, the open reading frames encoding these dominant negative mutants (SEQ ID Nos: 247, 249, 251, 253, 255 and 259, respectively) are separately sub-cloned into the retroviral expression vector pBabe-Puro retroviral expression vector (Morgenstern and Land, Nucleic Acids Res. 18, 3587-3596, 1990), to produce the control vectors pBabe-Cbl G306E, pBabe-C381A pBabe-Cbl Y700F, pBabe-Cbl Y731F, pBabe-Y774F and pBabe-Cbl 480.

A further control plasmid comprising the full-length human c-Cbl open reading frame is also produced in the pBabe vector.

A test plasmid is also produced that are capable of expressing a further putative dominant negative mutant of human c-Cbl, designated c-Cbl Y700F/Y731F/Y774F (SEQ ID NO: 258). This mutant has mutations in the open reading frame of c-Cbl that result in three tyrosine residues that would normally be phosphorylated being substituted for phenylalanine. The nucleotide sequence of the open reading frame encoding c-Cbl Y-700F/Y731F/Y774F is set forth in SEQ ID NO: 257. The mutant is produced using the Stratagene Quick Change mutagenesis kit according to the manufacturer's instructions. Confirmation of the correct mutations is obtained by standard sequence analysis. The open reading frame encoding c-Cbl Y700F/Y731F/Y774F is cloned into pBabe-Puro, to produce the test vector pBabe-Cbl Y700F/Y731F/Y774F.

BOSC23 packaging cells (Pear et al., Proc. Natl. Acad. Sci. USA, 90, 8392-8396, 1993) are maintained in DMEM containing 10% FBS at 37° C. in a humidified atmosphere of 10% CO₂. Cells are then transfected by calcium phosphate co-precipitation with 10 μg each of the plasmids.

Transiently produced viral supernatants are used to infect CHO.T-APS cells in the presence of 4 μg/ml Polybrene. After elimination of uninfected cells by puromycin, stable cell lines are maintained in DMEM containing 10% FBS.

Alternatively, CHO.T-APS cells are transfected using Superfect (Qiagen), and transfected cells are selected, purified by limiting dilution and maintained in media comprising puromycin and 400 μg/ml hygromycin.

CHO.T-APS Cells Expressing siRNAs and shRNAs Targeting Human c-Cbl Expression

The native human c-Cbl gene sequence (SEQ ID NO: 261; GenBank Accession No. X57110) was analyzed using the siRNA Target Finder software available from Ambion, Inc. (Austin, Tex., USA) to determine those sequences most likely to silence c-Cbl expression when used to produce siRNA and/or shRNA (Table 4; SEQ ID NOs: 2-239). Further sequence analysis using the BLAST programme indicated that several of these sequences were not present in the murine c-Cbl gene, in particular SEQ ID NOS: SEQ ID Nos: 57-59, 117-121, 175-177 and 235-239.

Oligonucleotides molecules are chemically synthesised that consist of the nucleotide sequences set forth in Table 4. The sense and antisense stands of the siRNA molecules set forth in are annealed and introduced directly into CHO.T-APS cells. Briefly, CHO.T-APS cells are seeded onto 96 well tissue culture plates approximately 24 hr before transfection and grown to approximately 30-70% confluence. Prior to transfection, single stranded siRNA molecules are diluted in OPTI-MEM I reduced serum medium (Ambion) to a final concentration of approximately 0.5 μM. siRNA solutions are then added to siPORT Lipid (Ambion) that has been previously diluted in OPTI-MEM I reduced serum medium. This final solution is then incubated at room temperature for approximately 20 minutes.

The CHO.T-APS cells are, then washed with OPTI-MEM I reduced serum medium and overlayed with fresh OPTI-MEM I reduced serum medium. The transfection agent/siRNA-complex is then added to each well of the plate, and plates incubated for 4 hours at 37° C. supplemented with 5% CO₂. Following 4 hours additional DMEM (supplemented with 10% FBS) is added to each well, and cells cultured at 37° C. supplemented with 5% CO₂.

For construction of shRNA, a loop sequence (TTCAAGAGA, SEQ ID NO: 246) is positioned between complementary sense and antisense strands (SEQ ID NOs: 4-239) to facilitate hairpin formation. Oligonucleotides that are complementary to the assembled shRNA are also produced synthetically. Both strands of each shRNA molecule are then mixed in equimolar amounts, heated to 95° C. for 10 minutes and allowed to cool. Double stranded shRNA-encoding DNAs are cloned into the pAdTrack-HP expression vector (Zhao et al., Gene 316: 137-141-2003), upstream of the Pol III transcriptional termination sequence. The pAdTrack-HP expression vector comprises BglII and HindIII recognition sequences to facilitate directional cloning of shRNA-upstream of the Pol III transcriptional terminator.

Control plasmids comprising the full-length human c-Cbl open reading frame in the sense and antisense orientations are also produced in the pAdTrack-HP expression vector.

Adenoviruses comprising the shRNA constructs are produced essentially as described by He et al., Proc. Natl. Acad. Sci. USA. 95: 2509-2514, 1998. Briefly, the control vectors and the library of pAdTrack-shRNA vectors are linearized with PmeI and transfected into AdEasier-1 cells or E. coli BJ5183 cells carrying the pAdEasy-1 plasmid. The pAdEasy-1 adenoviral plasmid contains all Ad5 sequences except nucleotides 1-3,533 (encompassing the E1 genes) and nucleotides 28,130-30,820 (encompassing E3). Recombinant viral genomes are linearized with PacI and transfected into 293 cells in a six-well plate using lipofectamine 2000 (Invitrogen). Eight days after transfection, the recombinant virus is collected and subjected to one round of amplification in a T-25 flask with 1.5×10⁶ 293 cells, resulting in 2 ml of viral stocks. CHO.T-APS cells in a 96-well plate, are infected with Adenovirus comprising the pADTrack-shRNA library for 2 h, washed, and incubated with medium for 4 days.

Lysate Preparation and Sandwich ELISA Assay

CHO.T-APS cells produced as described supra to express APS in the presence or absence of a dominant negative c-Cbl protein or siRNA or shRNA against human c-Cbl are stimulated with insulin at 37° C. for 10-60 mins, and lysates prepared as described by Kotani et al., Biochem J. 335, 103-109, 1998; and Ahmed et al, Biochem J., 341, 665-668, 1991. Briefly, cells are washed in ice-cold phosphate-buffered saline and then lysed for about 30 min at 4° C. with buffer containing 50 mM Tris-HCl, pH 8.0, 135 mN NaCl, 1% Triton X-100, 1.0 mM EDTA, 1.0 mM sodium pyrophosphate, 1.0 mM sodium orthovanadate, 10 mM NaF and protease inhibitors (Roche Diagnostics; 1 tablet per 7 ml buffer).

Anti-insulin receptor antibody 83-14 is diluted in coating solution (50 mM sodium carbonate, pH 9.6; or 20 mM Tris-HCl, pH 8.5; or 10 mM PBS, pH 7.2) to a concentration of about 1-10 μg/ml protein. The diluted antibody is adsorbed onto the wells of 96 or 256 well microtiter plates by adding 50-100 μl antibody in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Cell lysates are added to the wells of replica plates, optionally comprising 1-10% (w/v) BSA, and the plates are incubated at 4° C. overnight or at room temperature for 1 hour to overnight. The plates are again emptied and washed as 3-5 times as before. Anti-ubiquitin antibody at a concentration 0.1-1.0 μg/ml protein in 1× blocking solution is added to the wells and the plates are incubated for about 1 hour at room temperature. Plates are again washed as before.

To detect the anti-ubiquitin antibody bound to the plates, a tertiary antibody solution comprising goat anti-mouse Ig conjugated to horseradish peroxidase enzyme (e.g., Alpha Diagnostic International, Inc., San Antonio, Tex. 78238 USA) is employed. The tertiary antibody is diluted in 1× blocking solution at a final concentration of about 0.1-1.0 μg/ml protein. About 100 μl diluted-tertiary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of each well is determined using a plate reader. Wavelengths used will depend on the substrate employed e.g., ABTS (405-410 nm), TMB (non-stopped 620-650 nm, stopped 450 nm), OPD (non-stopped 450 nm), stopped 490 nm), pNPP (405-410 nm), BluePhosÔ (595-650 nm).

Results

Cell lysates derived from non-transfected CHO.T-APS cells or CHO.T-APS cells transfected with control vectors expressing human c-Cbl in the sense orientation will have detectable levels of insulin reduced ubiquitination of the insulin receptor, as determined by sandwich ELISA. In marked contrast, cell lysates derived from cells comprising the dominant-negative mutant constructs pBabe-Cbl G306E, pBabe-C381A pBabe-Cbl Y700F, pBabe-Cbl Y731F, pBabe-Y774F and pBabe-Cbl 480 will bind reduced or non-detectable levels of anti-ubiquitin antibody following antibody-capture of the insulin receptor. Cell lysates from cells transfected with the pBabe-C381A vector, which carries a mutation in the RING domain of c-Cbl, will have particularly low levels of ubiquitinated insulin receptor.

Such data suggest that insulin-induced CHO.T-APS cells provide a useful cellular system for assaying c-Cbl-mediated ubiquitination of the insulin receptor. Such data also suggest that the sandwich ELISA provides a useful screen for identifying agonists and antagonists of c-Cbl-mediated ubiquitination of the insulin receptor.

Proceeding on this basis, cells expressing a c-Cbl antisense RNA or the dominant negative mutant c-Cbl Y700F/Y731F/Y774F are introduced into CHO.T-APS cells and insulin-induced ubiquitination of the insulin receptor is assayed as for the control plasmids using the sandwich ELISA assays herein. Reduced levels of ubiquitin bound to the insulin, receptor relative to the level of ubiquitin bound to the receptor in lysates from cells expressing the full-length c-Cbl open reading frame in the sense orientation, indicate that these molecules are also effective antagonists of c-Cbl-mediated ubiquitination.

Additionally, an Adenovirus (Ad5) library that express a series of pADTrack-shRNAs designed, against human c-Cbl expression (Table 4) is shotgun-cloned into CHO.T-APS cells, and insulin-induced ubiquitination of the insulin receptor is determined. A control plasmid comprising the full-length open reading frame of wild-type human c-Cbl is also introduced. Empty vector controls and non-transfected controls are also employed. Those cells that produce lysates having reduced levels of ubiquitinated insulin receptor as determined by sandwich ELISA are retained for further analysis, such as for introduction into animal models for validation. Alternatively, or in addition, the corresponding shRNAs are introduced into C2C12 cells in the pBabe vector for assessment of their effects on muscle thermogenesis.

TABLE 4

Human c-Cbl siRNA oligonucleotides for silencing
human c-Cbl gene expression

| | | | |
|---|---|---|---|
| | SEQ | | SEQ |
| | ID | Antisense | ID |
| Sense strand siRNA | NO: | strand siRNA | NO: |

| | | | |
|---|---|---|---|
| CGTGAAGAAGAGCTCTGGGTT | 4 | CCCAGAGCTCTTCTTCACGTT | 122 |
| GAAGATGGTGGAGAAGTGCTT | 5 | GCACTTCTCCACCATCTTCTT | 123 |
| GATGGTGGAGAAGTGCTGGTT | 6 | CCAGCACTTCTCCACCATCTT | 124 |
| GTGCTGGAAGCTCATGGACTT | 7 | GTCCATGAGCTTCCAGCACTT | 125 |
| GCTCATGGACAAGGTGGTGTT | 8 | CACCACCTTGTCCATGAGCTT | 126 |
| GGTGGTGCGGTTGTGTCAGTT | 9 | CTGACACAACCGCACCACCTT | 127 |
| CCCAAAGCTGGCGCTAAAGTT | 10 | CTTTAGCGCCAGCTTTGGGTT | 128 |
| CCCAAAGCTGGCGCTAAAGTT | 11 | CTTTAGCGCCAGCTTTGGGTT | 129 |
| AGCTGGCGCTAAAGAATAGTT | 12 | CTATTCTTTAGCGCCAGCTTT | 130 |
| AGAATAGCCCACCTTATATTT | 13 | ATATAAGGTGGGCTATTCTTT | 131 |
| TAGCCCACCTTATATCTTATT | 14 | TAAGATATAAGGTGGGCTATT | 132 |
| GATATGAGGGGAAGATGGATT | 15 | TCCATCTTCCCCTCATATCTT | 133 |
| GATGGAGACACTTGGAGAATT | 16 | TTCTCCAAGTGTCTCCATCTT | 134 |
| CTAAGCAAACCATAAGCCTTT | 17 | AGGCTTATGGTTTGCTTAGTT | 135 |
| GCAAACCATAAGCCTCTTCTT | 18 | GAAGAGGCTTATGGTTTGCTT | 136 |
| ACCATAAGCCTCTTCAAGGTT | 19 | CCTTGAAGAGGCTTATGGTTT | 137 |
| GCCTCTTCAAGGAGGGAAATT | 20 | TTTCCCTCCTTGAAGAGGCTT | 138 |
| GAAAGAATGTATGAGGAGATT | 21 | TCTCCTCATACATTCTTTCTT | 139 |
| AGAATGTATGAGGAGAATTTT | 22 | AATTCTCCTCATACATTCTTT | 140 |
| TGTATGAGGAGAATTCTCATT | 23 | TGAGAATTCTCCTCATACATT | 141 |
| TTCTCAGCCTAGGCGAAACTT | 24 | GTTTCGCCTAGGCTGAGAATT | 142 |
| ACCTAACCAAACTGTCCCTTT | 25 | AGGGACAGTTTGGTTAGGTTT | 143 |
| CCAAACTGTCCCTCATCTTTT | 26 | AAGATGAGGGACAGTTTGGTT | 144 |
| ACTGTCCCTCATCTTCAGCTT | 27 | GCTGAAGATGAGGGACAGTTT | 145 |
| GGAATCTTTCCAAGTGGACTT | 28 | GTCCACTTGGAAAGATTCCTT | 146 |
| TCTTTCCAAGTGGACTCTTTT | 29 | AAGAGTCCACTTGGAAAGATT | 147 |
| GTGGACTCTTTCAGGGAGATT | 30 | TCTCCCTGAAAGAGTCCACTT | 148 |
| AGCAGATGCTGCGGAATTTTT | 31 | AAATTCCGCAGCATCTGCTTT | 149 |
| GACAATAGTCCCTTGGAAGTT | 32 | CTTCCAAGGGACTATTGTCTT | 150 |
| TAGTCCCTTGGAAGAGCTTTT | 33 | AAGCTCTTCCAAGGGACTATT | 151 |
| GAGCTTTCGACAGGCTCTATT | 34 | TAGAGCCTGTCGAAAGCTCTT | 152 |
| GTGCATCCCATCAGTTCTGTT | 35 | CAGAACTGATGGGATGCACTT | 153 |
| ATCCACTATTGATCTGACCTT | 36 | GGTCAGATCAATAGTGGATTT | 154 |
| TTTGACATCTTTACCCGACTT | 37 | GTCGGGTAAAGATGTCAAATT | 155 |
| TTGGAACAGCCTTGCTGTATT | 38 | TACAGCAAGGCTGTTCCAATT | 156 |
| CAGCCTTGCTGTAACTCATTT | 39 | ATGAGTTACAGCAAGGCTGTT | 157 |
| CTCATCOTGGCTACATGGCTT | 40 | GCCATGTAGCCAGGATGAGTT | 158 |
| GTGAAAGCTCGGCTCCAGATT | 41 | TCTGGAGCCGAGCTTTCACTT | 159 |
| AGCTCGGCTCCAGAAATTCTT | 42 | GAATTTCTGGAGCCGAGCTTT | 160 |
| ATTCATTCACAAACCTGGCTT | 43 | GCCAGGTTTGTGAATGAATTT | 1161 |
| ACCTGGCAGTTATATCTTCTT | 44 | GAAGATATAACTGCCAGGTTT | 162 |
| CATTCTCCAGACAATCCCTTT | 45 | AGGGATTGTCTGGAGAATGTT | 163 |
| TCCCTCACAATAAACCTCTTT | 46 | AGAGGTTTATTGTGAGGGATT | 164 |
| TAAACCTCTCTTCCAAGCATT | 47 | TGCTTGGAAGAGAGGTTTATT | 165 |
| ACCTCTCTTCCAAGCACTGTT | 48 | CAGTGCTTGGAAGAGAGGTTT | 166 |
| GCACTGATTGATGGCTTCATT | 49 | TGAAGCCATCAATCAGTGCTT | 167 |
| GGCTTCTATTTGTTTCCTGTT | 50 | CAGGAAACAAATAGAAGCCTT | 168 |
| ATCAGAATCCTGATCTGACTT | 51 | GTCAGATCAGGATTCTGATTT | 169 |
| TCCTGATCTGACTGGCTTATT | 52 | TAAGCCAGTCAGATCAGGATT | 170 |
| CCAACTCCCCAAGACCATATT | 53 | TATGGTCTTGGGGAGTTGGTT | 171 |
| CTCCCCAAGACCATATCAATT | 54 | TTGATATGGTCTTGGGGAGTT | 172 |
| GACCATATCAAAGTGACCCTT | 55 | GGGTCACTTTGATATGGTCTT | 173 |
| AGTGACCCAGGAACAATATTT | 56 | ATATTGTTCCTGGGTCACTTT | 174 |
| CAATATGAATTATACTGTGTT | 57 | CACAGTATAATTCATATTGTT | 175 |
| TATGAATTATACTGTGAGATT | 58 | TCTCACAGTATAATTCATATT | 176 |
| TTATACTGTGAGATGGGCTTT | 59 | AGCCCATCTCACAGTATAATT | 177 |
| TGATAAGGATGTAAAGATTTT | 60 | AATCTTTACATCCTTATCATT | 178 |
| GGATGTAAAGATTGAGCCCTT | 61 | GGGCTCAATCTTTACATCCTT | 179 |
| AGATTGAGCCCTGTGGACATT | 623 | TGTCCACAGGGCTCAATCTTT | 180 |
| TCAGAAGGTCAGGGCTGTCTT | 63 | GACAGCCCTGACCTTCTGATT | 181 |
| GGTCAGGGCTGTCCTTTCTTT | 64 | AGAAAGGACAGCCCTGACCTT | 182 |
| ATTAAAGGTACTGAACCCATT | 65 | TGGGTTCAGTACCTTTAATTT | 183 |
| AGGTACTGAACCCATCGTGTT | 66 | CACGATGGGTTCAGTACCTTT | 184 |
| CCCATCGTGGTAGATCCGTTT | 67 | ACGGATCTACCACGATGGGTT | 185 |
| ATTATGATGATGATGATGATT | 68 | TCATCATCATCATCATAATTT | 186 |
| CGAGCTGATGATACTCTCTTT | 69 | AGAGAGTATCATCAGCTCGTT | 187 |
| GGAATTGGCTGGTGCCAAGTT | 70 | CTTGGCACCAGCCAATTCCTT | 188 |
| TTGGCTGGTGCCAAGGTGGTT | 71 | CCACCTTGGCACCAGCCAATT | 189 |
| CGGCCGCCTTCTCCATTCTTT | 72 | AGAATGGAGAAGGCGGCCGTT | 190 |
| GTGCTTCTGCTCTTGGAACTT | 73 | GTTCCAAGAGCAGAAGCACTT | 191 |
| CTGCTTCTAAGGCTGCTTCTT | 74 | GAAGCAGCCTTAGAAGCAGTT | 192 |
| GGCTGCTTCTGGCTCCCTTTT | 75 | AAGGGAGCCAGAAGCAGCCTT | 193 |
| AGACAAACCATTGCCAGTATT | 76 | TACTGGCAATGGTTTGTCTTT | 194 |
| ACCATTGCCAGTACCTCCCTT | 77 | GGGAGGTACTGGCAATGGTTT | 195 |
| TCCCGACCTCAAAGACGCCTT | 78 | GGCGTCTTTGAGGTCGGGATT | 196 |
| AGACGCCCCTTGCCTTGTATT | 79 | TACAAGGCAAGGGGCGTCTTT | 197 |
| TCCCCAAAGTACCAGTATCTT | 80 | GATACTGGTACTTTGGGGATT | 198 |
| AGTACCAGTATCTGCCCCATT | 81 | TGGGGCAGATACTGGTACTTT | 199 |
| GTTCCAGTGATCCCTGGACTT | 82 | GTCCAGGGATCACTGGAACTT | 200 |
| GAGAATTAACCAACCGGCATT | 83 | TGCCGGTTGGTTAATTCTCTT | 201 |
| TTAACCAACCGGCACTCACTT | 84 | GTGAGTGCCGGTTGGTTAATT | 202 |
| CCAACCGGCACTCACTTCCTT | 85 | GGAAGTGAGTGCCGGTTGGTT | 203 |
| CCGGCACTCACTTCCATTTTT | 86 | AAATGGAAGTGAGTGCCGGTT | 204 |
| ATGGAGCCCAGACCAGATGTT | 87 | CATCTGGTCTGGGCTCCATTT | 205 |
| GCACGTTCAGTCTGGATACTT | 88 | GTATCCAGACTGAACGTGCTT | 206 |
| TAGCAGCCCATTAGTAGGTTT | 89 | ACCTACTAATGGGCTGCTATT | 207 |
| TCAAACCTTCCTCATCTGCTT | 90 | GCAGATGAGGAAGGTTTGATT | 208 |
| ACCTTCCTCATCTGCCAATTT | 91 | ATTGGCAGATGAGGAAGGTTT | 209 |
| TGCCATTTATTCTCTGGCTTT | 92 | AGCCAGAGAATAAATGGCATT | 210 |
| CTGCCACCTGGGGAGCAATTT | 93 | ATTGCTCCCCAGGTGGCAGTT | 211 |
| TGTGAGGGTGAAGAGGACATT | 94 | TGTCCTCTTCACCCTCACATT | 212 |
| GAGGACACAGAGTACATGATT | 95 | TCATGTACTCTGTGTCCTCTT | 213 |
| GCAATGTATAATATTCAGTTT | 96 | ACTGAATATTATACATTGCTT | 214 |
| TGTATAATATTCAGTCCCATT | 97 | TGGGACTGAATATTATACATT | 215 |
| TATTCAGTCCCAGGCGCCATT | 98 | TGGCGCCTGGGACTGAATATT | 216 |
| CACTGGTCCCGAGGAGTCATT | 99 | TGACTCCTCGGGACCAGTGTT | 217 |
| TGAGGATGATGGGTATGATTT | 100 | ATCATACCCATCATCCTCATT | 218 |
| CTCTCTCAGATATCTCTAATT | 101 | TTAGAGATATCTGAGAGAGTT | 219 |
| TGCCAGCTCCTCCTTTGGCTT | 102 | GCCAAAGGAGGAGCTGGCATT | 220 |
| CAAATGTCACTGAAGGTTCTT | 103 | GAACCTTCAGTGACATTTGTT | 221 |
| ATGTCACTGAAGGTTCCCATT | 104 | TGGGAACCTTCAGTGACATTT | 222 |
| GGTTCCCAAGTTCCCGAGATT | 105 | TCTCGGGAACTTGGGAACCTT | 223 |
| GTTCCCGAGAGGCCTCCAATT | 106 | TTGGAGGCCTCTCGGGAACTT | 224 |
| CCATTCCCGCGGAGAATCATT | 107 | TGATTCTCCGCGGGAATGGTT | 225 |
| TCAACTCTGAACGGAAAGCTT | 108 | GCTTTCCGTTCAGAGTTGATT | 226 |
| CTCTGAACGGAAAGCTGGCTT | 109 | GCCAGCTTTCCGTTCAGAGTT | 227 |
| CGGAAAGCTGGCAGCTGTCTT | 110 | GACAGCTGCCAGCTTTCCGTT | 228 |
| AGCTGGCAGCTGTCAGCAATT | 111 | TTGCTGACAGCTGCCAGCTTT | 229 |
| CCTCATGAGTCAGGGGTACTT | 112 | GTACCCCTGACTCATGAGGTT | 230 |
| AGCTTTGGTCATTGCCCAGTT | 113 | CTGGGCAATGACCAAAGCTTT | 231 |
| CAACATCGAGATGGCCAAATT | 114 | TTTGGCCATCTCGATGTTGTT | 232 |
| ACATCCTCCGGGAATTTGTTT | 115 | ACAAATTCCCGGAGGATGTTT | 233 |
| TTTGTTTCCATTTCTTCTCTT | 116 | GAGAAGAAATGGAAACAAATT | 234 |
| GTGGCACCTAGAAGGGCAGTT | 117 | CTGCCCTTCTAGGTGCCACTT | 235 |
| GGGCAGGAGTTCCTTTGGTTT | 118 | ACCAAAGGAACTCCTGCCCTT | 236 |
| GTCTTGCCCTCTCTGTGGGTT | 119 | CCCACAGAGAGGGCAAGACTT | 237 |
| GATTTCAAAGTGGTGAAATTT | 120 | ATTTCACCACTTTGAAATCTT | 238 |
| TGGAGCAGCTAGTATGTTTTT | 121 | AAACATACTAGCTGCTCCATT | 239+TZ,1/32 |

### EXAMPLE 6

### ELISA for Determining the Effect of Nucleic Acid Inhibitors on c-Cbl Expression

Antibodies

Mouse antibodies against residues 595-810 of human c-Cbl protein are obtained from Clontech. Such antibodies are cross-reactive with c-Cbl proteins from chicken, dog, mouse and rat. A mouse monoclonal antibody that is reactive against residues 695-705 of human c-Cbl protein is supplied by AG Scientific, Inc. A rabbit or goat antibody that binds to the C-terminal region of human c-Cbl and is useful for detecting full-length c-Cbl protein (Santa Cruz Biotechnology, Inc).

Expression Vectors and Cell Lines

C2C12 Cells

C2C12 cells (ATCC CRL-1772; Yaffe et al., Nature 270, 725-727, 1977; Yaffe et al., Differentiation 7, 159-166, 1977) are a murine myoblast cell line that differentiates rapidly to form contractile myotubes and to produce characteristic muscle proteins. Cultures are maintained as non-confluent cultures to prevent depletion of myoblasts. Myotube formation is enhanced when cells are grown in medium supplemented with 10% (v/v) horse serum instead of fetal bovine serum.

C2C12 Cells Expressing Human c-Cbl and Dominant Negative c-Cbl Mutants

The dominant-negative mutants of human c-Cbl designated c-Cbl G306E (SEQ ID NO: 248), c-Cbl C381A (SEQ ID NO: 250), c-Cbl Y700F (SEQ ID NO: 252), c-Cbl Y731F (SEQ ID NO: 254), and c-Cbl Y774F (SEQ ID NO: 256) expressed from the retroviral expression vector pBabe-Puro (Morgenstern and Land, Nucleic Acids Res. 18, 3587-3596, 1990), to produce the control vectors pBabe-Cbl G306E, pBabe-C381A pBabe-Cbl Y700F, pBabe-Cbl Y731F, and pBabe-Y774F, essentially as described in the preceding example.

A further control plasmid comprising the full-length human c-Cbl open reading frame is also produced in the pBabe vector.

A test plasmid is also produced that is capable of expressing a further putative dominant negative mutant, of human c-Cbl, designated c-Cbl Y700F/Y731F/Y774F (SEQ ID NO: 258), as described in the preceding example.

BOSC23 packaging cells (Pear et al. Proc. Natl. Acad. Sci. USA, 90, 8392-8396, 1993) are maintained in DMEM containing 10% FBS at 37° C. in a humidified atmosphere of 10% CO₂. Cells are then, transfected by calcium phosphate co-precipitation with 10 μg each of the plasmids supra.

Transiently produced viral supernatants are used to infect C2C12 cells in the presence of 4 μg/ml Polybrene. After elimination of uninfected cells by puromycin, stable cell lines are maintained in DMEM containing 10% FBS.

Alternatively, C2C12 cells are transfected using Superfect (Qiagen), and transfected cells are selected, purified by limiting dilution and maintained in media comprising puromycin and 400 μg/ml hygromycin.

C2C12 Cells Expressing siRNAs and shRNAs Targeting Human c-Cbl Expression

SiRNAs and shRNAs and control vectors are produced as described in the preceding example.

The siRNA oligonucleotides are introduced directly into C2C12 cells by seeding the cells onto 96 well tissue culture plates approximately 24 hr before transfection, growing the cells approximately 30-70% confluence (it is preferable to ensure that confluence is not reached to prevent myoblast depletion) and immediately prior to transfection, diluting single stranded siRNA molecules in OPTI-MEM I reduced serum medium (Ambion) to a final concentration of approximately 0.5 μM, adding the siRNA solutions to siPORT Lipid (Ambion) that has been previously diluted in OPTI-MEM I reduced serum medium and incubating the reaction mixture at room temperature for approximately 20 minutes.

The C2C12 cells are then washed with OPTI-MEM I reduced serum medium and overlayed with fresh OPTI-MEM I reduced serum medium. The transfection agent/siRNA complex is then added to each well of the plate, and plates incubated for 4 hours at 37° C. supplemented with 5% CO₂. Following 4 hours additional DMEM (supplemented with 10% FBS) is added to each well, and cells cultured at 37° C. supplemented with 5% CO₂.

For stable introduction of shRNAs into C2C12 cells, Adenovirus comprising the pADTrack-shRNA library produced as described in the preceding example is used to infect C2C12 cells in a 96-well plate for 2 h, after which time the cells are washed and incubated with medium for 4 days.

Lysate Preparation

C2C12 cells produced as described supra are lysed for about 30 min at 4° C. with buffer containing 50 mM Tris-HCl, pH 8.0, 135 mN NaCl, 1% Triton X-100, 1.0 mM EDTA, 1.0 mM sodium pyrophosphate, 1.0 mM sodium orthovanadate, 10 mM NaF and protease inhibitors (Roche Diagnostics; 1 tablet per 7 ml buffer).

ELISA Assay Format I

Cell lysates are diluted in coating solution (50 mM sodium carbonate, pH 9.6; or 20 mM Tris-HCl, pH 8.5; or 10 mM PBS, pH 7.2) to a concentration of about 0.1-10 μg/ml protein. The diluted antigen is adsorbed onto the wells of 96 or 256 well microtiter plates by adding 50-100 μl antigen in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Anti-Cbl antibodies at a concentration 0.1-1.0 μg/ml protein in blocking solution are added to the wells of replica plates, and the plates are incubated at 4° C. overnight or at room temperature for 1 hour to overnight. Different anti-Cbl antibodies can be added to each replica plate at this stage, for enhanced accuracy and validation of results. The plates are again emptied and washed as 3-5 times as before. To detect the anti-Cbl antibodies bound to the plates, a secondary antibody solution comprising an antibody that binds to the first antibody (e.g., goat anti-mouse Ig or goat anti-rabbit Ig) conjugated to horseradish peroxidase enzyme (e.g., Alpha Diagnostic International, Inc., San Antonio, Tex. 78238 USA) is employed. The secondary antibody is diluted in 1× blocking solution at a final concentration of about 0.1-1.0 μg/ml protein. About 100 μl diluted secondary antibody is added to each well, and, plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of the wells is read using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595-650 nm).

ELISA Assay Format II (Two-Site ELISA)

A mouse, rabbit or goat anti-human Cbl antibody is diluted in coating solution (50 mM sodium carbonate, pH 9.6; or 20 mM Tris-HCl, pH 8.5; or 10 mM PBS, pH 7.2) to a concentration of about 1-10 μg/ml protein. The diluted-antibody is adsorbed onto the wells of 96 or 256 well microtiter plates by adding 50-100 μl antibody in coating solution to the wells and incubating for about 1 hour at room temperature. The plates are emptied and then 300 μl blocking solution (1-10% (w/v) BSA in coating solution, or 1-10% (w/v) nonfat dry milk in coating solution, or 1-10% (w/v) casein in coating solution, or 1-10% (w/v) gelatin in coating solution) is added to the wells. The plates are incubated again at room temperature for about 1 hour. The plates are emptied and then washed 3-5 times using 300 μl wash solution (0.1 M phosphate-buffered saline or Tris-buffered saline (pH 7.4), 0.02%-0.05% (v/v) Tween 20) per wash. Cell lysates are added to the wells of replica plates, optionally comprising 1-10% (w/v) BSA, and the plates are incubated at 4° C. overnight or at room temperature for 1 hour to overnight. The plates are again emptied and washed as 3-5 times as before. A second anti-Cbl antibody that is different from the first antibody used and binds to a different part of the Cbl protein, at a concentration 0.1-1.0 μg/ml protein in 1× blocking solution, is added to the wells and the plates are incubated for about 1 hour at room temperature. Plates are again washed as before.

To detect the second anti-Cbl antibody bound to the plates, a tertiary antibody solution that binds to the secondary antibody (e.g., a goat anti-mouse Ig or goat anti-rabbit Ig) conjugated to horseradish peroxidase enzyme (e.g., Alpha Diagnostic International, Inc., San Antonio, Tex. 78238 USA) is employed. The tertiary antibody is diluted in 1× blocking solution at a final concentration of about 0.1-1.0 μg/ml protein. About 100 μl diluted tertiary antibody is added to each well, and plates are incubated for about 1 hour at room temperature. Plates are again emptied and washed, and reacted with horseradish peroxidase enzyme substrate (KPL, Gaithersburg, Mass. 20879-4174, USA). Reactions are stopped and the absorbances of the wells is read using a plate reader. Wavelengths used will depend on the substrate employed e.g., ABTS (405-410 nm), TMB (non-stopped 620-650 nm, stopped 450 nm), OPD (non-stopped 450 nm, stopped 490 nm), pNPP (405-410 nm), BluePhosÔ (595-650 nm).

Results

Cell lysates derived from non-transfected C2C12 cells transfected with control vectors expressing human c-Cbl in the sense orientation will have detectable levels of human c-Cbl, as determined by ELISA assay formats I or II. In marked contrast, cell lysates derived from cells comprising the dominant-negative mutant constructs pBabe-Cbl G306E, pBabe-C381A, pBabe-Cbl Y700F, pBabe-Cbl Y731F, or pBabe-Y774F will bind reduced or non-detectable levels of anti-Cbl antibody in assay format I, or following antibody-capture of Cbl and subsequent two-site ELISA according to assay format II.

Such data suggest that the effects of inhibitors of c-Cbl expression can be assayed directly by measuring c-Cbl protein levels in cell lysates from transfected C2C12 cells, in standard ELISA assay formats. Other immunoassay formats are also contemplated to be applicable in this respect.

Proceeding on this basis, c-Cbl protein levels are assayed in C2C12 cells expressing a c-Cbl antisense RNA or the dominant negative mutant c-Cbl Y700F/Y731F/Y774F using the sandwich ELISA assays herein. Reduced levels of c-Cbl protein detectable in C2C12 cells expressing these molecules, relative to the level of ubiquitin bound to the receptor in lysates from cells expressing the full-length c-Cbl open reading frame in the sense orientation, indicate that these molecules are also effective antagonists of c-Cbl expression.

Additionally, an Adenovirus (Ad5) library that expresses a series of pADTrack-shRNAs designed against human c-Cbl expression (Table 4) is shotgun-cloned into C2C12 cells, and the level of c-Cbl protein is determined. A control plasmid comprising the full-length open reading frame of wild-type human c-Cbl is also introduced. Empty vector controls and non-transfected controls are also employed. Those cells that produce lysates having reduced levels of c-Cbl protein as determined by ELISA assay format I and/or ELISA assay format II are retained for further analysis, such as for introduction into animal models for validation. Alternatively, or in addition, the corresponding shRNAs are introduced into C2C12 cells in the pBabe vector for assessment of their effects on muscle, thermogenesis.

### EXAMPLE 7

### C2C12 Cell-Based Assay for Muscle Thermogenesis

Expression Vectors and Cell Lines

C2C12 cells (ATCC CRL-1'772; Yaffe et al., Nature 270, 725-727, 1977; Yaffe et al., Differentiation 7, 159-166, 1977) are obtained, maintained, and modified to express human c-Cbl and dominant negative c-Cbl mutants, antisense molecules, siRNAs and shRNAs as described in the preceding example.

Proton Leak Kinetics.

Proton leak kinetics in C2C12 cells are determined essentially as described by Brand M. [(1995) in Mitochondria: A practical approach (Brown G and Cooper, C Eds) pp 39-62, Oxford Univ Press Oxford.]. Cells are incubated for 30 min with 0.2 μCi/ml [³H]-triphenylmethylphosphonium (TPMP), 0.2 μM TPMP and 1.5 μM taetraphenylboron as carriers, as well as 2.5 μg of oligomycin.

Respiration rates and membrane potential of cells are measured as follows: The starting, point for each curve (resting metabolic rate) is determined as that measured in the presence of oligomycin. Increasing concentrations of myxothiazol (0-0.072 μM) are added, to decrease the membrane potential and respiration rate. Finally, 2.5 μM myxothiazol and 2 μM carbonylcyanide p-trifluoromethoxy-phenylhydrazone are added to ascertain non-mitochondrial respiration. Non-mitochondrial, respiration is then subtracted from total respiration to obtain an estimate of mitochondrial respiration. Membrane potential is estimated as described by St Pierre et al J. Biol. Chem. 278, 26597-26603, 2003.

These measurements provide an assessment of mitochondrial respiration and the degree of mitochondrial uncoupling as assessed by proton leak kinetics.

Results

Non-transfected C2C12 cells, and C2C12 cells that are transfected with control vectors expressing human c-Cbl in the sense orientation, will provide a normal baseline of mitochondrial function and thermogenic activity, as determined by their proton leak kinetics. In marked contrast, C2C12 cells comprising the dominant-negative mutant constructs pBabe-Cbl G306E; pBabe-C381A, pBabe-Cbl Y700F, pBabe-Cbl Y731F, or pBabe-Y774F exhibit proton leak kinetics consistent with enhanced mitochondrial respiration rates compared to non-transfected controls, or cells that ectopically express Cbl protein.

Such data suggest that the effects of inhibitors of c-Cbl expression can be assayed directly by measuring proton leak kinetics of transfected C2C12 cells.

Proceeding on this basis, proton leak kinetics are determined for C2C12 cells expressing a c-Cbl antisense RNA or the dominant negative mutant c-Cbl Y700F/Y731F/Y774F. Enhanced mitochondrial respiration rates of C2C12 cells expressing these molecules, relative to the respiration rates of cells expressing the full-length c-Cbl open reading frame in the sense orientation, indicate that these molecules are also effective antagonists of c-Cbl expression.

Additionally, an Adenovirus (Ad5) library that expresses a series of pADTrack-shRNAs designed against human c-Cbl expression (Table 4) is shotgun-cloned into C2C12 cells, and the proton leak kinetics of the transfected cells is determined. A control plasmid comprising the full-length open reading frame of wild-type human c-Cbl is also introduced. Empty vector controls and non-transfected controls are also employed. Those cells that exhibit proton leak kinetics consistent with enhanced mitochondrial respiration rates are retained for further analysis, such as for introduction into animal models for validation.

### EXAMPLE 8

### Screening Assays Performed in Animal Models

Production of a Human c-Cbl Knock-In Mouse Model

A 5′-Cbl strain 129 mouse genomic fragment containing sequences upstream of the mouse c-Cbl gene (about 2-3 kbp) and including exon 1 and the 5′ part of intron 1 is generated by PCR from mouse chromosomal DNA or a chromosome 9 BAC comprising the c-Cbl gene and flanking sequences. A 3′-Cbl strain 129 mouse genomic fragment, containing the 3′ half of the final exon and 3′-flanking sequences of the mouse c-Cbl gene (about 2-3 kbp) is also derived by PCR.

Human genomic fragments, containing the 3′ part of intron 1 through to and including the final exon of the human c-Cbl gene are isolated by sequential digestion from plasmids.

To obtain gene targeting constructs, the 5′-Cbl mouse genomic fragment and the human genomic fragments are inserted into a pPN2T vector (Paszty et al., Nat. Genet. 11, 33-39, 1995) upstream of the neomycin-resistant gene. The mouse 3′-Cbl genomic fragment is inserted downstream of the neomycin-resistant gene. The arrangement of the inserts in the targeting vector are such that the 5′-Cbl and 3′-Cbl arms of mouse homology are interrupted by the human Cbl gene and the neomycin-resistant gene, and this cassette is upstream of the thymidine kinase gene in the vector. The targeting vector is linearized, purified, and redissolved in TE (10 mM Tris-HCl, pH 8, 1 mM EDTA), for electroporation.

A subclone of mouse strain 129 embryonic stem cell line, ESVJ (Go Germline, GenomeSystems, Inc.), is cultured on neomycin-resistant mouse fibroblast feeder layers and electroporated with 20 μg of the linearized targeting vector essentially as described by Koller, et al. Proc. Natl. Acad. Sci. USA 88, 10730-10734, 1991. Stable integrants are selected by positive-negative selection, using neomycin (G418-Geneticin; Invitrogen) at a final concentration of 200 μg/ml and 2 μM gancyclovir (FIAU, Moravek Biochemicals, Brea, Calif.). After 10-12 days, the colonies are transferred into 96-well plates and tested for successful targeting and lack of rearrangement of the introduced human Cbl gene, by Southern blotting using conventional procedures.

Approximately 10-15 embryonic stem-targeted cells are injected into the blastocele cavity of C57BL/6J embryos. Surviving blastocysts are transferred into the pseudopregnant CD-1 females. Animals chimeric by coat color are bred to C57BL/6J animals to determine their germ line competency. Heterozygous mutants are identified by Southern blotting of DNA isolated from the tail, and brother-sister mating is carried out to generate homozygous knock-in mutant mouse lines expressing human c-Cbl.

Expression of Dominant Negative Cbl Mutants in Cb^{+/+} Mouse and Human c-Cbl Knock-In Mouse

Because of the variation in adenoviral delivery to hindlimb muscles, it is preferred to employ an expression vector that permits quantitation of reporter gene expression e.g., the pAdTrack-CMV vector. Accordingly, the open reading frames encoding the dominant-negative mutants of human c-Cbl designated c-Cbl G306E (SEQ ID NO: 248), c-Cbl C381A (SEQ ID NO: 250), c-Cbl Y700F (SEQ ID NO: 252), c-Cbl Y731F (SEQ ID NO: 254), c-Cbl Y774F (SEQ ID NO: 256), c-Cbl 480 (SEQ ID NO: 260) and c-Cbl Y700F/Y731F/Y774F (SEQ ID NO: 258) as described in Example 5, are separately sub-cloned into the shuttle vector pAdTrack-CMV to permit their expression as a GFP fusion protein under the control of the CMV promoter. The GFP tag is not important to function of the Cbl protein moiety. The resultant plasmids are designated pAdTrack-CMV-G306E-GFP, pAdTrack-CMV-C381A-GFP, pAdTrack-CMV-Y700F-GFP, pAdTrack-CMV-Y731F-GFP, pAdTrack-CMV-Y774F-GFP, pAdTrack-CMV-Cbl 480-GFP, and pAdTrack-CMV-Y700F/Y731F/Y774F-GFP.

A further control plasmid comprising the full-length human c-Cbl open reading frame is also produced in the pAdTrack-CMV vector. This plasmid is designated pAdTrack-CMV-Cbl-GFP.

Adenoviruses comprising these constructs are produced essentially as described by He et al., Proc. Natl. Acad. Sci. USA. 95: 2509-2514, 1998. Briefly, the pAdTrack-CMV-based vectors are linearized with PmeI and transfected into AdEasier-1 cells or E. coli BJ5183 cells carrying the pAdEasy-1 plasmid. The pAdEasy-1 adenoviral plasmid contains all Ad5 sequences except nucleotides 1-3,533 (encompassing the E1 genes) and nucleotides 28,130-30,820 (encompassing E3). Recombinant viral genomes are linearized with PacI and transfected into adenoviral packaging 293 cells in a six-well plate using lipofectamine 2000 (Invitrogen). Eight days after transfection, the recombinant virus is collected and subjected to one round of amplification in a T-25 flask with 1.5×10⁶ 293 cells, resulting in 2 ml of viral stocks.

Adenoviruses expressing the dominant negative c-Cbl mutants are introduced into both wild type mice and the c-Cbl knock-in mouse supra, essentially as described by Allamand et al., Gene Ther. 7, 1385-1391, 2000. This is a robust system for the high efficiency (70-100%) transfer and expression of nucleic acid in mouse hindlimb skeletal muscle, wherein expression is maintained for >8 wk post infection, albeit being restricted to the muscle injected with the adenovirus. Briefly, mice (2-4 day old) are anesthetised via hypothermia and infectious adenovirus (2×10⁹) containing shRNAs is injected in saline percutaneously into several muscles within the hindlimb. Pups are reintroduced to mothers and studied at 4-8 weeks of age. Relevant muscles are excised at different times after injection (1, 2, 4 and 8 weeks).

Expression of siRNAs and shRNAS in Cbl^{+/+} Mouse and Human c-Cbl Knock-In Mouse

Adenoviruses expressing the siRNAs and shRNAs as described in Example 5 are introduced into both wild type mice and the c-Cbl knock-in mouse supra, and mice are treated and analysed, essentially as described in the preceding paragraph.

Effect of Dominant Negative Cbl Mutants, siRNAs and shRNAS on Phenotypes of Cbl^{-/-} Mouse and Human c-Cbl Knock-In Mouse.

The ability of inhibitory nucleic acids to mimic the phenotype of the Cbl^{-/-} mouse model is determined by their effect when administered to wild type (Cbl^{+/+} mice) and/or to mice in which the murine Cbl gene has been replaced with a human Cbl-encoding gene (i.e., the Cbl knock-in mouse). The latter murine model is particularly desirable for predicting effects on human c-Cbl in humans. Food intake, body weight, core temperature, oxygen consumption, in vivo glucose tolerance and whole body adiposity are determined for mice from one week of age.

A variety of parameters indicative of increased thermogenesis are also readily assessed in isolated hindlimb muscles using standard procedures, including mitochondrial size, number, and function, malonyl CoA levels as an index of the oxidation status of the mitochondria, intracellular triglyceride and glycogen levels.

Proton leak kinetics are determined on mitochondria isolated from the hindlimb muscles of mice treated with various nucleotide inhibitors. Mitochondria are isolated from muscle as previously described (St Pierre et al J. Biol. Chem. 278, 26597-26603, 2003). Leg muscle is removed from animals and homogenised in isolation buffer (100 mM KCl, 50 mM Tris, 2 mM EDTA, 0.5% BSA, pH 7.4 at 4 C. The homogenate is centrifuged at 2000×g for 5 min. The supernatant is centrifuged at 10,000×g for 10 min. The pellet from this spin is washed and resuspended in isolation buffer. Oxygen consumption rates are measured using Clark type electrode and the membrane potential is measured using a TPMP electrode. Rotenone (5 μM), oligomycin (1 μg/mg mitochondrial protein) and nigericin (80 ng/ml) are added at the start of the assay. TPMP will be added to 1.3 μM for calibration. Mitochondria are fed 94 mM succinate and the inhibitor malonate is then added gradually up to 1.3 mM to inhibit mitochondrial respiration and membrane potential. Finally, p-trifluoromethoxyphenylhydrazone (0.15 μM) is added to determine drift in the TPMP electrode. To determine that this effect is specific to muscle, similar studies are performed, using mitochondria isolated from other tissues.

Results

Wild-type mice, and Cbl knock-in mice transfected with adenovirus expressing full-length native human c-Cbl in the sense orientation, have normal phenotypes with respect to fat deposition, metabolism and metabolic rate in hindlimb muscles. These animals also provide a normal baseline of mitochondrial function and thermogenic activity, as determined by the proton leak kinetics of cells from isolated hindlimb muscles. In marked contrast, those animals administered with adenovirus expressing the dominant-negative mutant constructs pBabe-Cbl G306E, pBabe-C381A, pBabe-Cbl Y700F, pBabe-Cbl Y731F, or pBabe-Y774F exhibit muscle phenotypes that partially or completely mimic the lean muscle phenotype of the Cbl^{-/-} mouse as described in Examples 1-4. Additionally, proton leak kinetics of cells from isolated hindlimb muscles of these animals are consistent with enhanced mitochondrial respiration rates compared to control animals.

Such data suggest that the effects of inhibitors of c-Cbl expression can be assayed or validated by expressing the nucleic acid inhibitors in the hindlimb muscles of a suitable mouse model and determining the level of fat, glucose uptake, glucose transport into fat and/or muscle, muscle temperature, and resting metabolic rate, of hindlimb muscles from the animals. Additionally, proton leak kinetics of hindlimb muscles also provide a means of assessing efficacy of inhibitory molecules.

Proceeding on this basis, the level of fat, glucose uptake, glucose transport into fat and/or muscle, muscle temperature, and resting metabolic rate of hindlimb muscles from animals administered with adenovirus expressing c-Cbl antisense RNA or the dominant negative mutant c-Cbl Y700F/Y731F/Y774F. Proton leak kinetics are also determined for hindlimb muscle cells. Reduced fat mass and/or increased glucose transport into fat or muscle and/or increased muscle temperature and/or increased mitochondrial respiration rate of hindlimb muscles expressing these molecules, relative to the levels of these parameters for animals administered with adenovirus expressing the full-length c-Cbl open reading frame in the sense orientation, indicate that these molecules are also effective antagonists of c-Cbl expression.

Adenovirus (Ad5) expressing a series of shRNAs designed against human c-Cbl expression (Table 4) is also tested, in this animal model, to determine in vivo efficacy against the human Cbl gene. Reduced fat mass and/or increased temperature and/or increased glucose transport into fat or muscle and/or increased muscle temperature and/or increased mitochondrial respiration rate of hindlimb muscles expressing these molecules, relative to the levels of these parameters for animals administered with adenovirus expressing the full-length c-Cbl open reading frame in the sense orientation, indicate that these molecules are also effective antagonists of c-Cbl expression.

Molecules that produce one or more of these desired effects in animal models are entered into clinical trials.

### EXAMPLE 9

### Assays for Mitochondrial Function

Expression Vectors and Cell Lines

C2C12 cells (ATCC CRL-1772; Yaffe et al., Nature 270, 725-727, 1977; Yaffe et al., Differentiation 7, 159-166, 1977) are obtained, maintained, and modified to express human c-Cbl and dominant negative c-Cbl mutants, antisense molecules, siRNAs and shRNAs as described in example 6.

Electron Microscopy

To determine the effects of c-Cbl deletion on mitochondrial structure and function, C2C12 cells expressing dominant negative mutants, shRNA or siRNA are fixed with 2.5% glutaraldehyde, 1% paraformaldehyde and 0.03% picric acid in 0.1 M sodium cacodylate buffer pH 7.4, washed in 0.1 M cacodylate buffer, postfixed with 1% osmiumtetroxide/1.5% potassium ferrocyanide for 1 h, washed in water and stained with 1% uranyl acetate for 30 min followed by rehydration in alcohol. Samples are embedded in epon and 60 nm sections will be cut using an ultramicrotome. Mitochondrial volume density and cristae surface density of cells is measured as described by [Wiebel (1979) Stereological, methods: Practical methods for Biological morphometry, Academic press, London].

Levels of Mitochondrial Proteins

The levels of a range of mitochondrial proteins (cytochrome C, ATP synthase, UCP2, UCP3, PGC1 as well as a range of enzymes involved in metabolism of reactive oxygen species (ROS)) is determined readily by western blotting.

One hypothesis to explain our observations is that c-Cbl may control the production of reactive oxygen species (ROS). This is likely as tyrosine kinase GF receptors like the EGFR control the production of H₂O₂. Because in a c-Cbl null background the level of these receptors will be increased it is quite likely that the level of ROS will also be elevated. ROS has previously been shown to activate mitochondrial uncoupling proteins in a FFA-dependent manner (Echtay, Roussel, St Pierre et al Nature 2001). To test this hypothesis, the level of ROS in c-Cbl^{-/-} mice as well as in C2C12 cells depleted of c-Cbl, using the methods described by Arsenijevic et al Nature Genetics 26, 435-439, 2000.

1. A method of identifying a compound that is capable of modulating feeding behavior, fat deposition, metabolic rate, or the ratio of lean muscle mass to body fat in a subject, said method comprising performing an assay to measure a metabolism-associated phenotype that has been determined for a genetically modified non-human animal that comprises a genetic modification within an allele of its Cbl locus wherein said genetic modification reduces or prevents expression of a functional endogenous Cbl in said animal, and wherein said assay is conducted in the presence and absence of a compound to be tested, and determining the effect of the compound on the phenotype wherein a modified phenotype indicates that the compound is capable of modulating feeding behavior, fat deposition, metabolic rate, or the ratio of lean muscle mass to body fat in a subject.

2. The method according to claim 1 wherein the assay to determine a metabolism-associated phenotype measures Cbl-mediated ubiquitination of the insulin receptor in the presence and absence of the compound.

3. The method according to claim 2 wherein the assay comprises:
(a) providing a cell that is capable of effecting the c-Cbl-mediated ubiquitination of the insulin receptor;
(b) incubating the cell in the presence and absence of a compound to be tested; and
(c) determining c-Cbl-mediated ubiquitination of the insulin receptor in the presence and absence of the compound wherein a modified level of c-Cbl-mediated ubiquitination of the insulin receptor indicates that the compound is capable of modulating feeding behavior, fat deposition, metabolic rate, or the ratio of lean muscle mass to body fat in a subject.

4. The method according to claim 3 wherein the compound reduces or inhibits Cbl-mediated ubiquitination of the insulin receptor thereby indicating that said compound is capable of enhancing feeding behavior in a subject.

5. The method according to claim 3 wherein the compound reduces or inhibits Cbl-mediated ubiquitination of the insulin receptor thereby indicating that said compound is capable of reducing fat deposition in a subject.

6. The method according to claim 3 wherein the compound reduces or inhibits Cbl-mediated ubiquitination of the insulin receptor thereby indicating that said compound is capable of enhancing metabolic rate in a subject.

7. The method according to claim 3 wherein the compound reduces or inhibits Cbl-mediated ubiquitination of the insulin receptor thereby indicating that said compound is capable of enhancing the ratio of lean muscle mass to body fat in a subject.

8. The method according to claim 3 wherein the compound enhances or agonizes Cbl-mediated ubiquitination of the insulin receptor thereby indicating that said compound is capable of reducing feeding behavior and/or enhancing fat deposition in a subject and/or reducing metabolic rate in a subject and/or reducing the ratio of lean muscle mass to body fat in a subject.

9. The method according to claim 3 comprising performing an immunoassay wherein the level of c-Cbl-mediated ubiquitination of the insulin receptor is determined by contacting the insulin receptor with an antibody that binds to ubiquitin under conditions sufficient for an antigen-antibody complex to form and detecting the antibody bound to the receptor.

10. The method according to claim 9 further comprising contacting the insulin receptor with an antibody that binds to the insulin receptor under conditions sufficient for an antigen-antibody complex to form.

11. The method according to claim 3 comprising performing an immunoassay by a process comprising:
(a) providing a cell that is capable of effecting the c-Cbl-mediated ubiquitination of the insulin receptor;
(b) incubating the cell in the presence and absence of a compound to be tested;
(c) contacting an extract of the cell comprising the insulin receptor with an antibody that binds to the insulin receptor under conditions sufficient for an antigen-antibody complex to form thereby capturing the insulin receptor;
(d) contacting the captured insulin receptor with an antibody that binds to ubiquitin under conditions sufficient for an antigen-antibody complex to form; and
(e) detecting the antibody bound at (d).

12. The method according to claim 11 wherein the antibody bound at (d) is detected by contacting the antibody with a tertiary antibody that is capable of producing a detectable signal.

13. The method according to claim 1 wherein the assay to determine a metabolism-associated phenotype measures phosphorylation of a tyrosine residue on Cbl protein in the presence and absence of the compound.

14. The method according to claim 1 wherein the assay to determine a metabolism-associated phenotype measures the amount of Cbl protein in the cell in the presence and absence of the compound.

15. The method according to claim 14 wherein the assay comprises:
(a) providing a cell that is capable of expressing c-Cbl protein;
(b) incubating the cell in the presence and absence of a compound to be tested; and
(c) determining amount of c-Cbl protein in the cell in the presence and absence of the compound wherein a modified level of c-Cbl protein indicates that the compound is capable of modulating feeding behavior, fat deposition, metabolic rate, or the ratio of lean muscle mass to body fat in a subject.

16. The method according to claim 15 wherein the compound reduces or inhibits Cbl expression as determined by a reduced amount of Cbl protein in the cell thereby indicating that said compound is capable of enhancing feeding behavior in a subject.

17. The method according to claim 15 wherein the compound reduces or inhibits Cbl expression as determined by a reduced amount of Cbl protein in the cell thereby indicating that said compound is capable of reducing fat deposition in a subject.

18. The method according to claim 15 wherein the compound reduces or inhibits Cbl expression as determined by a reduced amount of Cbl protein in the cell thereby indicating that said compound is capable of enhancing metabolic rate in a subject.

19. The method according to claim 15 wherein the compound reduces or inhibits Cbl expression as determined by a reduced amount of Cbl protein in the cell thereby indicating that said compound is capable of enhancing the ratio of lean muscle mass to body fat in a subject.

20. The method according to claim 15 wherein the compound enhances or agonizes Cbl expression as determined by an increased amount of Cbl protein in the cell thereby indicating that said compound is capable of reducing feeding behavior and/or enhancing fat deposition in a subject and/or reducing metabolic rate in a subject and/or reducing the ratio of lean muscle mass to body fat in a subject.

21. The method according to claim 15 comprising performing an immunoassay wherein the amount of c-Cbl is determined by contacting the Cbl protein with an antibody that binds to Cbl under conditions sufficient for an antigen-antibody complex to form and detecting the antibody bound to the Cbl protein.

22. The method according to claim 21 wherein the antibody bound to the Cbl protein is detected by contacting the antibody with a secondary antibody that is capable of producing a detectable signal.

23. The method according to claim 15 comprising performing an immunoassay wherein the amount of c-Cbl is determined by contacting the Cbl protein with a primary and secondary antibody that each bind to Cbl under conditions sufficient for antigen-antibody complexes to form and detecting an antibody bound to the Cbl protein.

24. The method according to claim 23 wherein the antibody bound to the Cbl protein is detected by contacting the antibody with a secondary antibody that is capable of producing a detectable signal.

25. The method according to claim 23 wherein the primary and secondary antibody bind to different epitopes on the Cbl protein.

26. The method according to claim 15 comprising performing an immunoassay by a process comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein; and
(d) detecting the antibody bound at (e).

27. The method according to claim 15 comprising performing an immunoassay by a process comprising:
(a) providing a cell that is capable of expressing c-Cbl protein;
(b) incubating the cell in the presence and absence of a compound to be tested;
(c) contacting an extract of the cell comprising the Cbl protein with an antibody that binds to Cbl protein under conditions sufficient for an antigen-antibody complex to form thereby capturing the Cbl protein;
(d) contacting the captured Cbl protein with an antibody that binds to Cbl protein under conditions sufficient for an antigen-antibody complex to form, wherein said antibody binds to a different epitope on Cbl to the antibody at (c); and
(e) detecting the antibody bound at (d).

28. The method according to claim 26 wherein the antibody is detected by contacting the antibody with an antibody that is capable of producing a detectable signal.

29. The method according to claim 1 wherein the assay to determine a metabolism-associated phenotype measures Cbl-mediated fat and/or glucose metabolism in the cell in the presence and absence of the compound.

30. The method according to claim 29 wherein the assay to determine a metabolism-associated phenotype measures a phenotype in the presence and absence of the compound selected from the group consisting of fat mass, glucose transport, muscle thermogenesis, mitochondrial structure, mitochondrial function, and mitochondrial respiration rate.

31. The method according to claim 29 wherein the assay measures muscle thermogenesis in the cell in the presence and absence of the compound.

32. The method according to claim 31 wherein muscle thermogenesis in the presence and absence of the compound is determined by a process comprising determining the proton leak kinetics of a cell in the presence and absence of the compound.

33. The method according to claim 32 comprising:
(a) providing a cell of myoblast lineage capable of expressing c-Cbl protein;
(b) incubating the cell in the presence and absence of a compound to be tested; and
(c) determining the respiration rate and/or membrane potential of the cell in the presence and absence of the compound wherein a modified respiration rate and/or membrane potential indicates that the compound is capable of modulating feeding behavior, fat deposition, metabolic rate, or the ratio of lean muscle mass to body fat in a subject.

34. The method according to claim 33 wherein the compound enhances respiration rate and/or membrane potential of the cell thereby indicating that said compound is capable of enhancing feeding behavior in a subject.

35. The method according to claim 33 wherein the compound enhances respiration rate and/or membrane potential of the cell thereby indicating that said compound is capable of reducing fat deposition in a subject.

36. The method according to claim 33 wherein the compound enhances respiration rate and/or membrane potential of the cell thereby indicating that said compound is capable of enhancing metabolic rate in a subject.

37. The method according to claim 33 wherein the compound enhances respiration rate and/or membrane potential of the cell thereby indicating that said compound is capable of enhancing the ratio of lean muscle mass to body fat in a subject.

38. The method according to claim 33 wherein the compound reduces or inhibits respiration rate and/or membrane potential of the cell thereby indicating that said compound is capable of reducing feeding behavior and/or enhancing fat deposition in a subject and/or reducing metabolic rate in a subject and/or reducing the ratio of lean muscle mass to body fat in a subject.

39. The method according to claim 29 wherein the assay comprises:
(a) providing a compound to be tested to an animal subject that expresses a functional Cbl protein; and
(b) determining a metabolism-associated phenotype in a cell or tissue of the animal wherein a modified phenotype in the presence of the compound indicates that the compound is capable of modulating feeding behavior, fat deposition, metabolic rate, or the ratio of lean muscle mass to body fat in a subject.

40. The method according to claim 39 wherein the metabolism-associated phenotype is selected from the group consisting of fat mass, glucose transport, muscle thermogenesis, mitochondrial structure, mitochondrial function, and mitochondrial respiration rate.

41. The method according to claim 40 wherein the metabolism-associated phenotype is muscle thermogenesis.

42. The method according to claim 41 wherein muscle thermogenesis in the presence of the compound is determined by a process comprising determining the proton leak kinetics of a cell or tissue of the animal.

43. The method according to claim 41 wherein muscle thermogenesis is determined by measuring the respiration rate and/or membrane potential of a cell from the animal wherein a modified respiration rate and/or membrane potential indicates that the compound is capable of modulating feeding behavior, fat deposition, metabolic rate, or the ratio of lean muscle mass to body fat in a subject.

44. The method according to claim 43 wherein the compound enhances respiration rate and/or membrane potential of the cell thereby indicating that said compound is capable of enhancing feeding behavior in a subject.

45. The method according to claim 43 wherein the compound enhances respiration rate and/or membrane potential of the cell thereby indicating that said compound is capable of reducing fat deposition in a subject.

46. The method according to claim 43 wherein the compound enhances respiration rate and/or membrane potential of the cell thereby indicating that said compound is capable of enhancing metabolic rate in a subject.

47. The method according to claim 43 wherein the compound enhances respiration rate and/or membrane potential of the cell thereby indicating that said compound is capable of enhancing the ratio of lean muscle mass to body fat in a subject.

48. The method according to claim 43 wherein the compound reduces or inhibits respiration rate and/or membrane potential of the cell thereby indicating that said compound is capable of reducing feeding behavior and/or enhancing fat deposition in a subject and/or reducing metabolic rate in a subject and/or reducing the ratio of lean muscle mass to body fat in a subject.

49. The method according to claim 39 wherein the animal subject is a non-human animal subject.

50. The method according to claim 49 wherein the non-human animal subject expresses an endogenous native Cbl protein.

51. The method according to claim 49 wherein the non-human animal subject expresses an introduced human Cbl protein.

52. The method according to claim 49 wherein the non-human animal is a mammal.

53. The method according to claim 52 wherein the mammal is selected from the group consisting of rodent, dog, pig, bovine, sheep, horse and goat.

54. The method according to claim 53 wherein the rodent is selected from the group consisting of rabbit, rat, guinea pig and mouse.

55. The method according to claim 54 wherein the rodent is a mouse.

56. The method according to claim 39 wherein the compound is administered to muscle tissue of the animal subject.

57. The method according to claim 56 wherein the metabolism-associated phenotype is determined in muscle tissue of the animal subject.

58. The method according to claim 1 comprising comparing the effect of the compound on the metabolism-associated phenotype to a metabolism-associated phenotype for a genetically modified non-human animal that comprises a genetic modification within an allele of its Cbl locus wherein said genetic modification reduces or prevents expression of a functional endogenous Cbl in said animal, and wherein the ability of the compound to mimic or reproduce the phenotype of the genetically modified non-human animal indicates that the compound is capable of enhancing feeding behavior and/or reducing fat deposition and/or enhancing metabolic rate and/or enhancing the ratio of lean muscle mass to body fat in a subject.

59. The method according to claim 1 comprising comparing the effect of the compound on the metabolism-associated phenotype to a metabolism-associated phenotype for a non-human animal that expresses a functional Cbl protein, and wherein the ability of the compound to mimic or reproduce the phenotype of the non-human animal indicates that the compound is capable of reducing feeding behavior and/or enhancing fat deposition and/or reducing metabolic rate and/or reducing the ratio of lean muscle mass to body fat in a subject.

60. A method of identifying a compound that suppresses or reduces feeding behavior, said method comprising:
(a) administering a compound to a genetically modified non-human animal comprising a genetic modification within an allele of its Cbl locus wherein said genetic modification reduces or prevents expression of a functional endogenous Cbl in said animal; and
(b) determining the feeding behavior of the animal, wherein reduced appetite or dietary intake of the animal compared to the appetite or dietary intake of a Cbl-deficient animal to which the compound has not been administered indicates that the compound suppresses or reduces feeding behavior.

61. A method for identifying a compound that suppresses or reduces feeding behavior, said method comprising determining the ubiquitin ligase activity of a Cbl protein in the presence and absence of the compound wherein enhanced ubiquitin ligase activity of the Cbl protein in the presence of the compound indicates that the compound suppresses or reduces feeding behavior.

62. A method for identifying a compound that suppresses or reduces feeding behavior comprising determining the level of tyrosine phosphorylation of a Cbl protein in the presence and absence of the compound wherein enhanced phosphorylation of tyrosine residues in the Cbl protein in the presence of the compound indicates that the compound suppresses or reduces feeding behavior.

63. A method of identifying a compound that enhances feeding behavior said method comprising:
(a) administering a compound that suppresses appetite or dietary intake to a genetically modified non-human animal comprising a genetic modification within an allele of its Cbl locus wherein said genetic modification reduces or prevents expression of a functional endogenous Cbl in said animal and determining the feeding behavior of the animal;
(b) administering a compound to the animal and determining the feeding behavior of the animal, wherein enhanced appetite or dietary intake at (b) compared to (a) indicates that the compound enhances feeding behavior.

64. A method of identifying a compound that enhances feeding behavior comprising determining the ubiquitin ligase activity of a Cbl protein in the presence and absence of the compound wherein reduced ubiquitin ligase activity of the Cbl protein in the presence of the compound indicates that the compound enhances feeding behavior.

65. A method for identifying a compound that enhances feeding behavior comprising determining the level of tyrosine phosphorylation of a Cbl protein in the presence and absence of the compound wherein reduced phosphorylation of tyrosine residues in the Cbl protein in the presence of the compound indicates that the compound enhances feeding behavior.

66. A method of identifying a compound that modulates feeding behavior, said method comprising:
(a) administering a compound to a non-human animal expressing a functional Cbl protein and determining the feeding behavior of the animal;
(b) determining the feeding behavior of a genetically modified non-human animal comprising a genetic modification within an allele of its Cbl locus wherein said genetic modification reduces or prevents expression of a functional endogenous Cbl in said animal; and
(c) comparing the feeding behavior of the animals at (a) and (b) wherein a comparable feeding behavior between (a) and (b) indicates that the compound modulates feeding behavior.

67. A method of identifying a compound that modulates feeding behavior comprising determining the ubiquitin ligase activity of a Cbl protein in the presence and absence of the compound wherein modified ubiquitin ligase activity of the Cbl protein in the presence of the compound indicates that the compound enhances feeding behavior.

68. A method for identifying a compound that modulates feeding behavior comprising determining the level of tyrosine phosphorylation of a Cbl protein in the presence and absence of the compound wherein modified phosphorylation of tyrosine residues in the Cbl protein in the presence of the compound indicates that the compound modulates feeding behavior.

69. A method of identifying a compound that enhances fat deposition or reduces lean muscle mass or enhances the ratio of body fat to muscle or reduces metabolic rate, said method comprising:
(a) administering a compound to a genetically modified non-human animal comprising a genetic modification within an allele of its Cbl locus wherein said genetic modification reduces or prevents expression of a functional endogenous Cbl in said animal; and
(b) determining the fat content of the animal, wherein enhanced fat content of the animal compared to the fat content of a Cbl-deficient animal to which the compound has not been administered indicates that the compound enhances fat deposition or reduces lean muscle mass or enhances the ratio of body fat to muscle or reduces metabolic rate.

70. A method of identifying a compound that enhances fat deposition or reduces lean muscle mass or enhances the ratio of body fat to muscle or reduces metabolic rate comprising determining the ubiquitin ligase activity of a Cbl protein in the presence and absence of the compound wherein enhanced ubiquitin ligase activity of the Cbl protein in the presence of the compound indicates that the compound enhances fat deposition or reduces lean muscle mass or enhances the ratio of body fat to muscle or reduces metabolic rate.

71. A method for identifying a compound that enhances fat deposition or reduces lean muscle mass or enhances the ratio of body fat to muscle or reduces metabolic rate comprising determining the level of tyrosine phosphorylation of a Cbl protein in the presence and absence of the compound wherein enhanced phosphorylation of tyrosine residues in the Cbl protein in the presence of the compound indicates that the compound enhances fat deposition or reduces lean muscle mass or enhances the ratio of body fat to muscle or reduces metabolic rate.

72. A method of identifying a compound that reduces fat deposition or enhances lean muscle mass or reduces the ratio of body fat to muscle or enhances metabolic rate, said method comprising:
(a) administering a compound that enhances fat deposition or glucose uptake to a genetically modified non-human animal comprising a genetic modification within an allele of its Cbl locus wherein said genetic modification reduces or prevents expression of a functional endogenous Cbl in said animal and determining the fat content of the animal; and
(b) administering a compound to the animal and determining the fat content of the animal, wherein a similar or reduced fat content at (b) compared to (a) indicates that the compound reduces fat deposition or enhances lean muscle mass or reduces the ratio of body fat to muscle or enhances metabolic rate.

73. A method of identifying a compound that reduces fat deposition or enhances lean muscle mass or reduces the ratio of body fat to muscle or enhances metabolic rate comprising:
(a) administering a compound to a non-human animal expressing a functional Cbl protein and determining the fat content of the animal;
(b) determining the fat content of a genetically modified non-human animal comprising a genetic modification within an allele of its Cbl locus wherein said genetic modification reduces or prevents expression of a functional endogenous Cbl in said animal; and
(c) comparing the fat contents of the animals at (a) and (b) wherein a comparable fat content between (a) and (b) indicates that the compound reduces fat deposition or enhances lean muscle mass or reduces the ratio of body fat to muscle or enhances metabolic rate.

74. A method of identifying a compound that reduces fat deposition or enhances lean muscle mass or reduces the ratio of body fat to muscle or enhances metabolic rate comprising determining the ubiquitin ligase activity of a Cbl protein in the presence and absence of the compound wherein reduced ubiquitin ligase activity of the Cbl protein in the presence of the compound indicates that the compound reduces fat deposition or enhances lean muscle mass or reduces the ratio of body fat to muscle or enhances metabolic rate.

75. A method for identifying a compound that reduces fat deposition or enhances lean muscle mass or reduces the ratio of body fat to muscle or enhances metabolic rate comprising determining the level of tyrosine phosphorylation of a Cbl protein in the presence and absence of the compound wherein reduced phosphorylation of tyrosine residues in the Cbl protein in the presence of the compound indicates that the compound reduces fat deposition or enhances lean muscle mass or reduces the ratio of body fat to muscle or enhances metabolic rate.

76. A method of identifying a compound that enhances glucose uptake, said method comprising:
(a) administering a compound to a genetically modified non-human animal comprising a genetic modification within an allele of its Cbl locus wherein said genetic modification reduces or prevents expression of a functional endogenous Cbl in said animal; and
(b) determining the glucose uptake into liver, fat or muscle cells of the animal, wherein enhanced uptake compared to the glucose uptake into liver, fat or muscle cells of a Cbl-deficient animal to which the compound has not been administered indicates that the compound enhances glucose uptake.

77. A method of identifying a compound that enhances glucose uptake comprising determining the ubiquitin ligase activity of a Cbl protein in the presence and absence of the compound wherein reduced ubiquitin ligase activity of the Cbl protein in the presence of the compound indicates that the compound reduces fat deposition or enhances glucose uptake.

78. A method for identifying a compound that enhances glucose uptake comprising determining the level of tyrosine phosphorylation of a Cbl protein in the presence and absence of the compound wherein reduced phosphorylation of tyrosine residues in the Cbl protein in the presence of the compound indicates that the compound enhances glucose uptake.

79. A method of identifying a compound that reduces glucose uptake into liver, fat or muscle cells, said method comprising:
(a) administering a compound that enhances glucose uptake to a genetically modified non-human animal comprising a genetic modification within an allele of its Cbl locus wherein said genetic modification reduces or prevents expression of a functional endogenous Cbl in said animal and determining the glucose uptake into liver, fat or muscle cells;
(b) administering a compound to the animal and determining the glucose uptake into liver, fat or muscle cells of the animal, wherein a similar or reduced uptake at (b) compared to (a) indicates that the compound reduces glucose uptake into liver, fat or muscle cells.

80. A method of identifying a compound that reduces glucose uptake into liver, fat or muscle cells comprising:
(a) administering a compound to a non-human animal expressing a functional Cbl protein and determining the glucose uptake into liver, fat or muscle cells of the animal;
(b) determining the glucose uptake into liver, fat or muscle cells of a genetically modified non-human animal comprising a genetic modification within an allele of its Cbl locus wherein said genetic modification reduces or prevents expression of a functional endogenous Cbl in said animal; and
(c) comparing the glucose uptake into liver, fat or muscle cells of the animals at (a) and (b) wherein a comparable uptake between (a) and (b) indicates that the compound reduces glucose uptake into liver, fat or muscle cells.

81. A method of identifying a compound that reduces glucose uptake comprising determining the ubiquitin ligase activity of a Cbl protein in the presence and absence of the compound wherein enhanced ubiquitin ligase activity of the Cbl protein in the presence of the compound indicates that the compound reduces glucose uptake.

82. A method for identifying a compound that reduces glucose uptake comprising determining the level of tyrosine phosphorylation of a Cbl protein in the presence and absence of the compound wherein enhanced phosphorylation of tyrosine residues in the Cbl protein in the presence of the compound indicates that the compound reduces glucose uptake.

83. The method according to claim 1 wherein the compound being tested is a protein.

84. The method according to claim 83 wherein the protein is a dominant negative mutant of Cbl.

85. The method according to claim 84 wherein the dominant negative mutant of Cbl comprises an amino acid sequence selected from the group consisting of SEQ ID NO: 248, SEQ ID NO: 250, SEQ ID NO: 252, SEQ ID NO: 254, SEQ ID NO: 256, SEQ ID NO: 258 and SEQ ID NO: 260.

102. The method according to claim 1 wherein the compound being tested is nucleic acid.

103. The method according to claim 84 wherein the nucleic acid comprises an antisense molecule, ribozyme, siRNA, or shRNA.

86. The method according to claim 85 wherein the nucleic acid comprises siRNA, or shRNA that comprises a nucleotide sequence set forth in any one of SEQ ID NOS: 4-239.

87. The method according to claim 1 wherein the compound tested is a small organic molecule.

88. The method according to claim 1 wherein the compound in an antibody that binds to a Cbl protein.

89. The method according to claim 1 further comprising formulating the identified compound for administration to a non-human animal or a human.

90. The method according to claim 1 further comprising producing or synthesizing the compound.

91. A process for identifying or determining a compound that modulates feeding behavior, fat deposition, metabolic rate, or the ratio of lean muscle mass to body fat in a subject, said process comprising:
(a) performing the method according to claim 1;
(b) optionally, determining the structure of the compound; and
(c) providing the compound or the name or structure of the compound.

92. The process according to claim 91 wherein the compound name or structure is provided in a paper form, machine-readable form, or computer-readable form.

93. The process according to claim 90 wherein the compound structure is known.

94. The process according to claim 90 wherein the compound or the name or structure of the compound is provided with an indication as to its use.

95. A process for producing a compound that modulates feeding behavior, fat deposition, metabolic rate, or the ratio of lean muscle mass to body fat in a subject, said process comprising:
(a) performing the method according to claim 1 to thereby identify or determine a compound;
(b) optionally, determining the structure of the compound or modulator;
(c) optionally, providing the name or structure of the compound or modulator; and
(d) producing the compound or modulator.

96. The process according to claim 95 wherein the compound name or structure is provided in a paper form, machine-readable form, or computer-readable form.

97. The process according to claim 95 wherein the compound or the name or structure of the compound is provided with an indication as to its use.

98. The process according to claim 97 further comprising formulating the compound in a suitable diluent or excipient.

99.-101. (canceled)


SEQUENCE LISTING (261 sequences)

SEQ ID NO: 1 - protein, 153 aa, artificial
  disrupted mouse Cbl protein
MAGNVKKSSGAGGGGSGGSGAGGLIGLMKDAFQPHHHHHHLSPHPPCTVDKKMVEKCWKLMDKVVRLCQNPNVALKNSPPYILDLLPDTYQHLRTVLSRYEGKMETLGENEYFRVFMENLMKKTKQTISLFKEGKERMYEENSQPRRNLTKLS

SEQ ID NO: 2 - protein, 896 aa, Mus musculus
MAGNVKKSSGAGGGGSGGSGAGGLIGLMKDAFQPHHHHHHLSPHPPCTVDKKMVEKCWKLMDKVVRLCQNPNVALKNSPPYILDLLPDTYQHLRTVLSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQICAENDKDVKIEPCGHLMCTSCLTSWQESEGQGCPFCRCEIKGTEPIVVDPFDPRGSGSLLRQGAEGAPSPNYDDDDDERADDSLFMMKELAGAKVERPSSPFSMAPQASLPPVPPRLDLLQQRAPVPASTSVLGTASKAASGSLHKDKPLPIPPTLRDLPPPPPPDRPYSVGAETRPQRRPLPCTPGDCPSRDKLPPVPSSRPGDSWLSRTIPKVPVATPNPGDPWNGRELTNRHSLPFSLPSQMEPRADVPRLGSTFSLDTSMTMNSSPVAGPESEHPKIKPSSSANAIYSLAARPLPMPKLPPGEQGESEEDTEYMTPTSRPVGVQKPEPKRPLEATQSSRACDCDQQIDSCTYEAMYTIQSQALSVAENSASGEGNLATAHTSTGPEESENEDDGYDVPKPPVPAVLARRTLSDISNASSSFGWLSLDGDPTNFNEGSQVPERPPKPFPRRINSERKASSYQQGGGATANPVATAPSPQLSSEIERLMSQGYSYQDIQKALVIAHNNIEMAKNILREFVSISSPAHVAT

SEQ ID NO: 3 - protein, 906 aa, Homo sapiens
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 4 - DNA, 21 bp, artificial
  siRNA sense strand oligonucleotide
cgtgaagaagagctctgggtt

SEQ ID NO: 5 - DNA, 21 bp, artificial
  siRNA sense strand oligonucleotide
gaagatggtggagaagtgctt

SEQ ID NO: 6 - DNA, 21 bp, artificial
  siRNA sense strand oligonucleotide
gatggtggagaagtgctggtt

SEQ ID NO: 7 - DNA, 21 bp, artificial
  siRNA sense strand oligonucleotide
gtgctggaagctcatggactt

SEQ ID NO: 8 - DNA, 21 bp, artificial
  siRNA sense strand oligonucleotide
gctcatggacaaggtggtgtt

SEQ ID NO: 9 - DNA, 21 bp, artificial
  siRNA sense strand oligonucleotide
ggtggtgcggttgtgtcagtt

SEQ ID NO: 10 - DNA, 21 bp, artificial
  siRNA sense strand oligonucleotide
cccaaagctggcgctaaagtt

SEQ ID NO: 11 - DNA, 21 bp, artificial
  siRNA sense strand oligonucleotide
cccaaagctggcgctaaagtt

SEQ ID NO: 12 - DNA, 21 bp, artificial
  siRNA sense strand oligonucleotide
agctggcgctaaagaatagtt

SEQ ID NO: 13 - DNA, 21 bp, artificial
  siRNA sense strand oligonucleotide
agaatagcccaccttatattt

SEQ ID NO: 14 - DNA, 21 bp, artificial
  siRNA sense strand oligonucleotide
tagcccaccttatatcttatt

SEQ ID NO: 15 - DNA, 21 bp, artificial
  siRNA sense strand oligonucleotide
gatatgaggggaagatggatt

SEQ ID NO: 16 - DNA, 21 bp, artificial
  siRNA sense strand oligonucleotide
gatggagacacttggagaatt

SEQ ID NO: 17 - DNA, 21 bp, artificial
  siRNA sense strand oligonucleotide
ctaagcaaaccataagccttt

SEQ ID NO: 18 - DNA, 21 bp, artificial
  siRNA sense strand oligonucleotide
gcaaaccataagcctcttctt

SEQ ID NO: 19 - DNA, 21 bp, artificial
  siRNA sense strand oligonucleotide
accataagcctcttcaaggtt

SEQ ID NO: 20 - DNA, 21 bp, artificial
  siRNA sense strand oligonucleotide
gcctcttcaaggagggaaatt

SEQ ID NO: 21 - DNA, 21 bp, artificial
  siRNA sense strand oligonucleotide
gaaagaatgtatgaggagatt

SEQ ID NO: 22 - DNA, 21 bp, artificial
  siRNA sense strand oligonucleotide
agaatgtatgaggagaatttt

SEQ ID NO: 23 - DNA, 21 bp, artificial
  siRNA sense strand oligonucleotide
tgtatgaggagaattctcatt

SEQ ID NO: 24 - DNA, 21 bp, artificial
  siRNA sense strand oligonucleotide
ttctcagcctaggcgaaactt

SEQ ID NO: 25 - DNA, 21 bp, artificial
  siRNA sense strand oligonucleotide
acctaaccaaactgtcccttt

SEQ ID NO: 26 - DNA, 21 bp, artificial
  siRNA sense strand oligonucleotide
ccaaactgtccctcatctttt

SEQ ID NO: 27 - DNA, 21 bp, artificial
  siRNA sense strand oligonucleotide
actgtccctcatcttcagctt

SEQ ID NO: 28 - DNA, 21 bp, artificial
  siRNA sense strand oligonucleotide
ggaatctttccaagtggactt

SEQ ID NO: 29 - DNA, 21 bp, artificial
  siRNA sense strand oligonucleotide
tctttccaagtggactctttt

SEQ ID NO: 30 - DNA, 21 bp, artificial
  siRNA sense strand oligonucleotide
gtggactctttcagggagatt

SEQ ID NO: 31 - DNA, 21 bp, artificial
  siRNA sense strand oligonucleotide
agcagatgctgcggaattttt

SEQ ID NO: 32 - DNA, 21 bp, artificial
  siRNA sense strand oligonucleotide
gacaatagtcccttggaagtt

SEQ ID NO: 33 - DNA, 21 bp, artificial
  siRNA sense strand oligonucleotide
tagtcccttggaagagctttt

SEQ ID NO: 34 - DNA, 21 bp, artificial
  siRNA sense strand oligonucleotide
gagctttcgacaggctctatt

SEQ ID NO: 35 - DNA, 21 bp, artificial
  siRNA sense strand oligonucleotide
gtgcatcccatcagttctgtt

SEQ ID NO: 36 - DNA, 21 bp, artificial
  siRNA sense strand oligonucleotide
atccactattgatctgacctt

SEQ ID NO: 37 - DNA, 21 bp, artificial
  siRNA sense strand oligonucleotide
tttgacatctttacccgactt

SEQ ID NO: 38 - DNA, 21 bp, artificial
  siRNA sense strand oligonucleotide
ttggaacagccttgctgtatt

SEQ ID NO: 39 - DNA, 21 bp, artificial
  siRNA sense strand oligonucleotide
cagccttgctgtaactcattt

SEQ ID NO: 40 - DNA, 21 bp, artificial
  siRNA sense strand oligonucleotide
ctcatcctggctacatggctt

SEQ ID NO: 41 - DNA, 21 bp, artificial
  siRNA sense strand oligonucleotide
gtgaaagctcggctccagatt

SEQ ID NO: 42 - DNA, 21 bp, artificial
  siRNA sense strand oligonucleotide
agctcggctccagaaattctt

SEQ ID NO: 43 - DNA, 21 bp, artificial
  siRNA sense strand oligonucleotide
attcattcacaaacctggctt

SEQ ID NO: 44 - DNA, 21 bp, artificial
  siRNA sense strand oligonucleotide
acctggcagttatatcttctt

SEQ ID NO: 45 - DNA, 21 bp, artificial
  siRNA sense strand oligonucleotide
cattctccagacaatcccttt

SEQ ID NO: 46 - DNA, 21 bp, artificial
  siRNA sense strand oligonucleotide
tccctcacaataaacctcttt

SEQ ID NO: 47 - DNA, 21 bp, artificial
  siRNA sense strand oligonucleotide
taaacctctcttccaagcatt

SEQ ID NO: 48 - DNA, 21 bp, artificial
  siRNA sense strand oligonucleotide
acctctcttccaagcactgtt

SEQ ID NO: 49 - DNA, 21 bp, artificial
  siRNA sense strand oligonucleotide
gcactgattgatggcttcatt

SEQ ID NO: 50 - DNA, 21 bp, artificial
  siRNA sense strand oligonucleotide
ggcttctatttgtttcctgtt

SEQ ID NO: 51 - DNA, 21 bp, artificial
  siRNA sense strand oligonucleotide
atcagaatcctgatctgactt

SEQ ID NO: 52 - DNA, 21 bp, artificial
  siRNA sense strand oligonucleotide
tcctgatctgactggcttatt

SEQ ID NO: 53 - DNA, 21 bp, artificial
  siRNA sense strand oligonucleotide
ccaactccccaagaccatatt

SEQ ID NO: 54 - DNA, 21 bp, artificial
  siRNA sense strand oligonucleotide
ctccccaagaccatatcaatt

SEQ ID NO: 55 - DNA, 21 bp, artificial
  siRNA sense strand oligonucleotide
gaccatatcaaagtgaccctt

SEQ ID NO: 56 - DNA, 21 bp, artificial
  siRNA sense strand oligonucleotide
agtgacccaggaacaatattt

SEQ ID NO: 57 - DNA, 21 bp, artificial
  siRNA sense strand oligonucleotide
caatatgaattatactgtgtt

SEQ ID NO: 58 - DNA, 21 bp, artificial
  siRNA sense strand oligonucleotide
tatgaattatactgtgagatt

SEQ ID NO: 59 - DNA, 21 bp, artificial
  siRNA sense strand oligonucleotide
ttatactgtgagatgggcttt

SEQ ID NO: 60 - DNA, 21 bp, artificial
  siRNA sense strand oligonucleotide
tgataaggatgtaaagatttt

SEQ ID NO: 61 - DNA, 21 bp, artificial
  siRNA sense strand oligonucleotide
ggatgtaaagattgagccctt

SEQ ID NO: 62 - DNA, 21 bp, artificial
  siRNA sense strand oligonucleotide
agattgagccctgtggacatt

SEQ ID NO: 63 - DNA, 21 bp, artificial
  siRNA sense strand oligonucleotide
tcagaaggtcagggctgtctt

SEQ ID NO: 64 - DNA, 21 bp, artificial
  siRNA sense strand oligonucleotide
ggtcagggctgtcctttcttt

SEQ ID NO: 65 - DNA, 21 bp, artificial
  siRNA sense strand oligonucleotide
attaaaggtactgaacccatt

SEQ ID NO: 66 - DNA, 21 bp, artificial
  siRNA sense strand oligonucleotide
aggtactgaacccatcgtgtt

SEQ ID NO: 67 - DNA, 21 bp, artificial
  siRNA sense strand oligonucleotide
cccatcgtggtagatccgttt

SEQ ID NO: 68 - DNA, 21 bp, artificial
  siRNA sense strand oligonucleotide
attatgatgatgatgatgatt

SEQ ID NO: 69 - DNA, 21 bp, artificial
  siRNA sense strand oligonucleotide
cgagctgatgatactctcttt

SEQ ID NO: 70 - DNA, 21 bp, artificial
  siRNA sense strand oligonucleotide
ggaattggctggtgccaagtt

SEQ ID NO: 71 - DNA, 21 bp, artificial
  siRNA sense strand oligonucleotide
ttggctggtgccaaggtggtt

SEQ ID NO: 72 - DNA, 21 bp, artificial
  siRNA sense strand oligonucleotide
cggccgccttctccattcttt

SEQ ID NO: 73 - DNA, 21 bp, artificial
  siRNA sense strand oligonucleotide
gtgcttctgctcttggaactt

SEQ ID NO: 74 - DNA, 21 bp, artificial
  siRNA sense strand oligonucleotide
ctgcttctaaggctgcttctt

SEQ ID NO: 75 - DNA, 21 bp, artificial
  siRNA sense strand oligonucleotide
ggctgcttctggctccctttt

SEQ ID NO: 76 - DNA, 21 bp, artificial
  siRNA sense strand oligonucleotide
agacaaaccattgccagtatt

SEQ ID NO: 77 - DNA, 21 bp, artificial
  siRNA sense strand oligonucleotide
accattgccagtacctccctt

SEQ ID NO: 78 - DNA, 21 bp, artificial
  siRNA sense strand oligonucleotide
tcccgacctcaaagacgcctt

SEQ ID NO: 79 - DNA, 21 bp, artificial
  siRNA sense strand oligonucleotide
agacgccccttgccttgtatt

SEQ ID NO: 80 - DNA, 21 bp, artificial
  siRNA sense strand oligonucleotide
tccccaaagtaccagtatctt

SEQ ID NO: 81 - DNA, 21 bp, artificial
  siRNA sense strand oligonucleotide
agtaccagtatctgccccatt

SEQ ID NO: 82 - DNA, 21 bp, artificial
  siRNA sense strand oligonucleotide
gttccagtgatccctggactt

SEQ ID NO: 83 - DNA, 21 bp, artificial
  siRNA sense strand oligonucleotide
gagaattaaccaaccggcatt

SEQ ID NO: 84 - DNA, 21 bp, artificial
  siRNA sense strand oligonucleotide
ttaaccaaccggcactcactt

SEQ ID NO: 85 - DNA, 21 bp, artificial
  siRNA sense strand oligonucleotide
ccaaccggcactcacttcctt

SEQ ID NO: 86 - DNA, 21 bp, artificial
  siRNA sense strand oligonucleotide
ccggcactcacttccattttt

SEQ ID NO: 87 - DNA, 21 bp, artificial
  siRNA sense strand oligonucleotide
atggagcccagaccagatgtt

SEQ ID NO: 88 - DNA, 21 bp, artificial
  siRNA sense strand oligonucleotide
gcacgttcagtctggatactt

SEQ ID NO: 89 - DNA, 21 bp, artificial
  siRNA sense strand oligonucleotide
tagcagcccattagtaggttt

SEQ ID NO: 90 - DNA, 21 bp, artificial
  siRNA sense strand oligonucleotide
tcaaaccttcctcatctgctt

SEQ ID NO: 91 - DNA, 21 bp, artificial
  siRNA sense strand oligonucleotide
accttcctcatctgccaattt

SEQ ID NO: 92 - DNA, 21 bp, artificial
  siRNA sense strand oligonucleotide
tgccatttattctctggcttt

SEQ ID NO: 93 - DNA, 21 bp, artificial
  siRNA sense strand oligonucleotide
ctgccacctggggagcaattt

SEQ ID NO: 94 - DNA, 21 bp, artificial
  siRNA sense strand oligonucleotide
tgtgagggtgaagaggacatt

SEQ ID NO: 95 - DNA, 21 bp, artificial
  siRNA sense strand oligonucleotide
gaggacacagagtacatgatt

SEQ ID NO: 96 - DNA, 21 bp, artificial
  siRNA sense strand oligonucleotide
gcaatgtataatattcagttt

SEQ ID NO: 97 - DNA, 21 bp, artificial
  siRNA sense strand oligonucleotide
tgtataatattcagtcccatt

SEQ ID NO: 98 - DNA, 21 bp, artificial
  siRNA sense strand oligonucleotide
tattcagtcccaggcgccatt

SEQ ID NO: 99 - DNA, 21 bp, artificial
  siRNA sense strand oligonucleotide
cactggtcccgaggagtcatt

SEQ ID NO: 100 - DNA, 21 bp, artificial
  siRNA sense strand oligonucleotide
tgaggatgatgggtatgattt

SEQ ID NO: 101 - DNA, 21 bp, artificial
  siRNA sense strand oligonucleotide
ctctctcagatatctctaatt

SEQ ID NO: 102 - DNA, 21 bp, artificial
  siRNA sense strand oligonucleotide
tgccagctcctcctttggctt

SEQ ID NO: 103 - DNA, 21 bp, artificial
  siRNA sense strand oligonucleotide
caaatgtcactgaaggttctt

SEQ ID NO: 104 - DNA, 21 bp, artificial
  siRNA sense strand oligonucleotide
atgtcactgaaggttcccatt

SEQ ID NO: 105 - DNA, 21 bp, artificial
  siRNA sense strand oligonucleotide
ggttcccaagttcccgagatt

SEQ ID NO: 106 - DNA, 21 bp, artificial
  siRNA sense strand oligonucleotide
gttcccgagaggcctccaatt

SEQ ID NO: 107 - DNA, 21 bp, artificial
  siRNA sense strand oligonucleotide
ccattcccgcggagaatcatt

SEQ ID NO: 108 - DNA, 21 bp, artificial
  siRNA sense strand oligonucleotide
tcaactctgaacggaaagctt

SEQ ID NO: 109 - DNA, 21 bp, artificial
  siRNA sense strand oligonucleotide
ctctgaacggaaagctggctt

SEQ ID NO: 110 - DNA, 21 bp, artificial
  siRNA sense strand oligonucleotide
cggaaagctggcagctgtctt

SEQ ID NO: 111 - DNA, 21 bp, artificial
  siRNA sense strand oligonucleotide
agctggcagctgtcagcaatt

SEQ ID NO: 112 - DNA, 21 bp, artificial
  siRNA sense strand oligonucleotide
cctcatgagtcaggggtactt

SEQ ID NO: 113 - DNA, 21 bp, artificial
  siRNA sense strand oligonucleotide
agctttggtcattgcccagtt

SEQ ID NO: 114 - DNA, 21 bp, artificial
  siRNA sense strand oligonucleotide
caacatcgagatggccaaatt

SEQ ID NO: 115 - DNA, 21 bp, artificial
  siRNA sense strand oligonucleotide
acatcctccgggaatttgttt

SEQ ID NO: 116 - DNA, 21 bp, artificial
  siRNA sense strand oligonucleotide
tttgtttccatttcttctctt

SEQ ID NO: 117 - DNA, 21 bp, artificial
  siRNA sense strand oligonucleotide
gtggcacctagaagggcagtt

SEQ ID NO: 118 - DNA, 21 bp, artificial
  siRNA sense strand oligonucleotide
gggcaggagttcctttggttt

SEQ ID NO: 119 - DNA, 21 bp, artificial
  siRNA sense strand oligonucleotide
gtcttgccctctctgtgggtt

SEQ ID NO: 120 - DNA, 21 bp, artificial
  siRNA sense strand oligonucleotide
gatttcaaagtggtgaaattt

SEQ ID NO: 121 - DNA, 21 bp, artificial
  siRNA sense strand oligonucleotide
tggagcagctagtatgttttt

SEQ ID NO: 122 - DNA, 21 bp, artificial
  siRNA antisense strand oligonucleotide
cccagagctcttcttcacgtt

SEQ ID NO: 123 - DNA, 21 bp, artificial
  siRNA antisense strand oligonucleotide
gcacttctccaccatcttctt

SEQ ID NO: 124 - DNA, 21 bp, artificial
  siRNA antisense strand oligonucleotide
ccagcacttctccaccatctt

SEQ ID NO: 125 - DNA, 21 bp, artificial
  siRNA antisense strand oligonucleotide
gtccatgagcttccagcactt

SEQ ID NO: 126 - DNA, 21 bp, artificial
  siRNA antisense strand oligonucleotide
caccaccttgtccatgagctt

SEQ ID NO: 127 - DNA, 21 bp, artificial
  siRNA antisense strand oligonucleotide
ctgacacaaccgcaccacctt

SEQ ID NO: 128 - DNA, 21 bp, artificial
  siRNA antisense strand oligonucleotide
ctttagcgccagctttgggtt

SEQ ID NO: 129 - DNA, 21 bp, artificial
  siRNA antisense strand oligonucleotide
ctttagcgccagctttgggtt

SEQ ID NO: 130 - DNA, 21 bp, artificial
  siRNA antisense strand oligonucleotide
ctattctttagcgccagcttt

SEQ ID NO: 131 - DNA, 21 bp, artificial
  siRNA antisense strand oligonucleotide
atataaggtgggctattcttt

SEQ ID NO: 132 - DNA, 21 bp, artificial
  siRNA antisense strand oligonucleotide
taagatataaggtgggctatt

SEQ ID NO: 133 - DNA, 21 bp, artificial
  siRNA antisense strand oligonucleotide
tccatcttcccctcatatctt

SEQ ID NO: 134 - DNA, 21 bp, artificial
  siRNA antisense strand oligonucleotide
ttctccaagtgtctccatctt

SEQ ID NO: 135 - DNA, 21 bp, artificial
  siRNA antisense strand oligonucleotide
aggcttatggtttgcttagtt

SEQ ID NO: 136 - DNA, 21 bp, artificial
  siRNA antisense strand oligonucleotide
gaagaggcttatggtttgctt

SEQ ID NO: 137 - DNA, 21 bp, artificial
  siRNA antisense strand oligonucleotide
ccttgaagaggcttatggttt

SEQ ID NO: 138 - DNA, 21 bp, artificial
  siRNA antisense strand oligonucleotide
tttccctccttgaagaggctt

SEQ ID NO: 139 - DNA, 21 bp, artificial
  siRNA antisense strand oligonucleotide
tctcctcatacattctttctt

SEQ ID NO: 140 - DNA, 21 bp, artificial
  siRNA antisense strand oligonucleotide
aattctcctcatacattcttt

SEQ ID NO: 141 - DNA, 21 bp, artificial
  siRNA antisense strand oligonucleotide
tgagaattctcctcatacatt

SEQ ID NO: 142 - DNA, 21 bp, artificial
  siRNA antisense strand oligonucleotide
gtttcgcctaggctgagaatt

SEQ ID NO: 143 - DNA, 21 bp, artificial
  siRNA antisense strand oligonucleotide
agggacagtttggttaggttt

SEQ ID NO: 144 - DNA, 21 bp, artificial
  siRNA antisense strand oligonucleotide
aagatgagggacagtttggtt

SEQ ID NO: 145 - DNA, 21 bp, artificial
  siRNA antisense strand oligonucleotide
gctgaagatgagggacagttt

SEQ ID NO: 146 - DNA, 21 bp, artificial
  siRNA antisense strand oligonucleotide
gtccacttggaaagattcctt

SEQ ID NO: 147 - DNA, 21 bp, artificial
  siRNA antisense strand oligonucleotide
aagagtccacttggaaagatt

SEQ ID NO: 148 - DNA, 21 bp, artificial
  siRNA antisense strand oligonucleotide
tctccctgaaagagtccactt

SEQ ID NO: 149 - DNA, 21 bp, artificial
  siRNA antisense strand oligonucleotide
aaattccgcagcatctgcttt

SEQ ID NO: 150 - DNA, 21 bp, artificial
  siRNA antisense strand oligonucleotide
cttccaagggactattgtctt

SEQ ID NO: 151 - DNA, 21 bp, artificial
  siRNA antisense strand oligonucleotide
aagctcttccaagggactatt

SEQ ID NO: 152 - DNA, 21 bp, artificial
  siRNA antisense strand oligonucleotide
tagagcctgtcgaaagctctt

SEQ ID NO: 153 - DNA, 21 bp, artificial
  siRNA antisense strand oligonucleotide
cagaactgatgggatgcactt

SEQ ID NO: 154 - DNA, 21 bp, artificial
  siRNA antisense strand oligonucleotide
ggtcagatcaatagtggattt

SEQ ID NO: 155 - DNA, 21 bp, artificial
  siRNA antisense strand oligonucleotide
gtcgggtaaagatgtcaaatt

SEQ ID NO: 156 - DNA, 21 bp, artificial
  siRNA antisense strand oligonucleotide
tacagcaaggctgttccaatt

SEQ ID NO: 157 - DNA, 21 bp, artificial
  siRNA antisense strand oligonucleotide
atgagttacagcaaggctgtt

SEQ ID NO: 158 - DNA, 21 bp, artificial
  siRNA antisense strand oligonucleotide
gccatgtagccaggatgagtt

SEQ ID NO: 159 - DNA, 21 bp, artificial
  siRNA antisense strand oligonucleotide
tctggagccgagctttcactt

SEQ ID NO: 160 - DNA, 21 bp, artificial
  siRNA antisense strand oligonucleotide
gaatttctggagccgagcttt

SEQ ID NO: 161 - DNA, 21 bp, artificial
  siRNA antisense strand oligonucleotide
gccaggtttgtgaatgaattt

SEQ ID NO: 162 - DNA, 21 bp, artificial
  siRNA antisense strand oligonucleotide
gaagatataactgccaggttt

SEQ ID NO: 163 - DNA, 21 bp, artificial
  siRNA antisense strand oligonucleotide
agggattgtctggagaatgtt

SEQ ID NO: 164 - DNA, 21 bp, artificial
  siRNA antisense strand oligonucleotide
agaggtttattgtgagggatt

SEQ ID NO: 165 - DNA, 21 bp, artificial
  siRNA antisense strand oligonucleotide
tgcttggaagagaggtttatt

SEQ ID NO: 166 - DNA, 21 bp, artificial
  siRNA antisense strand oligonucleotide
cagtgcttggaagagaggttt

SEQ ID NO: 167 - DNA, 21 bp, artificial
  siRNA antisense strand oligonucleotide
tgaagccatcaatcagtgctt

SEQ ID NO: 168 - DNA, 21 bp, artificial
  siRNA antisense strand oligonucleotide
caggaaacaaatagaagcctt

SEQ ID NO: 169 - DNA, 21 bp, artificial
  siRNA antisense strand oligonucleotide
gtcagatcaggattctgattt

SEQ ID NO: 170 - DNA, 21 bp, artificial
  siRNA antisense strand oligonucleotide
taagccagtcagatcaggatt

SEQ ID NO: 171 - DNA, 21 bp, artificial
  siRNA antisense strand oligonucleotide
tatggtcttggggagttggtt

SEQ ID NO: 172 - DNA, 21 bp, artificial
  siRNA antisense strand oligonucleotide
ttgatatggtcttggggagtt

SEQ ID NO: 173 - DNA, 21 bp, artificial
  siRNA antisense strand oligonucleotide
gggtcactttgatatggtctt

SEQ ID NO: 174 - DNA, 21 bp, artificial
  siRNA antisense strand oligonucleotide
atattgttcctgggtcacttt

SEQ ID NO: 175 - DNA, 21 bp, artificial
  siRNA antisense strand oligonucleotide
cacagtataattcatattgtt

SEQ ID NO: 176 - DNA, 21 bp, artificial
  siRNA antisense strand oligonucleotide
tctcacagtataattcatatt

SEQ ID NO: 177 - DNA, 21 bp, artificial
  siRNA antisense strand oligonucleotide
agcccatctcacagtataatt

SEQ ID NO: 178 - DNA, 21 bp, artificial
  siRNA antisense strand oligonucleotide
aatctttacatccttatcatt

SEQ ID NO: 179 - DNA, 21 bp, artificial
  siRNA antisense strand oligonucleotide
gggctcaatctttacatcctt

SEQ ID NO: 180 - DNA, 21 bp, artificial
  siRNA antisense strand oligonucleotide
tgtccacagggctcaatcttt

SEQ ID NO: 181 - DNA, 21 bp, artificial
  siRNA antisense strand oligonucleotide
gacagccctgaccttctgatt

SEQ ID NO: 182 - DNA, 21 bp, artificial
  siRNA antisense strand oligonucleotide
agaaaggacagccctgacctt

SEQ ID NO: 183 - DNA, 21 bp, artificial
  siRNA antisense strand oligonucleotide
tgggttcagtacctttaattt

SEQ ID NO: 184 - DNA, 21 bp, artificial
  siRNA antisense strand oligonucleotide
cacgatgggttcagtaccttt

SEQ ID NO: 185 - DNA, 21 bp, artificial
  siRNA antisense strand oligonucleotide
acggatctaccacgatgggtt

SEQ ID NO: 186 - DNA, 21 bp, artificial
  siRNA antisense strand oligonucleotide
tcatcatcatcatcataattt

SEQ ID NO: 187 - DNA, 21 bp, artificial
  siRNA antisense strand oligonucleotide
agagagtatcatcagctcgtt

SEQ ID NO: 188 - DNA, 21 bp, artificial
  siRNA antisense strand oligonucleotide
cttggcaccagccaattcctt

SEQ ID NO: 189 - DNA, 21 bp, artificial
  siRNA antisense strand oligonucleotide
ccaccttggcaccagccaatt

SEQ ID NO: 190 - DNA, 21 bp, artificial
  siRNA antisense strand oligonucleotide
agaatggagaaggcggccgtt

SEQ ID NO: 191 - DNA, 21 bp, artificial
  siRNA antisense strand oligonucleotide
gttccaagagcagaagcactt

SEQ ID NO: 192 - DNA, 21 bp, artificial
  siRNA antisense strand oligonucleotide
gaagcagccttagaagcagtt

SEQ ID NO: 193 - DNA, 21 bp, artificial
  siRNA antisense strand oligonucleotide
aagggagccagaagcagcctt

SEQ ID NO: 194 - DNA, 21 bp, artificial
  siRNA antisense strand oligonucleotide
tactggcaatggtttgtcttt

SEQ ID NO: 195 - DNA, 21 bp, artificial
  siRNA antisense strand oligonucleotide
gggaggtactggcaatggttt

SEQ ID NO: 196 - DNA, 21 bp, artificial
  siRNA antisense strand oligonucleotide
ggcgtctttgaggtcgggatt

SEQ ID NO: 197 - DNA, 21 bp, artificial
  siRNA antisense strand oligonucleotide
tacaaggcaaggggcgtcttt

SEQ ID NO: 198 - DNA, 21 bp, artificial
  siRNA antisense strand oligonucleotide
gatactggtactttggggatt

SEQ ID NO: 199 - DNA, 21 bp, artificial
  siRNA antisense strand oligonucleotide
tggggcagatactggtacttt

SEQ ID NO: 200 - DNA, 21 bp, artificial
  siRNA antisense strand oligonucleotide
gtccagggatcactggaactt

SEQ ID NO: 201 - DNA, 21 bp, artificial
  siRNA antisense strand oligonucleotide
tgccggttggttaattctctt

SEQ ID NO: 202 - DNA, 21 bp, artificial
  siRNA antisense strand oligonucleotide
gtgagtgccggttggttaatt

SEQ ID NO: 203 - DNA, 21 bp, artificial
  siRNA antisense strand oligonucleotide
ggaagtgagtgccggttggtt

SEQ ID NO: 204 - DNA, 21 bp, artificial
  siRNA antisense strand oligonucleotide
aaatggaagtgagtgccggtt

SEQ ID NO: 205 - DNA, 21 bp, artificial
  siRNA antisense strand oligonucleotide
catctggtctgggctccattt

SEQ ID NO: 206 - DNA, 21 bp, artificial
  siRNA antisense strand oligonucleotide
gtatccagactgaacgtgctt

SEQ ID NO: 207 - DNA, 21 bp, artificial
  siRNA antisense strand oligonucleotide
acctactaatgggctgctatt

SEQ ID NO: 208 - DNA, 21 bp, artificial
  siRNA antisense strand oligonucleotide
gcagatgaggaaggtttgatt

SEQ ID NO: 209 - DNA, 21 bp, artificial
  siRNA antisense strand oligonucleotide
attggcagatgaggaaggttt

SEQ ID NO: 210 - DNA, 21 bp, artificial
  siRNA antisense strand oligonucleotide
agccagagaataaatggcatt

SEQ ID NO: 211 - DNA, 21 bp, artificial
  siRNA antisense strand oligonucleotide
attgctccccaggtggcagtt

SEQ ID NO: 212 - DNA, 21 bp, artificial
  siRNA antisense strand oligonucleotide
tgtcctcttcaccctcacatt

SEQ ID NO: 213 - DNA, 21 bp, artificial
  siRNA antisense strand oligonucleotide
tcatgtactctgtgtcctctt

SEQ ID NO: 214 - DNA, 21 bp, artificial
  siRNA antisense strand oligonucleotide
actgaatattatacattgctt

SEQ ID NO: 215 - DNA, 21 bp, artificial
  siRNA antisense strand oligonucleotide
tgggactgaatattatacatt

SEQ ID NO: 216 - DNA, 21 bp, artificial
  siRNA antisense strand oligonucleotide
tggcgcctgggactgaatatt

SEQ ID NO: 217 - DNA, 21 bp, artificial
  siRNA antisense strand oligonucleotide
tgactcctcgggaccagtgtt

SEQ ID NO: 218 - DNA, 21 bp, artificial
  siRNA antisense strand oligonucleotide
atcatacccatcatcctcatt

SEQ ID NO: 219 - DNA, 21 bp, artificial
  siRNA antisense strand oligonucleotide
ttagagatatctgagagagtt

SEQ ID NO: 220 - DNA, 21 bp, artificial
  siRNA antisense strand oligonucleotide
gccaaaggaggagctggcatt

SEQ ID NO: 221 - DNA, 21 bp, artificial
  siRNA antisense strand oligonucleotide
gaaccttcagtgacatttgtt

SEQ ID NO: 222 - DNA, 21 bp, artificial
  siRNA antisense strand oligonucleotide
tgggaaccttcagtgacattt

SEQ ID NO: 223 - DNA, 21 bp, artificial
  siRNA antisense strand oligonucleotide
tctcgggaacttgggaacctt

SEQ ID NO: 224 - DNA, 21 bp, artificial
  siRNA antisense strand oligonucleotide
ttggaggcctctcgggaactt

SEQ ID NO: 225 - DNA, 21 bp, artificial
  siRNA antisense strand oligonucleotide
tgattctccgcgggaatggtt

SEQ ID NO: 226 - DNA, 21 bp, artificial
  siRNA antisense strand oligonucleotide
gctttccgttcagagttgatt

SEQ ID NO: 227 - DNA, 21 bp, artificial
  siRNA antisense strand oligonucleotide
gccagctttccgttcagagtt

SEQ ID NO: 228 - DNA, 21 bp, artificial
  siRNA antisense strand oligonucleotide
gacagctgccagctttccgtt

SEQ ID NO: 229 - DNA, 21 bp, artificial
  siRNA antisense strand oligonucleotide
ttgctgacagctgccagcttt

SEQ ID NO: 230 - DNA, 21 bp, artificial
  siRNA antisense strand oligonucleotide
gtacccctgactcatgaggtt

SEQ ID NO: 231 - DNA, 21 bp, artificial
  siRNA antisense strand oligonucleotide
ctgggcaatgaccaaagcttt

SEQ ID NO: 232 - DNA, 21 bp, artificial
  siRNA antisense strand oligonucleotide
tttggccatctcgatgttgtt

SEQ ID NO: 233 - DNA, 21 bp, artificial
  siRNA antisense strand oligonucleotide
acaaattcccggaggatgttt

SEQ ID NO: 234 - DNA, 21 bp, artificial
  siRNA antisense strand oligonucleotide
gagaagaaatggaaacaaatt

SEQ ID NO: 235 - DNA, 21 bp, artificial
  siRNA antisense strand oligonucleotide
ctgcccttctaggtgccactt

SEQ ID NO: 236 - DNA, 21 bp, artificial
  siRNA antisense strand oligonucleotide
accaaaggaactcctgccctt

SEQ ID NO: 237 - DNA, 21 bp, artificial
  siRNA antisense strand oligonucleotide
cccacagagagggcaagactt

SEQ ID NO: 238 - DNA, 21 bp, artificial
  siRNA antisense strand oligonucleotide
atttcaccactttgaaatctt

SEQ ID NO: 239 - DNA, 21 bp, artificial
  siRNA antisense strand oligonucleotide
aaacatactagctgctccatt

SEQ ID NO: 240 - DNA, 3 bp, artificial
  shRNA loop sequence
ccc

SEQ ID NO: 241 - DNA, 4 bp, artificial
  shRNA loop sequence
ttcg

SEQ ID NO: 242 - DNA, 5 bp, artificial
  shRNA loop sequence
ccacc

SEQ ID NO: 243 - DNA, 6 bp, artificial
  shRNA loop sequence
ctcgag

SEQ ID NO: 244 - DNA, 6 bp, artificial
  shRNA loop sequence
aagctt

SEQ ID NO: 245 - DNA, 7 bp, artificial
  shRNA loop sequence
ccacacc

SEQ ID NO: 246 - DNA, 9 bp, artificial
  shRNA loop sequence
ttcaagaga

SEQ ID NO: 247 - DNA, 2721 bp, artificial
  c-Cbl G306E mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgagtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 248 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIEYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 249 - DNA, 2721 bp, artificial
  c-Cbl C381A mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactagctaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 250 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLAKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 251 - DNA, 2721 bp, artificial
  c-Cbl Y700F mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagttcatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 252 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEFMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 253 - DNA, 2721 bp, artificial
  c-Cbl Y731F mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtttgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 254 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTFEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 255 - DNA, 2721 bp, artificial
  c-Cbl Y774F mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtttgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 256 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGFDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 257 - DNA, 2721 bp, artificial
  c-Cbl Y700F/Y731F/Y774F triple mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagttcatgactccctcttccaggcctctacggcctttggatacatcccagagttcacgagcatgtgattgcgaccagcagattgatagctgtacgtttgaagcaatgtataatattcagtcccaggcgccatctatcaccgagagcagcacctttggtgaagggaatttggccgcagcccatgccaacactggtcccgaggagtcagaaaatgaggatgatgggtttgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag

SEQ ID NO: 258 - protein, 906 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEFMTPSSRPLRPLDTSQSSRACDCDQQIDSCTFEAMYNIQSQAPSITESSTFGEGNLAAAHANTGPEESENEDDGFDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 259 - DNA, 1443 bp, artificial
  c-Cbl480  mutant
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggtag

SEQ ID NO: 260 - protein, 480 aa, artificial
  Synthetic Construct
MAGNVKKSSGAGGGTGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVER

SEQ ID NO: 261 - DNA, 2721 bp, Homo sapiens
atggccggcaacgtgaagaagagctctggggccgggggcggcacgggctccgggggctcgggttcgggtggcctgattgggctcatgaaggacgccttccagccgcaccaccaccaccaccaccacctcagcccccacccgccggggacggtggacaagaagatggtggagaagtgctggaagctcatggacaaggtggtgcggttgtgtcagaacccaaagctggcgctaaagaatagcccaccttatatcttagacctgctaccagatacctaccagcatctccgtactatcttgtcaagatatgaggggaagatggagacacttggagaaaatgagtattttagggtgtttatggagaatttgatgaagaaaactaagcaaaccataagcctcttcaaggagggaaaagaaagaatgtatgaggagaattctcagcctaggcgaaacctaaccaaactgtccctcatcttcagccacatgctggcagaactaaaaggaatctttccaagtggactctttcagggagacacatttcggattactaaagcagatgctgcggaattttggagaaaagcttttggggaaaagacaatagtcccttggaagagctttcgacaggctctacatgaagtgcatcccatcagttctgggctggaggccatggctctgaaatccactattgatctgacctgcaatgattatatttcggtttttgaatttgacatctttacccgactctttcagccctggtcctctttgctcaggaattggaacagccttgctgtaactcatcctggctacatggcttttttgacgtatgacgaagtgaaagctcggctccagaaattcattcacaaacctggcagttatatcttccggctgagctgtactcgtctgggtcagtgggctattgggtatgttactgctgatgggaacattctccagacaatccctcacaataaacctctcttccaagcactgattgatggcttcagggaaggcttctatttgtttcctgatggacgaaatcagaatcctgatctgactggcttatgtgaaccaactccccaagaccatatcaaagtgacccaggaacaatatgaattatactgtgagatgggctccacattccaactatgtaaaatatgtgctgaaaatgataaggatgtaaagattgagccctgtggacacctcatgtgcacatcctgtcttacatcctggcaggaatcagaaggtcagggctgtcctttctgccgatgtgaaattaaaggtactgaacccatcgtggtagatccgtttgatcctagagggagtggcagcctgttgaggcaaggagcagagggagctccctccccaaattatgatgatgatgatgatgaacgagctgatgatactctcttcatgatgaaggaattggctggtgccaaggtggaacggccgccttctccattctccatggccccacaagcttcccttcccccggtgccaccacgacttgaccttctgccgcagcgagtatgtgttccctcaagtgcttctgctcttggaactgcttctaaggctgcttctggctcccttcataaagacaaaccattgccagtacctcccacacttcgagatcttccaccaccaccgcctccagaccggccatattctgttggagcagaatcccgacctcaaagacgccccttgccttgtacaccaggcgactgtccctccagagacaaactgccccctgtcccctctagccgccttggagactcatggctgccccggccaatccccaaagtaccagtatctgccccaagttccagtgatccctggacaggaagagaattaaccaaccggcactcacttccattttcattgccctcacaaatggagcccagaccagatgtgcctaggctcggaagcacgttcagtctggatacctccatgagtatgaatagcagcccattagtaggtccagagtgtgaccaccccaaaatcaaaccttcctcatctgccaatgccatttattctctggctgccagacctcttcctgtgccaaaactgccacctggggagcaatgtgagggtgaagaggacacagagtacatgactccctcttccaggcctctacggcctttggatacatcccagagttcacgagcatgtgattgcgaccagcagattgatagctgtacgtatgaagcaatgtataatattcagtcccaggcgccatctatcaccgagagcagcacctttggtgaagggaatttggccgcagcccatgccaacactggtcccgaggagtcagaaaatgaggatgatgggtatgatgtcccaaagccacctgtgccggccgtgctggcccgccgaactctctcagatatctctaatgccagctcctcctttggctggttgtctctggatggtgatcctacaacaaatgtcactgaaggttcccaagttcccgagaggcctccaaaaccattcccgcggagaatcaactctgaacggaaagctggcagctgtcagcaaggtagtggtcctgccgcctctgctgccaccgcctcacctcagctctccagtgagatcgagaacctcatgagtcaggggtactcctaccaggacatccagaaagctttggtcattgcccagaacaacatcgagatggccaaaaacatcctccgggaatttgtttccatttcttctcctgcccatgtagctacctag